(19) United States
(12) Patent Application Publication
(10) Pub. No.: US 2024/0067672 A1
(43) Pub. Date: Feb. 29, 2024

(54) FLAVONE DERIVATIVE FOR TREATING TUMORS AND USE THEREOF

(71) Applicant: SICHUAN FUSHENGYUAN TECHNOLOGY CO., LTD, Chengdu (CN)

(72) Inventors: Yongmei Xie, Chengdu (CN); Xiangrong Song, Chengdu (CN); Bo Luo, Chengdu (CN)

(21) Appl. No.: 17/542,693

(22) PCT Filed: Jun. 8, 2020
(86) PCT No.: PCT/CN2020/094830

§ 371 Date: Nov. 2, 2023

(30) Foreign Application Priority Data
Jun. 10, 2019 (CN) 201910494758.8

(51) Int. Cl. C07H 19/056; C07H 1/00; C07H 15/26

(52) U.S. Cl. CPC C07H 19/056; C07H 1/00; C07H 15/26

(57) ABSTRACT

Provided are a flavone derivative represented by formula I and a pharmaceutically acceptable salt, hydrate or solvate thereof. (I) In formula I, R₁ is selected from the group consisting of H, C₁₋₄ alkyl, amino and C₁₋₄ acyl; R₂ is isopentenyl or 2-hydroxy-isopentyl; R₃ is selected from the group consisting of H, methyl and deuterated methyl; R₄ is selected from the group consisting of C₁₋₄ alkyl, amino, C₁₋₄ acyl, and (II), wherein R₅ represents a monosaccharide residue or an oligosaccharide residue; L is selected from the group consisting of a polypeptide, C₁-C₂₀ linear alkyl or a derivative thereof, a derivative of C₁-C₂₀ linear or branched acyl, C₁-C₂₀ glycol or a derivative thereof, and (III), wherein Y is (IV), a is an integer from 0-100, b is an integer from 1-100, c is an integer from 1-10, d is an integer from 0-100, and e is an integer from 0-100. The flavone derivative according to the present invention exhibits an potent and broad-spectrum anti-cancer activity.

### TECHNICAL FIELD

The present disclosure relates to a novel flavone derivative and a pharmacologically acceptable salt, hydrate or solvate thereof. The derivative could he used as an antitumor drug.

### BACKGROUND ART

Tumor is a common disease threatening human life. Millions of people die from tumors every year in China and the number is increasing progressively. Tumor has become the first cause of death among urban populations. Therefore, it has been always the pursuit of the medical community to find safe and efficient antitumor drugs. Traditional chemotherapeutic drugs have obvious therapeutic effects on clinic, but they have the disadvantages of low specificity, low selectivity, causing obvious toxic side effects, and being prone to produce serious multidrug resistance of tumor, thus limiting their clinical applications.

Flavonoids, being a kind of natural phenolic compounds found in plants, have diverse structures and physicochemical properties and spread widely in plant kingdoms. Currently, it has been found that many natural flavonoids show certain antitumor activities, which, however, cannot meet the clinical demands. Because flavonoids has good safety in its structure, it has a great clinical value in improving the antitumor activity.

Epimedium flavone is the major effective component in the traditional Chinese medicine Epirnedium, which is the basic structural unit of f_cariin, also known as anhydroicaritin or Icaritin. It is a special flavone having an isopentenyl substituent at position 8 of the flavone ring, of which the structural formula is as below:

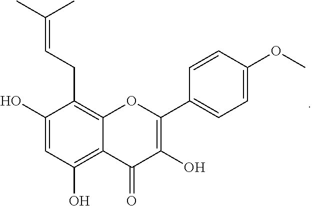

Chinese patent application No. 200780039276.9 discloses use of Icaritin in the preparation of drugs for abnormal cell proliferation, especially cancer drugs. Chinese patent application No. 03129242.9 proposes use of Icaritin in the preparation of selective estrogen receptor modulators. However, Icaritin, such an active medicine constituent has the disadvantages of poor solubility in water, very low bioavailability of oral administration, and the like. To solve the above problems, the present disclosure makes modifications on the structure of anhydroicaritin, obtaining a new generation of high efficient and low toxic flavonoid antitumor drugs.

### SUMMARY

To solve the above deficiencies and find better antitumor drugs, the present disclosure makes modifications on the structure of anhydroicaritin, finding that the flavone derivative represented by the structural formula I has better antitumor effects than icaritin

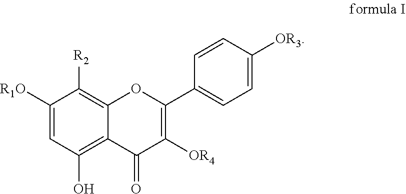

In formula I,

R₁ is selected from the group consisting of H, C₁₋₄ alkyl, amino, and C₁₋₄ acyl;

R₂ is isopentenyl or 2-hydroxy isopentyl;

R₃ is selected from the group consisting of H, methyl, and deuterated methyl; and

R₄ is selected from the group consisting of C₁₋₄ alkyl, amino, C₁₋₄ acyl, and

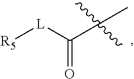

wherein R₅ represents a monosaccharide residue or an oligosaccharide residue; L is selected from the group consisting of a polypeptide, C₁-C₂₀ linear alkyl or a derivative thereof, a derivative of C₁-C₂₀ linear or branched acyl, C₁-C₂₀ glycol or a derivative thereof,

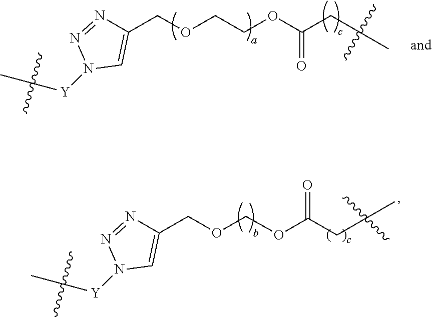

wherein Y is

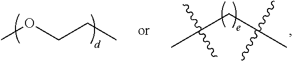

a is an integer from 0-100, b is an integer from 1-100, c is an integer from 1-10, d is an integer from 0-100, and e is an integer from 0-100.

In some embodiments, R₁ is H; R₂ is isopentenyl; R₃ is methyl; R₄ is

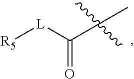

wherein R₅ is selected from the group consisting of the monosaccharide residue and the oligosaccharide residue, preferably the following monosaccharide residues 1-24:

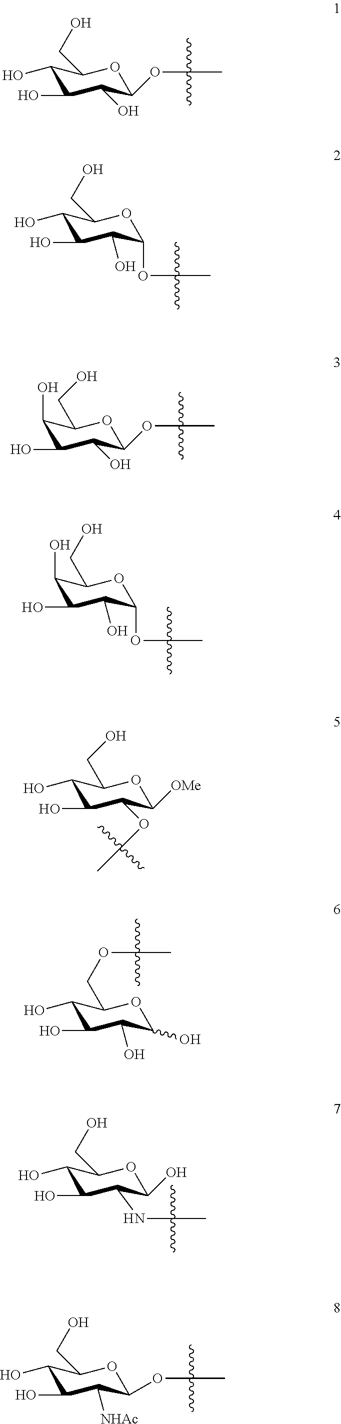

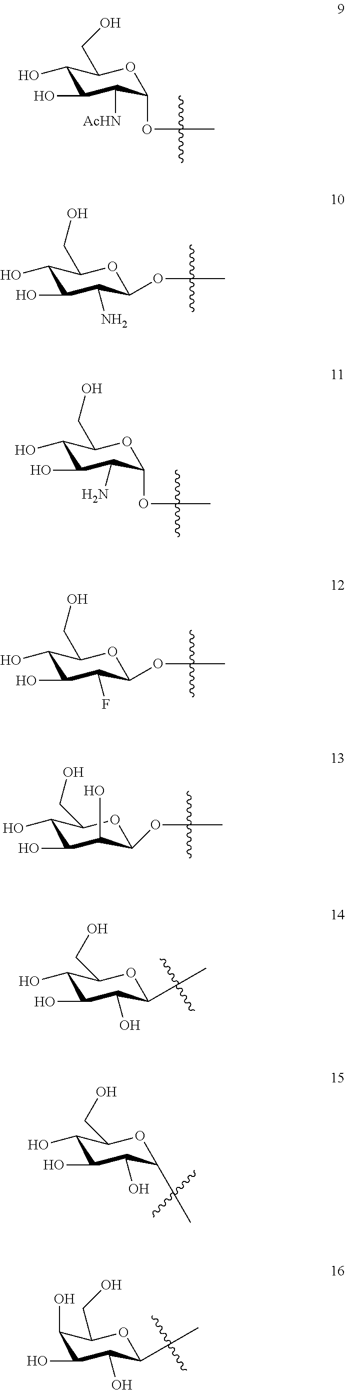

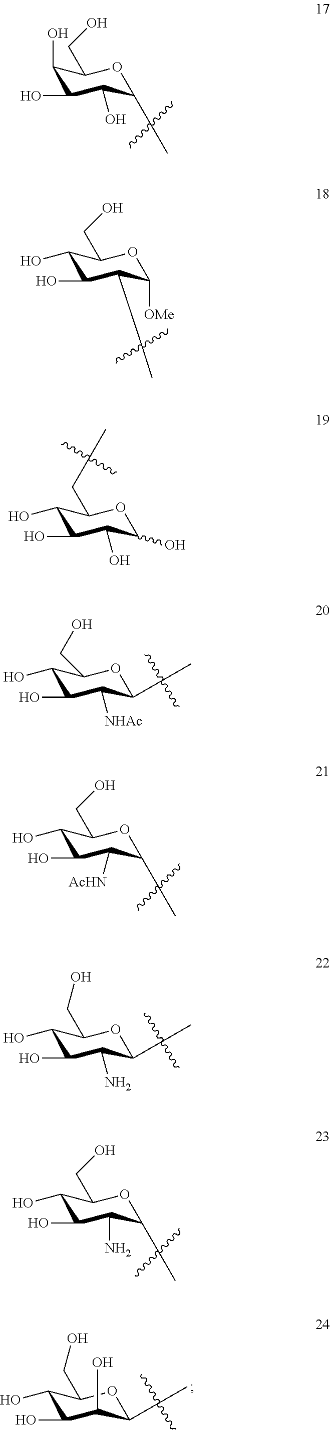

L is

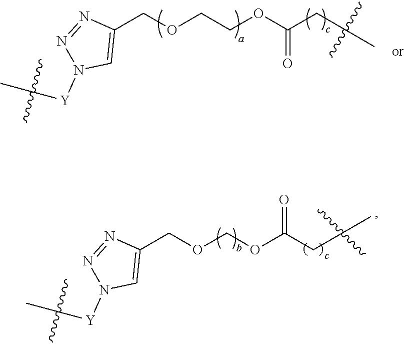

wherein Y is

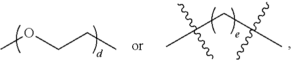

a is an integer from 0-20, b is an integer from 1-20, c is an integer from 1-10, d is an integer from 0-20, and e is an integer from 0-20.

In some embodiments, in formula I,

R₁ represents H;

R₂ represents isopentenyl;

R₃ represents methyl; and

R₄ represents

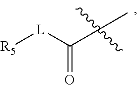

wherein R₅ is selected from the group consisting of monosaccharide residues 1, 2, 14 and 15; L is

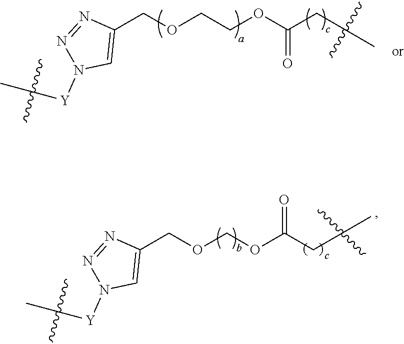

wherein Y is

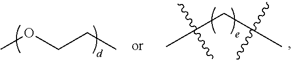

a is an integer from 0-20, b is an integer fro 1-20, c is an integer front 2-6, d is an integer from 0-20 and e is an integer from 0-20.

In some embodiments, the flavone derivative is one of the following compounds:

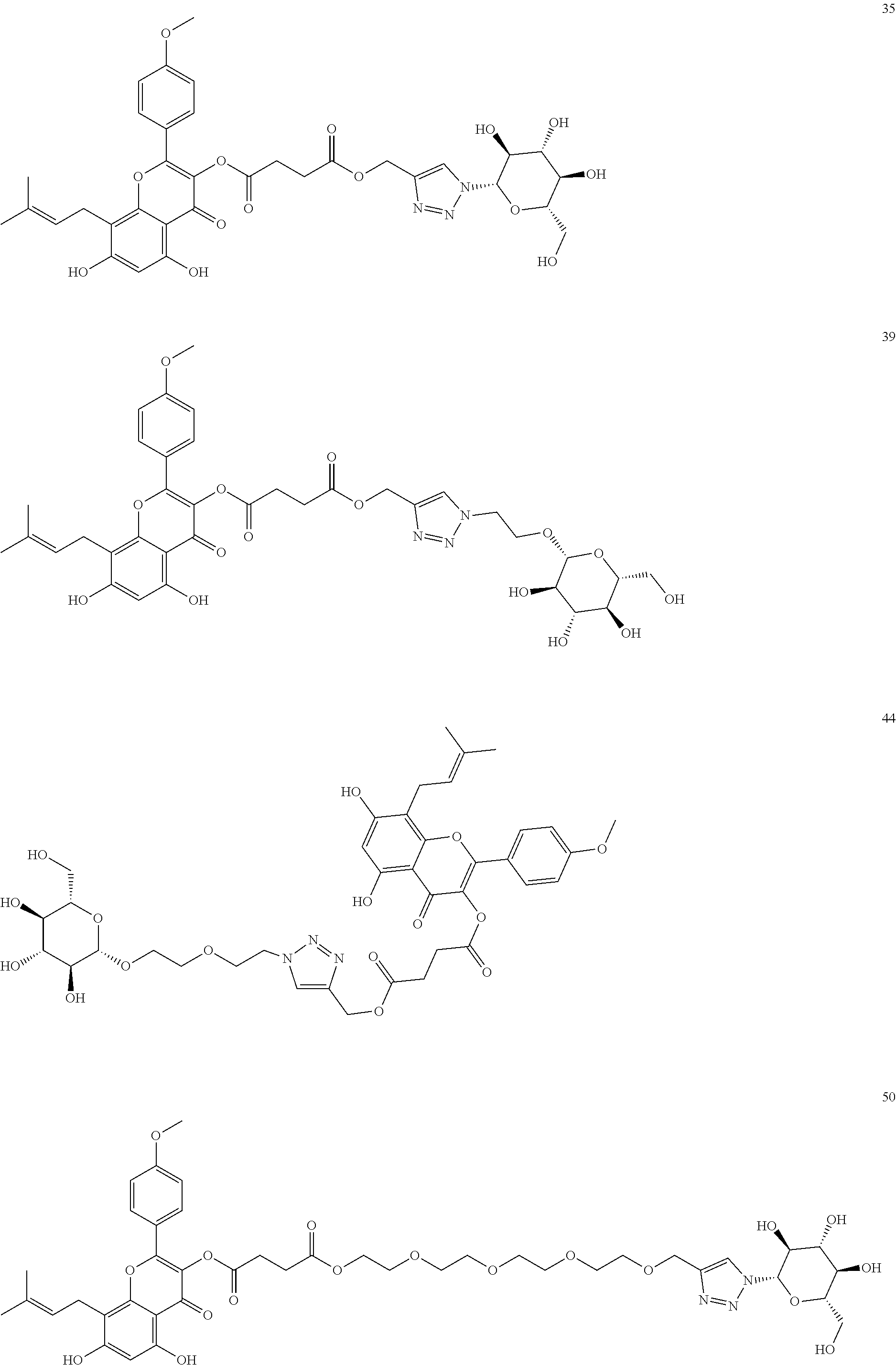

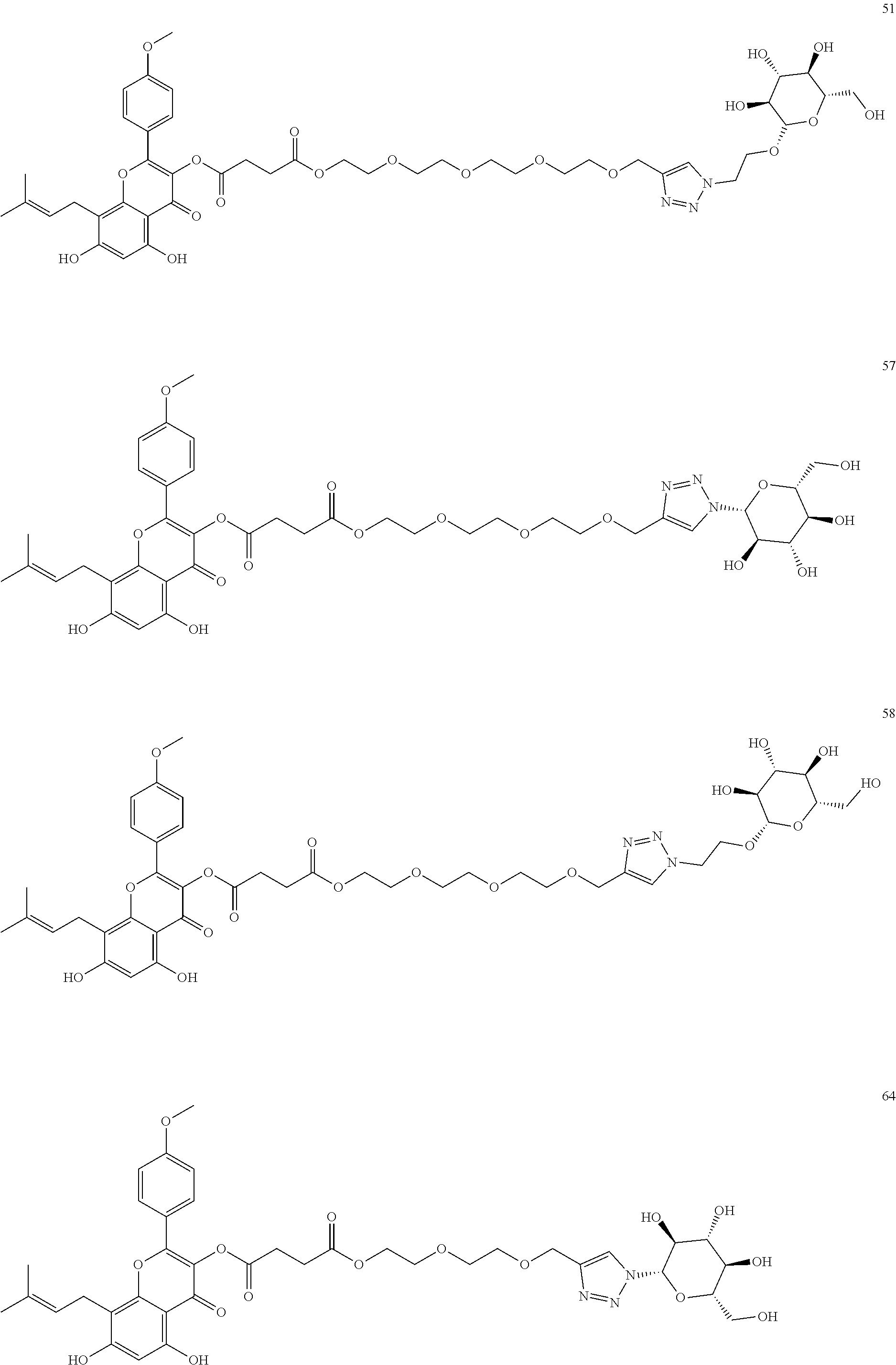

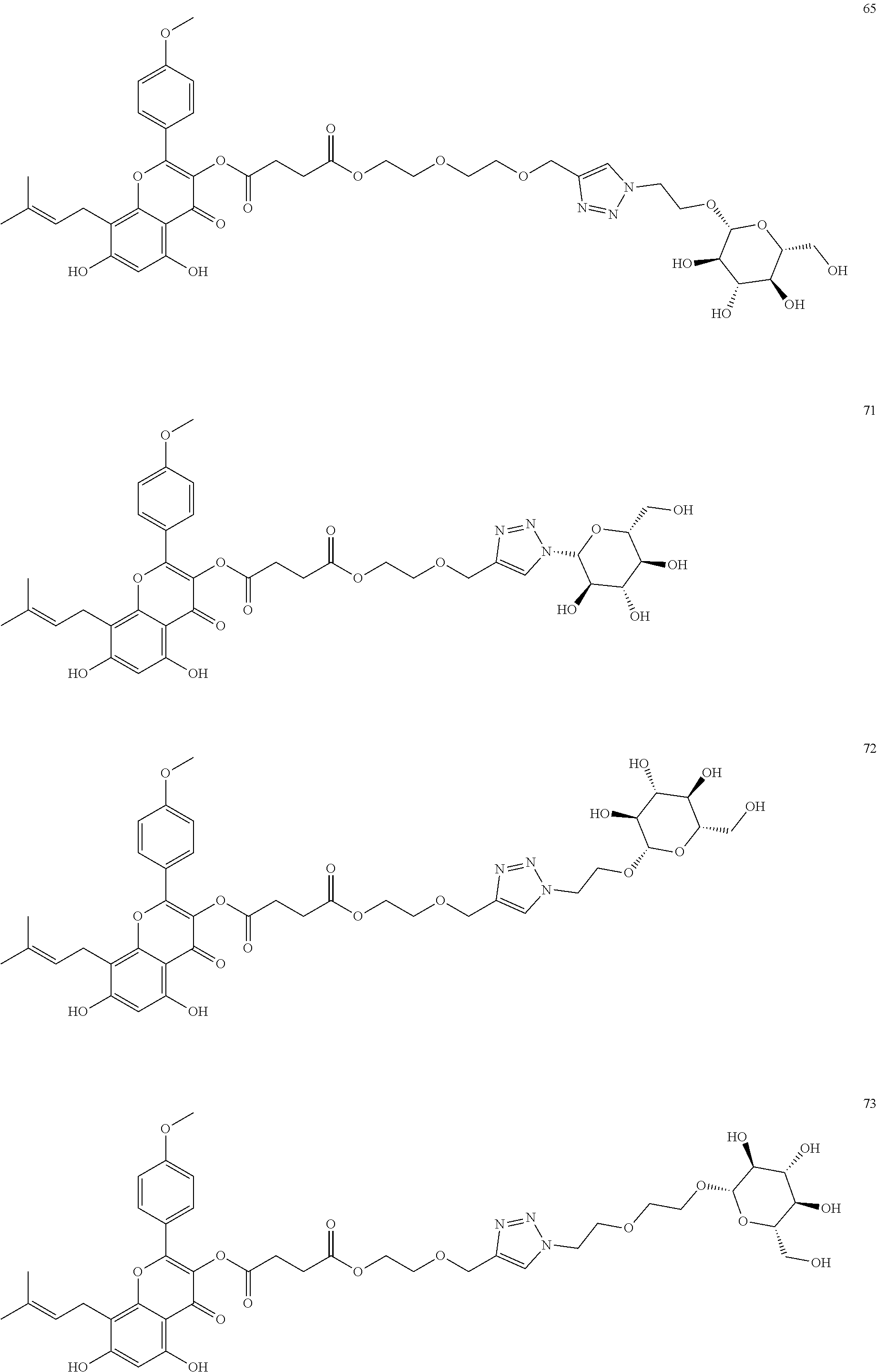

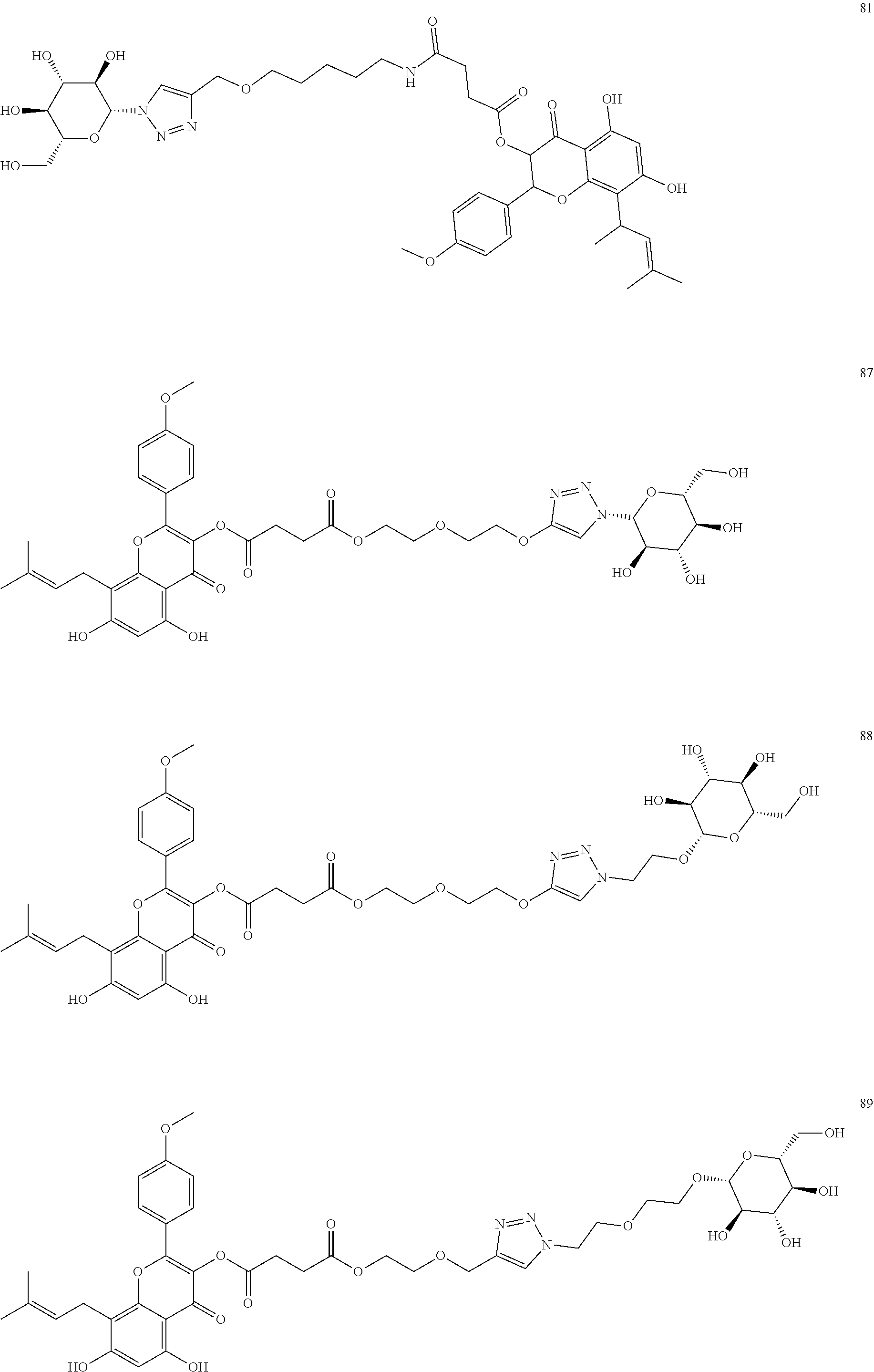

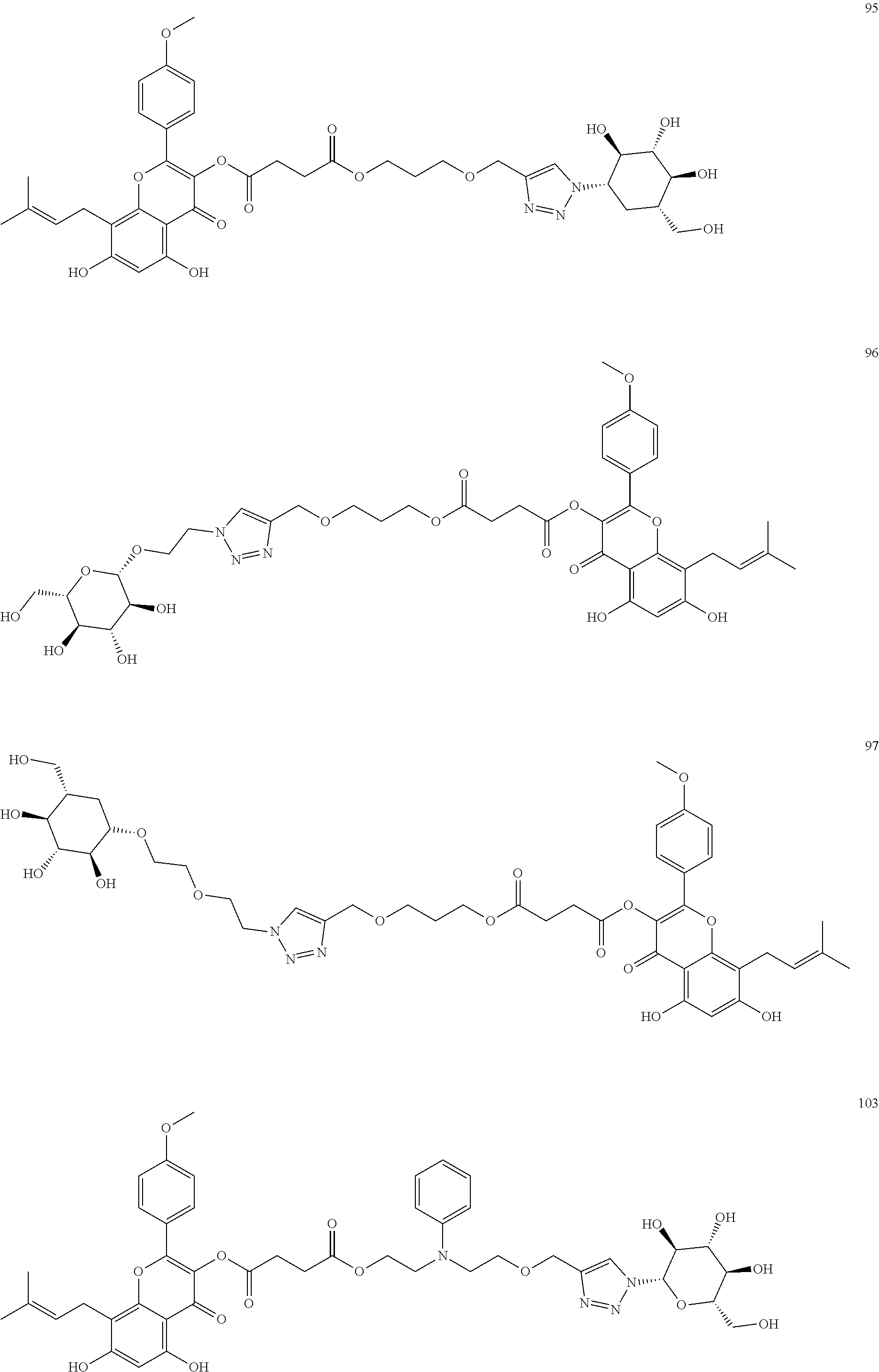

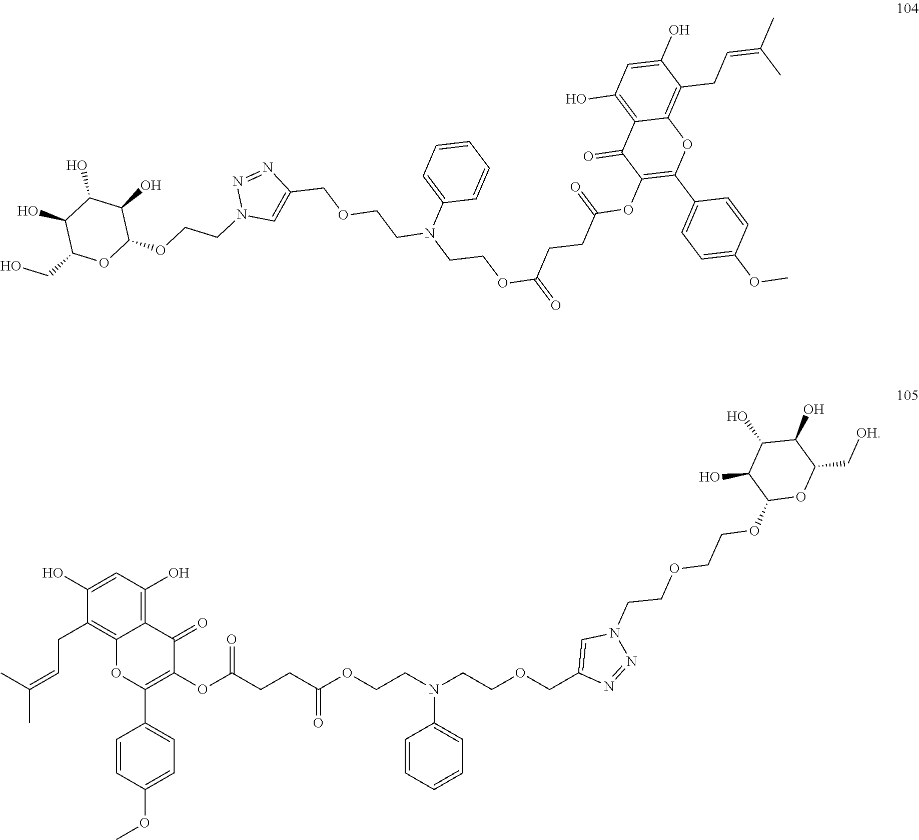

In some embodiments, the flavone derivative is one of the following compounds:

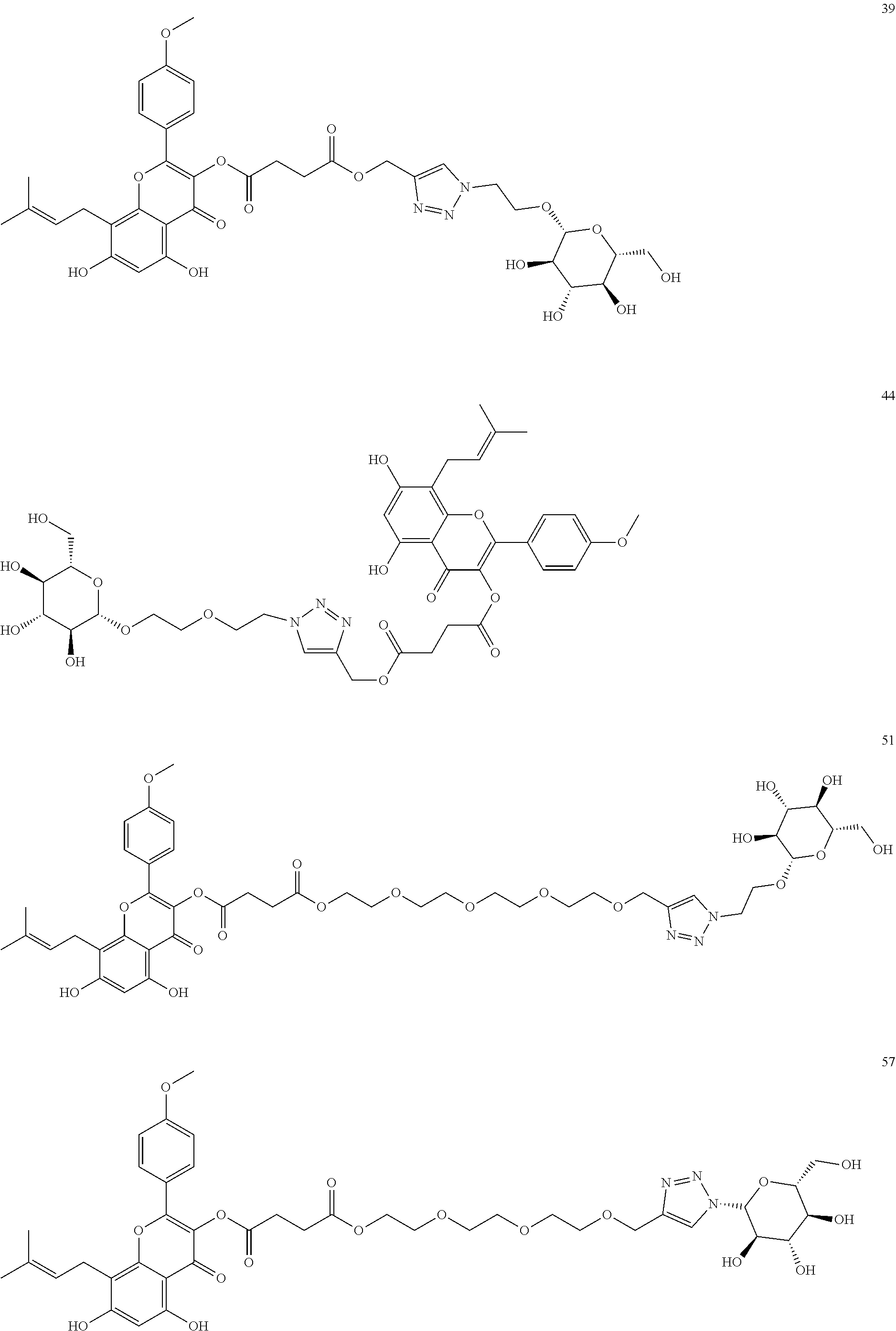

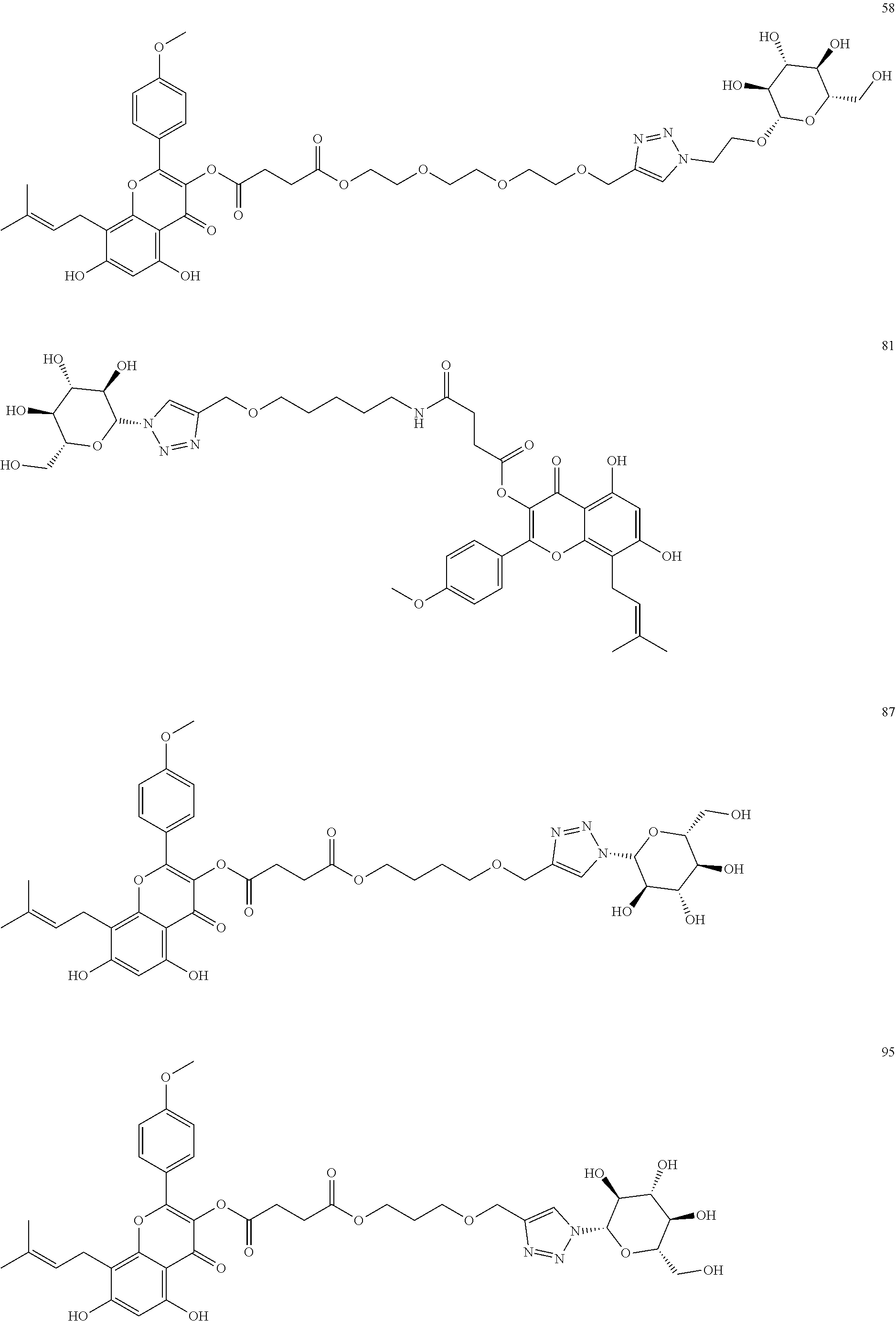

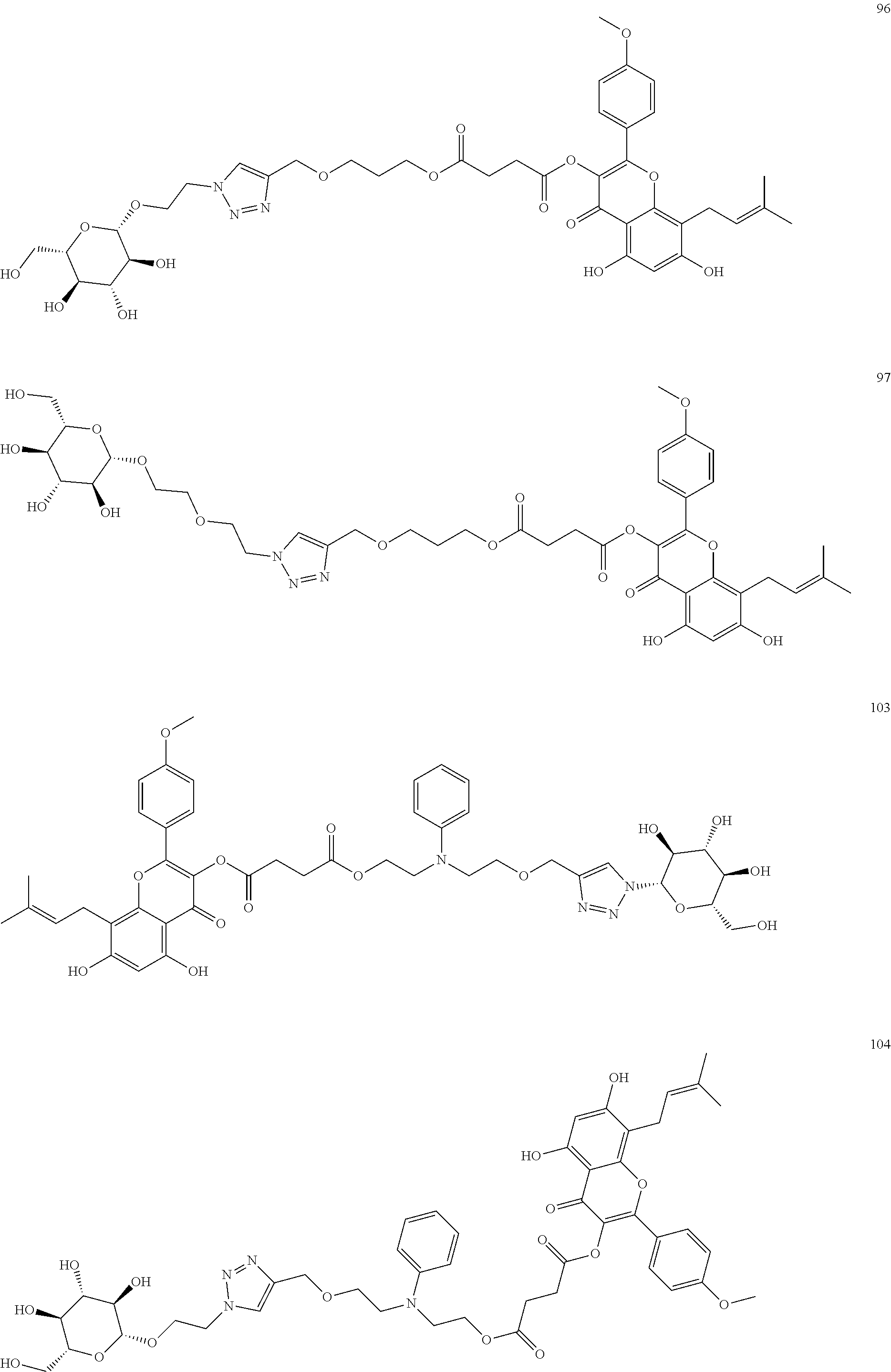

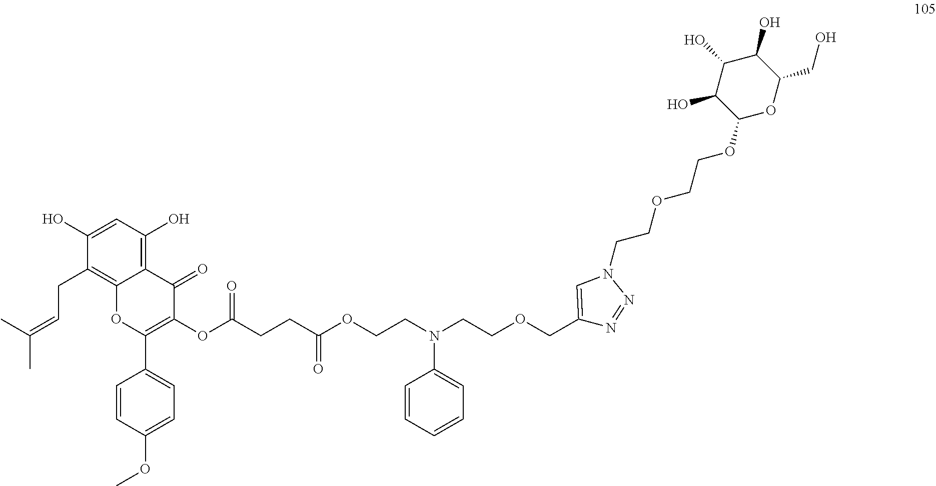 [SMILES: COc1ccc(-c2oc3c(CC=C(C)C)c(O)cc(O)c3c(=O)c2OC(=O)CCC(=O)OCCN(CCOCc2cn(CCOCCO[C@H]3O[C@@H](CO)[C@H](O)[C@@H](O)[C@@H]3O)nn2)c2ccccc2)cc1]

The second technical problem to be solved in the present disclosure is to provide an

anti-cancer drug with better solubility and anti-cancer activity, and a pharmaceutical composition thereof, health food and food.

To solve the second technical problem in the present disclosure, the anti-cancer pharmaceutical composition, health food and food comprises various crystal forms, hydrates or solvates of the flavone derivative represented by the structural formula I.

Excipients or auxiliary constituents and edible rats materials commonly used in pharmacy can be added into the anti-cancer pharmaceutical composition, health food and food.

In particular, the flavone derivative represented by the structural formula I is as below;

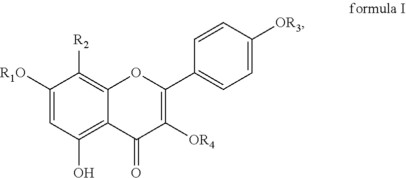

in the formula I,

R₁ represents H;

R₂ represents isopenenyl;

R₃ represents methyl; and

R₄ represents

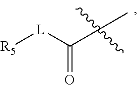

wherein R₅ is selected from the group consisting of

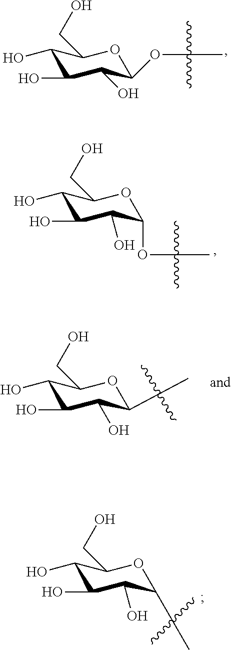

### L is

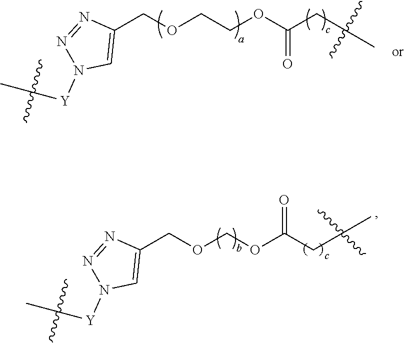

wherein, Y is

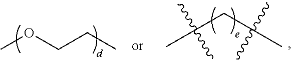

a is an integer from 0-20, b is an integer from 1-20, c is an integer from 2-6, d is an integer from 0-20, and e is an integer from 0-20.

In some embodiments, in the formula I,

R₁ represents H;

R₂ represents isopentenyl;

R₃ represents methyl; and

R₄ represents

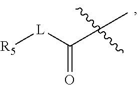

wherein R₅ is

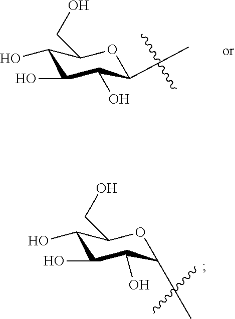

### L is

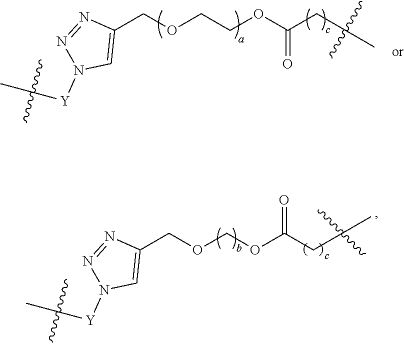

wherein, Y is

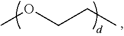

a is an integer from 0-5, b is an integer from 3-5, c is 2-4, and d is an integer from 0-4.

In further embodiments, the flavone derivative one of the following compounds:

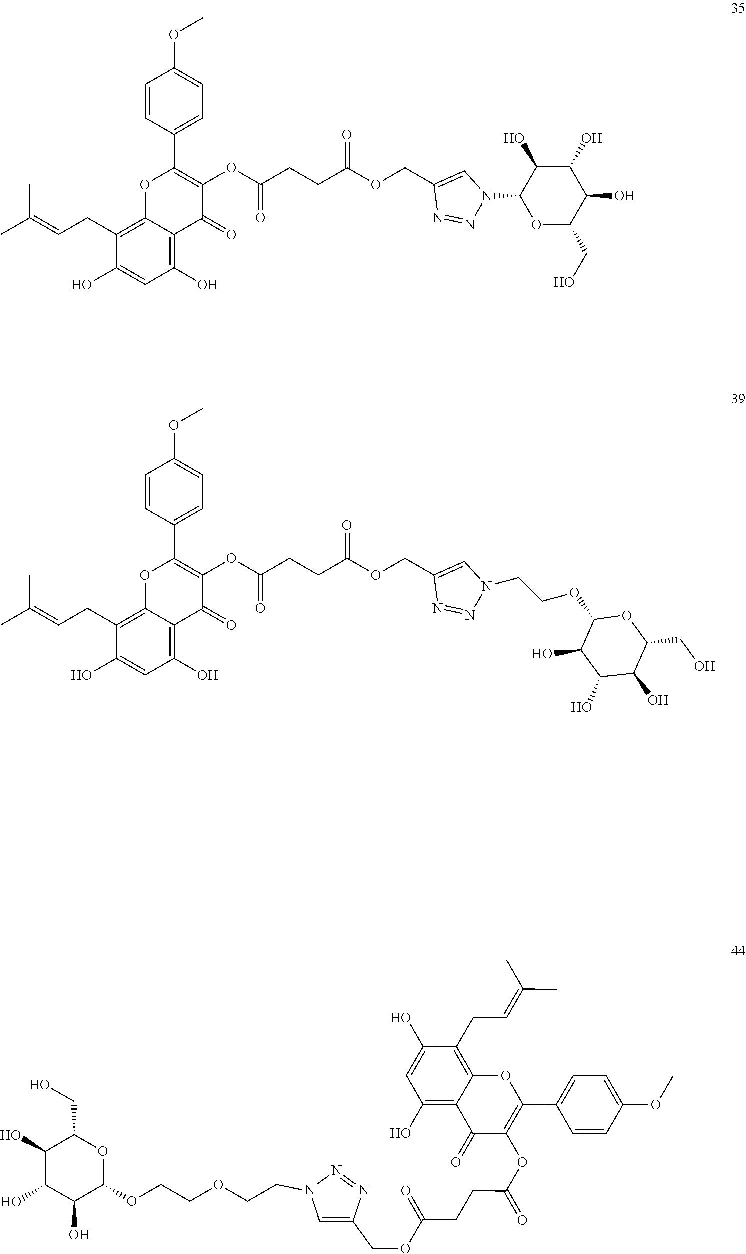 [SMILES: COc1ccc(-c2oc3c(CC=C(C)C)c(O)cc(O)c3c(=O)c2OC(=O)CCC(=O)OCc2cn([C@H]3O[C@@H](CO)[C@H](O)[C@@H](O)[C@@H]3O)nn2)cc1 ; COc1ccc(-c2oc3c(CC=C(C)C)c(O)cc(O)c3c(=O)c2OC(=O)CCC(=O)OCc2cn(CCO[C@@H]3O[C@H](CO)[C@@H](O)[C@H](O)[C@H]3O)nn2)cc1 ; COc1ccc(-c2oc3c(CC=C(C)C)c(O)cc(O)c3c(=O)c2OC(=O)CCC(=O)OCc2cn(CCOCCO[C@H]3O[C@@H](CO)[C@H](O)[C@@H](O)[C@@H]3O)nn2)cc1]

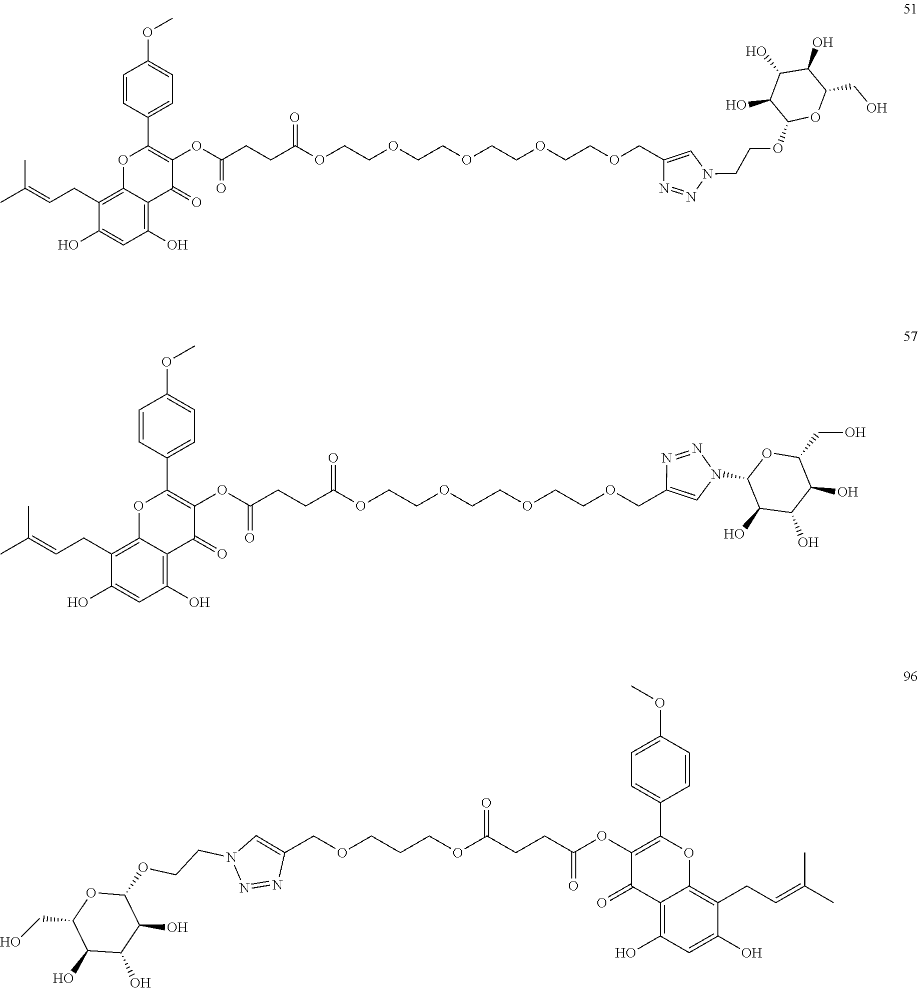 [SMILES: COc1ccc(-c2oc3c(CC=C(C)C)c(O)cc(O)c3c(=O)c2OC(=O)CCC(=O)OCCOCCOCCOCCOCc2cn(CCO[C@H]3O[C@@H](CO)[C@H](O)[C@@H](O)[C@@H]3O)nn2)cc1 ; COc1ccc(-c2oc3c(CC=C(C)C)c(O)cc(O)c3c(=O)c2OC(=O)CCC(=O)OCCOCCOCCOCc2cn([C@@H]3O[C@H](CO)[C@@H](O)[C@H](O)[C@H]3O)nn2)cc1 ; COc1ccc(-c2oc3c(CC=C(C)C)c(O)cc(O)c3c(=O)c2OC(=O)CCC(=O)OCCCOCc2cn(CCO[C@H]3O[C@@H](CO)[C@H](O)[C@@H](O)[C@@H]3O)nn2)cc1]

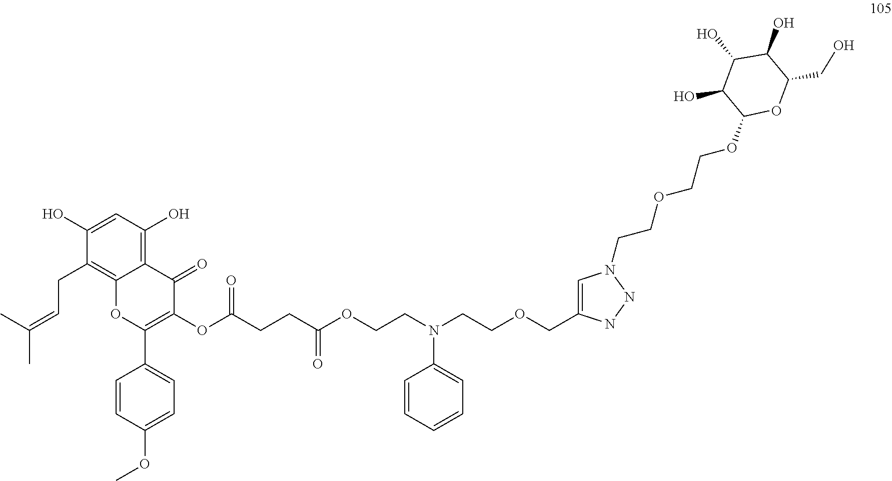 [SMILES: COc1ccc(-c2oc3c(CC=C(C)C)c(O)cc(O)c3c(=O)c2OC(=O)CCC(=O)OCCN(CCOCC2=CN(CCOCCO[C@H]3O[C@@H](CO)[C@H](O)[C@@H](O)[C@@H]3O)NN2)c2ccccc2)cc1]

To achieve the best activity and solubility at the same time, it is the most preferable that in the flavone derivative represented by the formula I:

R₁ represents H;

R₂ represents isopentenyl;

R₃ represents methyl; and

R₄ represents

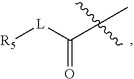

wherein R₅ is

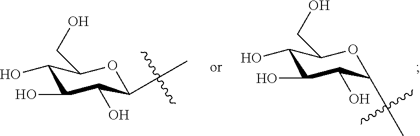

### L is

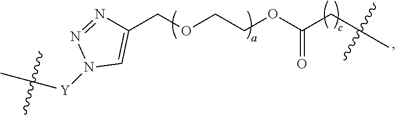

wherein Y is

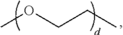

a is an integer from 1-4, c is an integer from 2-4 and d is an integer from 0-4.

In some embodiments, the flavone derivative is one of the following compounds:

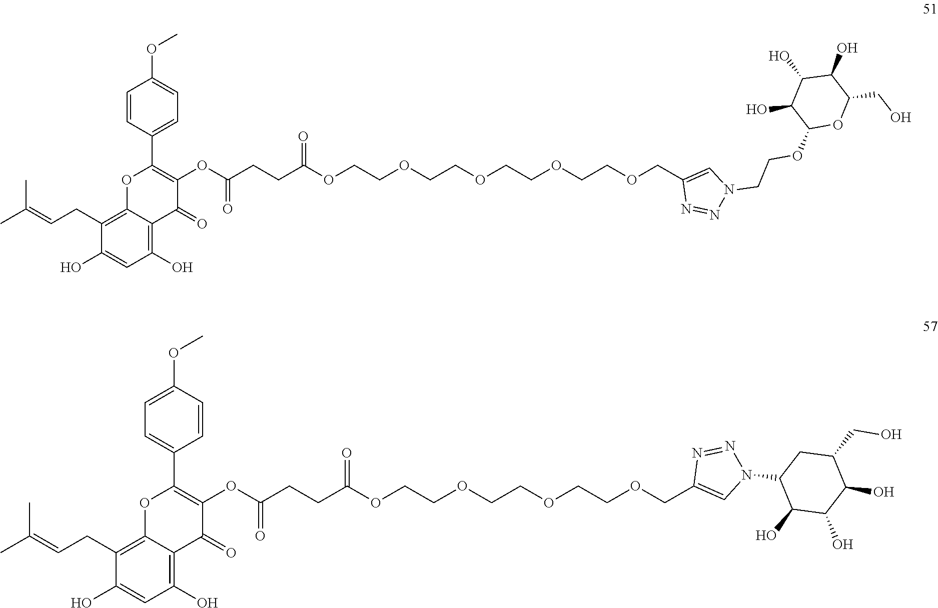

Beneficial Effects:

The present disclosure provides a class of flavone derivative with a new structure through structural modification, which has the effect of broad anti-cancer spectrum. Moreover, after structural optimization, the flavone derivative with a new structure exhibits a significant superior anti-cancer activity than that of icaritin, and at the same time has a higher solubility, thereby ensuring a better anti-tumor activity in the body.

### BRIEF DESCRIPTION OF THE DRAWINGS

FIG. 1 is a diagram showing the anti-tumor activity (n=6) in vivo, in which panel (A) is a diagram showing tumor volume over time, and panel (B) is a photograph of subcutaneous tumor.

FIG. 2 is a diagram showing body weight over time during the treatment (n=6)

### DETAILED DESCRIPTION OF THE EMBODIMENTS

The present disclosure will be further illustrated through specific examples below. However, it should be understood that these examples are only for the purpose of describing in more detail, rather than limiting the present disclosure in any way.

Materials and experimental methods used in the test are generally and specifically described in the present disclosure. Many materials and operational methods used for realizing the present disclosure are well known in the art, but they are still described in detail as much as possible in the present disclosure. It is clear to those skilled in the art that, unless otherwise specified, the materials and operational methods used hereinafter are all well known in the art.

### EXAMPLE 1

### Synthesis of Compound 35

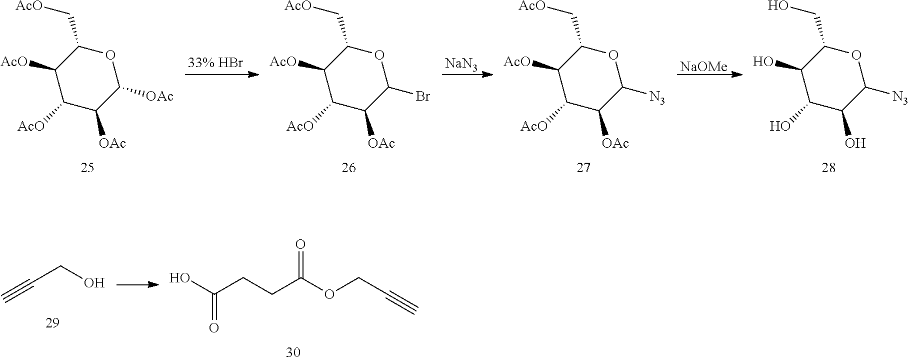

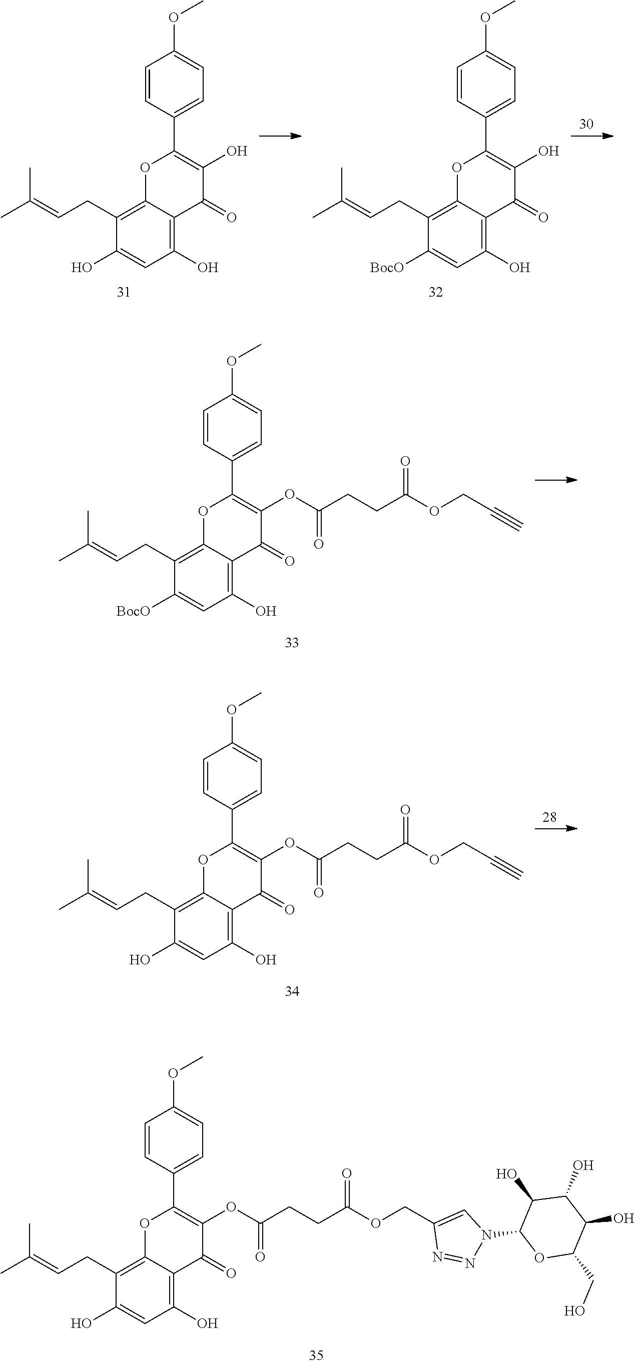

9 g of compound 25 was weighed and dissolved in 60 mL of dichloromethane, 25 mL of a 33% solution of hydrobromic acid in acetic acid was added thereto, and then the resulting mixture was subjected to a reaction at ambient temperature for 2 hours. The reaction was monitored by TLC until the reaction was completed. The resulting reactant was poured into 50 mL water, and extracted with dichloromethane (50 mL×3) to collect an organic layer. The organic layer was combined and washed once with a saturated sodium bicarbonate solution, dried over anhydrous sodium sulfate, and filtered, and then the obtained filtrate was concentrated and recrystallized with a mixed solvent of petroleum ether/ethyl acetate with a ratio of 10/1, to obtain 8.2 g of compound 26, with a yield of 86.7%.

8.2 g of the compound 26 was dissolved in 40 mL anhydrous DMSO, and 1.56 g of sodium azide was added thereto. The resulting mixture was subjected to a reaction while stirring at ambient temperature for 1 hour. The reaction was monitored by TLC until the reaction was completed. The resulting reactant was poured into 100 mL water, and extracted with ethyl acetate (50 mL×3) to collect an organic layer. The organic layer was combined and dried over anhydrous sodium sulfate and filtered, and then the obtained filtrate was concentrated and recrystallized with a mixed solvent of petroleum ether/ethyl acetate with a ratio of 10/1 , to obtain 5.8 g of compound 27, with a yield of 77.7%.

1 g of the compound 27 was weighed and dissolved in 5 mL of anhydrous methanol, and 10 mL of a 0.5 N NaOMe-MeOH solution was added thereto. The resulting mixture was stirred at ambient temperature overnight. Then, an appropriate amount of a strong-acid cation exchange resin was added, and the resulting mixture was stirred continually for 15 minutes. Under the condition that the pH of the resulting reactant was neutral or weak acid, the resulting reactant was filtered, and the obtained filtrate was concentrated to obtain 420 mg of compound 28, with a yield of 76.4%.

5 g of compound 29 was dissolved in 100 mL of anhydrous dichloromethane, and 10.26 g of succinic anhydride and 2.18 g of DMAP were added thereto. The resulting mixture was stirred at ambient temperature overnight, then added into 100 mL of water, and extracted with dichloromethane (100 mL×2) to collect an organic layer. The organic layer was combined, and washed successively with a 10% sodium bisulfate solution (100 mL×3), a 1N HCl solution (100 mL×2) and a saturated sodium chloride solution. The washed organic layer was dried over anhydrous sodium sulfate and filtered, and the obtained filtrate was concentrated, to obtain 4.5 g of compound 30 as a white solid, with a yield of 32.4%.

1 g of Icaritin (compound 31) was weighed and suspended in 50 mL of dichloromethane, and 540 mg of trimethylamine was added thereto. At this point, the reaction system became clear. 620 mg of di-tert-butyl Bicarbonate was weighed and dissolved in 20 mL of dichloromethane, which was dropwise added into the above system slowly while stirring. The resulting reactant was subjected to a reaction at ambient temperature overnight while stirring. Then the reaction was monitored by TLC (PE:EA=5:1) until the reaction was completed. The resulting reactant was concentrated, dissolved with dichloromethane and purified by a column (DCM:MeOH=100:1), to obtain 760 mg of compound 32 as a yellow solid, with a yield of 59.8%.

300 mg of the compound 32 and 100 mg of the compound 30 were weighed and dissolved in 10 mL of dichloromethane, and 159 mg of EDCI and 27.4 mg of DMAP were successively added thereto. The resulting mixture was stirred at ambient temperature overnight, concentrated at reduced pressure in vacuum, and purified by a preparation plate (PE:EA=3:1), to obtain 140 mg of compound 33 as yellow oil, with a yield of 36.1%.

140 mg of the compound 33 was dissolved in 5 mL of dichloromethane, and 1 mL of trifluoroacetic acid was added thereto. The resulting mixture was stirred at ambient temperature overnight, concentrated and purified by a preparation plate (PE:EA=3:1) to obtain 60 mg of compound 34 as yellow oil, with a yield of 51.7%.

60 mg of the compound 34 and 24 mg of the compound 28 were dissolved in 8 mL of THF-H₂O mixed solvent (1:1), and 26 mg of anhydrous cupric sulfate and 33 mg of sodium ascorbate were successively added thereto. The resulting mixture was stirred at ambient temperature overnight, concentrated and purified by column chromatography (DCM:MeOH=80:1-40:1) to obtain 34 mg of a compound 35 as a light yellow solid, with a yield of 40.3%.

¹H NMR (400 Hz, DMSO-d₆) δ11.90 (s, 1H), 8.35 (s, 1H), 7.95 (d, J=8.8 Hz, 2H), 7.17 (d, J=9.2 Hz, 2H), 6.23 (s, 1H), 5.53 (d, J=9.2 Hz, 1H), 5.37 (d, J=6.0 Hz, 1H), 5.26 (d, J=4.8 Hz, 1H), 5.17 (s, 2H), 5.13 (d, J=5.6 Hz, 1H), 4.61 (t, J=5.6 Hz, 1H), 3.87 (s, 3H), 3.66-3.76 (m, 2H), 3.37-3.44 (m, 3H), 3.19-3.25 (m, 1H), 2.95-2.99 (m, 2H), 2.87 (t, J=6.6 Hz, 2H), 2.71-2.75 (m, 2H), 1.87 (t, J=6.6 Hz, 2H), 1.35 (s, 6H).

### EXAMPLE 2

### Synthesis of Compound 39

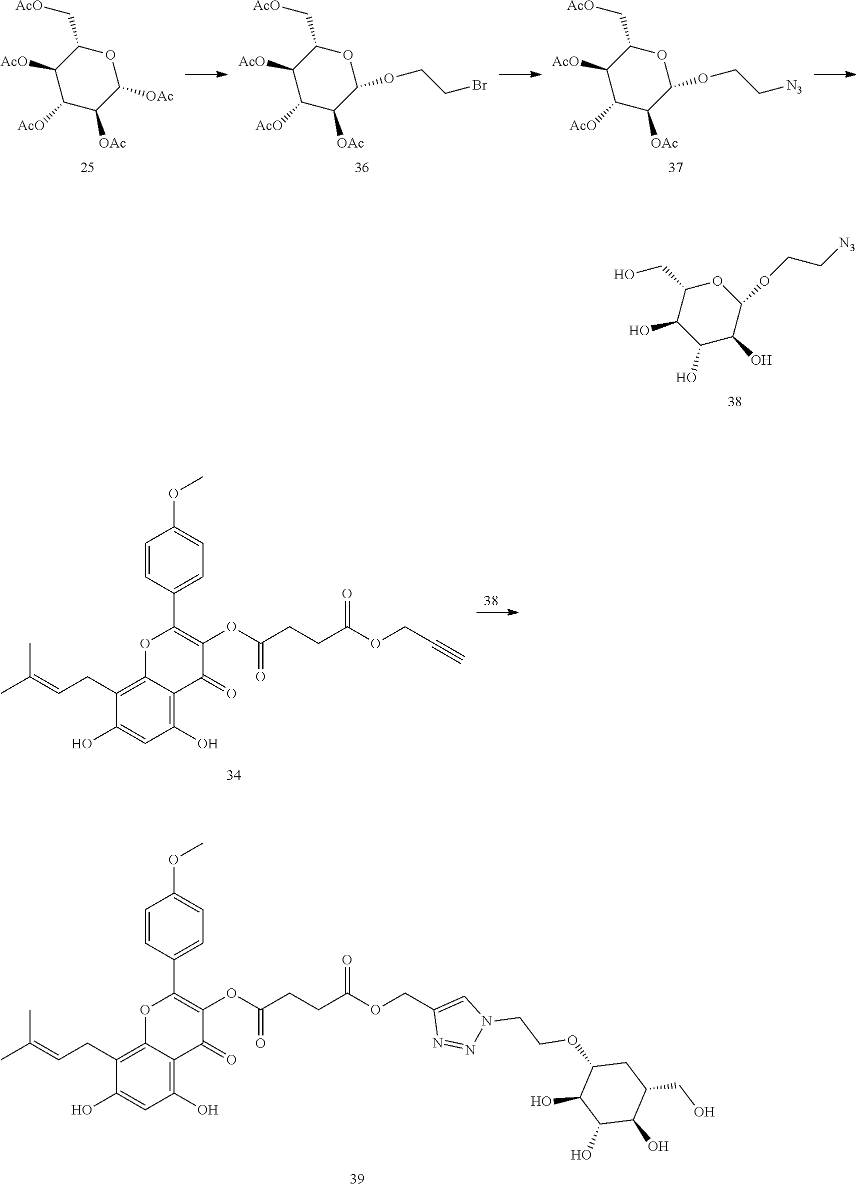 [SMILES: CC(=O)OC[C@@H]1O[C@H](OC(C)=O)[C@@H](OC(C)=O)[C@H](OC(C)=O)[C@H]1OC(C)=O ; CC(=O)OC[C@@H]1O[C@H](OCCBr)[C@@H](OC(C)=O)[C@H](OC(C)=O)[C@H]1OC(C)=O ; CC(=O)OC[C@@H]1O[C@H](OCCN=[N+]=[N-])[C@@H](OC(C)=O)[C@H](OC(C)=O)[C@H]1OC(C)=O ; [N-]=[N+]=NCCO[C@H]1O[C@@H](CO)[C@H](O)[C@@H](O)[C@@H]1O ; C#CCOC(=O)CCC(=O)Oc1c(-c2ccc(OC)cc2)oc2c(CC=C(C)C)c(O)cc(O)c2c1=O ; COc1ccc(-c2oc3c(CC=C(C)C)c(O)cc(O)c3c(=O)c2OC(=O)CCC(=O)OCc2cn(CCO[C@@H]3C[C@H](CO)[C@@H](O)[C@H](O)[C@H]3O)nn2)cc1]

18 g of compound 25 was weighed and dissolved in 150 mL of dichloromethane, and 6.3 g of bromoethanol and 8.7 mL of boron trilluoride etherate were added thereto. The resulting mixture was reacted at ambient temperature overnight. Then saturated sodium bicarbonate was added into the mixture until no gas was produced. The resulting reactant was extracted with DCM for three times to collect an organic layer. The organic layer was combined and dried, concentrated, and purified by column chromatography to obtain 10 g of compound 36, with a yield of 47.7%.

10 g of the compound 36 was weighed and dissolved in 150 mL of DMF, and 2.86 g of sodium azide was added thereto. The resulting mixture was reacted at 60° C. overnight. The reaction was monitored by TLC until the reaction was completed. The resulting reactant was cooled to ambient temperature, and added into an appropriate amount of water, extracted with ethyl acetate twice (100 mL×3), and concentrated to remove a few layers of residual DMF in a small amount. An appropriate amount of ethanol was added to resolve solid, which was filtered to obtain 6.9 g of the product compound 37, with a yield of 75.2%.

1 g of the compound 37 was weighed and dissolved in 5 mL of anhydrous methanol, and 10 mL of 0.5N NaOMe-Me0H solution was added thereto. The resulting mixture was stirred at ambient temperature overnight. Then an appropriate amount of strong-acid cation exchange resin was added into the mixture and stirred continually for another 15 minutes. Under the condition that the pH of the resulting reactant was neutral or weak acid, the resulting reactant was filtered, and the obtained filtrate was concentrated to obtain 430 mg of compound 38, with a yield of 72.0%.

40 mg of the compound 34 and 20 mg of the compound 38 were dissolved in 10 mL of THF-H₂O mixed solvent (1:1), and 13 mg of anhydrous cupric sulfate and 16 mg of sodium ascorbate were sucessively added thereto. The resulting mixture was stirred at ambient temperature overnight, concentrated and purified by a flash column (DCM:MeOH=8:1) to obtain a crude product. The crude product was purified by a preparation plate (DCM:MeOH=8:1) to obtain 25 mg of compound 39 as a light yellow solid, with a yield of 41.6%.

¹H NMR (400 Hz, CD₃OD) δ8.09 (s, 1H), 7.87 (d, J=9.2 Hz, 2H), 7.07 (d, J=9.2 Hz, 2H), 6.16 (s, 1H), 5.21 (s, 2H), 4.60 (d, J=5.0 Hz, 2H), 4.27 (d, J=8.0 Hz, 1H), 4.19-4.22 (m, 1H), 3.96-4.00 (m, 1H), 3.83-3.87 (m, 4H 3.61-3.66 (m, 1H), 3.24-3.26 (m, 2H), 3.14-3.18 (m, 1H), 2.97-3.00 (m, 2H), 2.86 (t, J=6.8 Hz, 2H), 2.74-2.77 (m, 2H), 1.90 (t, J=6.8 Hz, 2H), 1.37 (s, 6H).

### EXAMPLE 3

### Synthesis of Compound 44

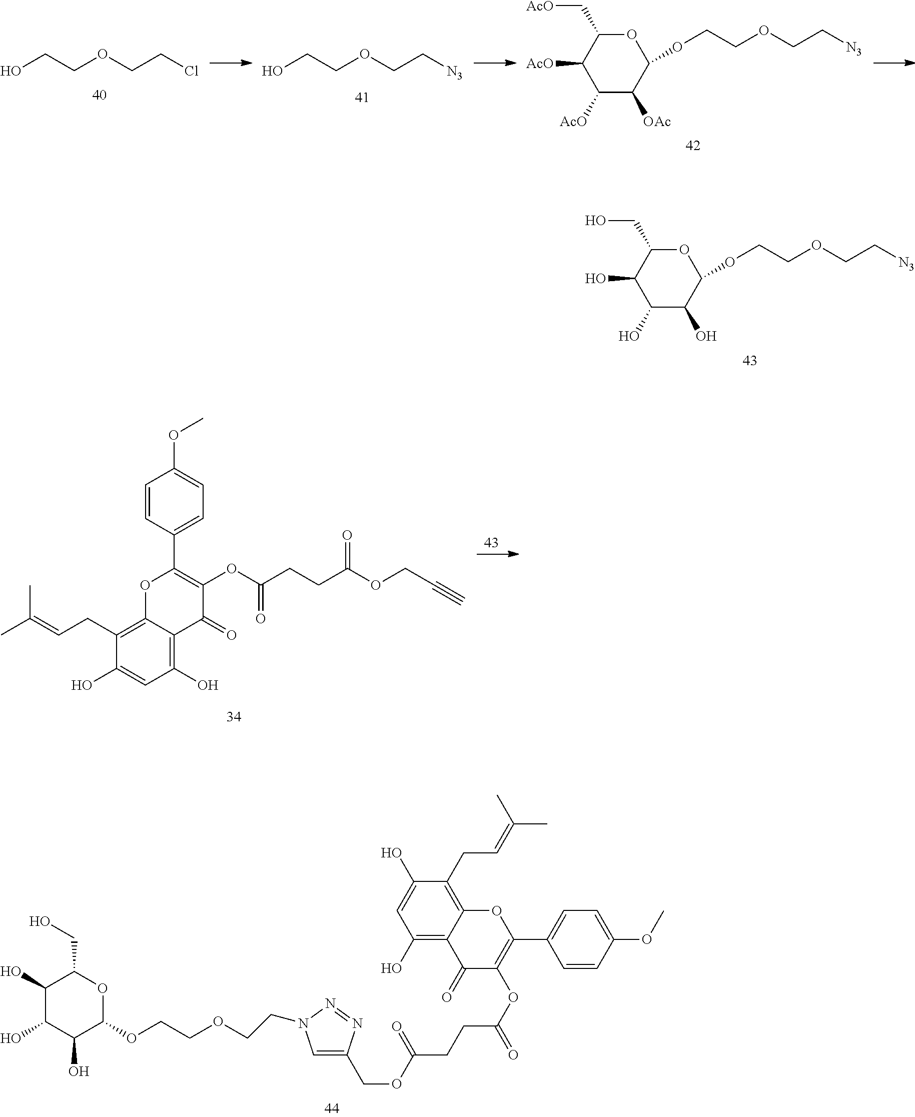 [SMILES: OCCOCCCl ; [N-]=[N+]=NCCOCCO ; CC(=O)OC[C@@H]1O[C@H](OCCOCCN=[N+]=[N-])[C@@H](OC(C)=O)[C@H](OC(C)=O)[C@H]1OC(C)=O ; [N-]=[N+]=NCCOCCO[C@H]1O[C@@H](CO)[C@H](O)[C@@H](O)[C@@H]1O ; C#CCOC(=O)CCC(=O)Oc1c(-c2ccc(OC)cc2)oc2c(CC=C(C)C)c(O)cc(O)c2c1=O ; COc1ccc(-c2oc3c(CC=C(C)C)c(O)cc(O)c3c(=O)c2OC(=O)CCC(=O)OCc2cn(CCOCCO[C@H]3O[C@@H](CO)[C@H](O)[C@@H](O)[C@@H]3O)nn2)cc1]

1.24 g of compound 40 was weighed into 15 mL of water, and 1.95 g of sodium azide and 150 mg of sodium iodide were successively added thereto. The resulting mixture was subjected to a reaction at 60° C. for 96 hours, cooled to ambient temperature, saturated with sodium chloride, and extracted with dichloromethane (20 mL×3) to collect an organic layer. The organic layer was combined, dried, and concentrated to obtain 900 mg of compound 41 as a colorless liquid, with a yield of 68.7%.

1.25 g of compound 25 and 524 mg of the compound 41 were weighed and dissolved in 20 mL of dichloromethane. The mixture was cooled to 0° C., and 0.6 mL of boron trifluoride etherate was added thereto slowly. The resulting mixture was heated to ambient temperature slowly and reacted at ambient temperature overnight, and then 1 mL of triethylarnine was added to the reaction system drop rise slowly. The resulting reactant was concentrated at reduced pressure in vacuum, and purified by a column (PE:EA=20:1-2:1) to obtain 1.03 g of compound 42, with a yield of 70%.

1 g of the compound 42 was weighed and dissolved in 5 mL of anhydrous methanol, and 10 mL of 0.5N NaOMe-MeOH solution was added thereto. The resulting mixture was stirred at ambient temperature overnight. Then an appropriate amount of strong-acid cation exchange resin was added into the mixture and stirred continually for another 15 minutes. Under the condition that the pH of the resulting reactant was neutral or weak acid, the resulting reactant was filtered, and the obtained filtrate was concentrated to obtain 400 mg of compound 43, with a yield of 63.0%.

40 mg of the compound 34 and 23 mg of the compound 43 were dissolved in 10 mL of THF-H₂O mixed solvent (1:1), and 13 mg of anhydrous cupric sulfate and 16 mg of sodium ascorbate were added successively. The resulting mixture was stirred at ambient temperature overnight, concentrated and then purified by a flash column (DCM:Me0H — :1) to obtain a crude product. The crude product was purified by a preparation plate (DCM:MeOH=8:1) to obtain 30 mg of compound 44 as a light yellow solid, with a yield of 47.6%.

¹H NMR (400 Hz, CD₃ OD) δ 8.04 (s, 1H), 7.88 (d, J=8.8 Hz, 2H), 7.07 (d, J=8.8 Hz, 2H), 6.16 (s, 1H), 5.22 (s, 2H), 4.54 (t, J=5.0 Hz, 2H), 4.25 (d, J=7.6 Hz, 1H), 3.82-3.92(m, 7H), 3.61-3.68 (m, 4H), 3.34-3.36 (m, 1H) 3.25-3.26 (m , 1H), 3.15-3.19 (m, 1H), 2.97-3.00 (m, 2H), 2.87 (t, J=6.6 Hz, 2H), 2.75-2.78 (m, 2H), 1.90 (t, J=6.8 Hz, 2H), 1.37 (s, 6H).

### EXAMPLE 4

### Synthesis of Compound 50

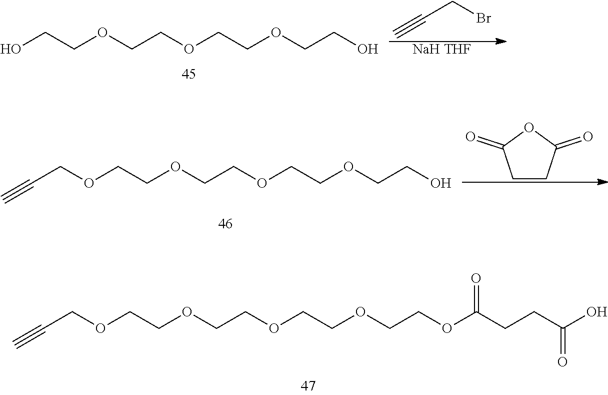

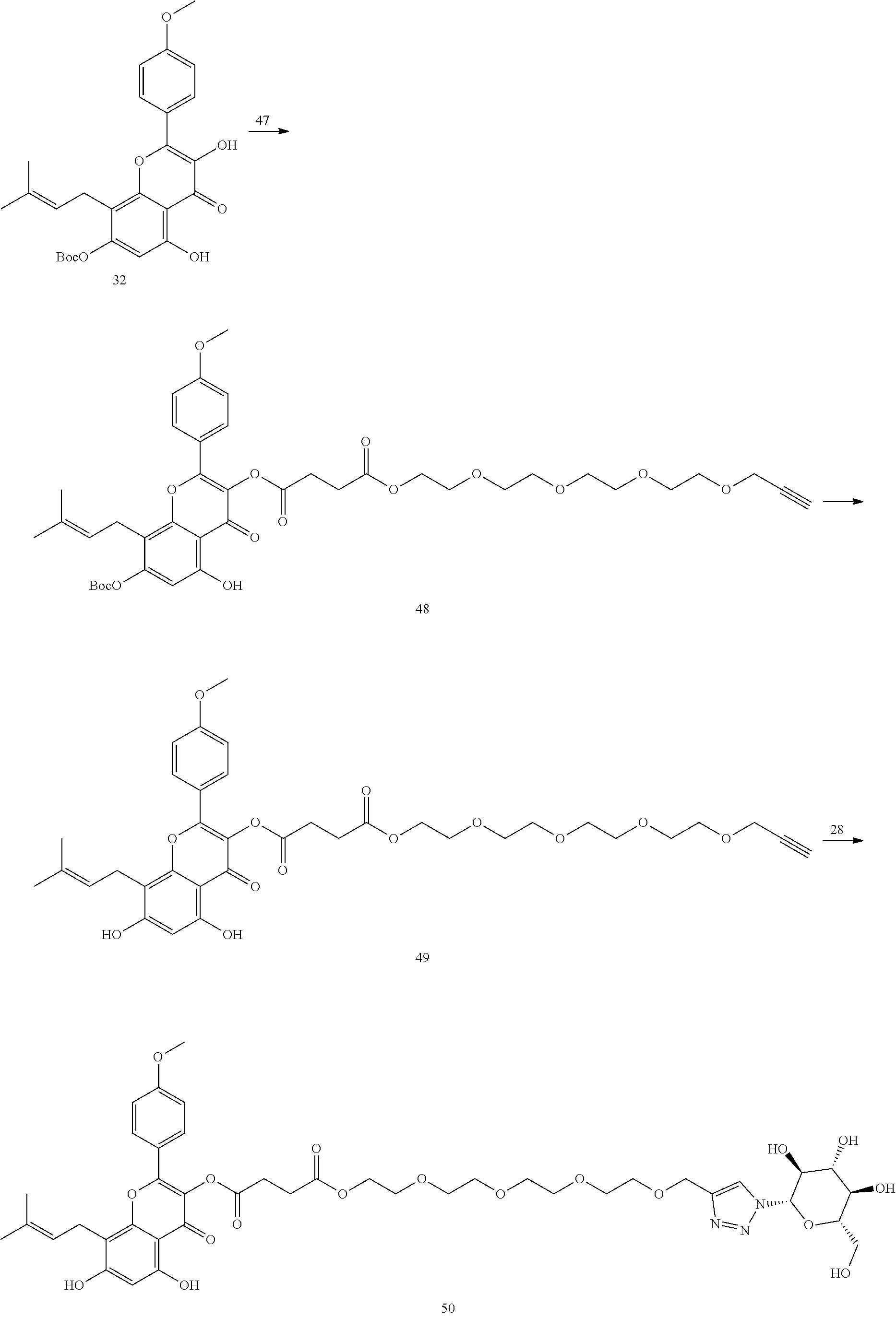

80 mL of tetraglycol (compound 45) was dissolved in 100 mL of anhydrous tetrahydrofuran, and the mixture was cooled to 0° C. Then 5.5 g of sodium hydride was added into the mixture in small batches. The resulting mixture was subjected to a reaction at this temperature for 2 hours. 10 g of 3-bromopropyne was weighed and dissolved in 60 mL of anhydrous tetrahydrofuran, and then the obtained solution was dropwise added into the above reaction slowly. The resulting reactant was reacted at ambient temperature overnight. 40 mL of water was added to the resulting reactant. The resulting reactant was concentrated to remove tetrahydrofuran, and extracted with ethyl acetate (100 mL×3) to collect an organic layer. The organic layer was combined, dried over anhydrous sodium sulfate and filtered. The obtained filtrate was concentrated and purified by column chromatography to obtain 5 g of compound 46 as a light yellow liquid, with a yield of 25.2%.

1 g of the compound 46 was dissolved in 20 mL of anhydrous dichloromethane, and 646 mg of succinic anhydride and 53 mg of DMAP were added thereto. The resulting mixture was stirred at 36° C. overnight, concentrated and purified by a column (DCM:MeOH=100:1-50:1) to obtain 720 mg of compound 47 as a light yellow liquid, with a yield of 50.3%

300 mg of the compound 32 and 212 mg of the compound 47 were weighed and dissolved in 10 mL of dichloromethane, and 159 mg of EDCI and 27.4 mg of DMAP were added successively thereto. The resulting mixture was stirred at ambient temperature overnight, concentrated at reduced pressure in vacuum, and purified by a preparation plate (PE:EA=2:1) to obtain 130 mg of compound 48 as yellow oil, with a yield of 25.9%.

130 mg of the compound 48 was dissolved in 5 mL of dichloromethane, and 1 mL of trifluoroacetic acid was added thereto. The resulting mixture was stirred at ambient temperature overnight, concentrated and purified by a preparation plate (PE:EA=2:1) to obtain 70 mg of compound 49 as yellow oil, with a yield of 61.9%.

35 mg of the compound 49 and 11 mg of the compound 28 were dissolved in 8 mL of THF-H₂O mixed solvent (1:1), and 13 mg of anhydrous cupric sulfate and 16 mg of sodium ascorbate were added successively thereto. The resulting mixture was stirred at arribient temperature overnight, concentrated and purified by a flash column (DCM:MeOH=8:1.) to obtain a crude product. The crude product was purified by a preparation plate (DCM:MeOH=8:1) to obtain 15 mg of compound 50 as a light yellow solid, with a yield of 32.9%.

¹H NMR (400 Hz, DMSO-d₆) δ 12.08 (s, 1H), 10.96 (s, 1H), 8.27 (s, 1H), 7.87 (d, J=9.2 Hz, 2H), 7.15 (d, J=9.2 Hz, 2H), 6.35 1H), 5.51 (d, J=9.2 Hz, 1H), 5.35 (d, J=6.0 Hz, 1H), 5.25 (d, J=4.8 Hz, 1H), 5.14-5.19 (m, 1H), 5.13 (d, J=4.8 Hz, 1H), 4.60-4.65 (m, 1H), 4.52 (s, 2H), 4.12-4.14 (m, 2H), 3.87 (s, 3H), 3.68-3.76 (m, 3H), 3.55-3.59 (m, 4H), 3.48-3.51 (m, 9H), 3.38-3.44 (m, 5H), 3.22-3.25 (m, 1H), 2.91-2.94 (m, 2H), 2.68-2.71 (m, 2H), 1.70 (s, 3H), 1.63 (s, 3H).

### EXAMPLE 5

### Synthesis of Compound 51

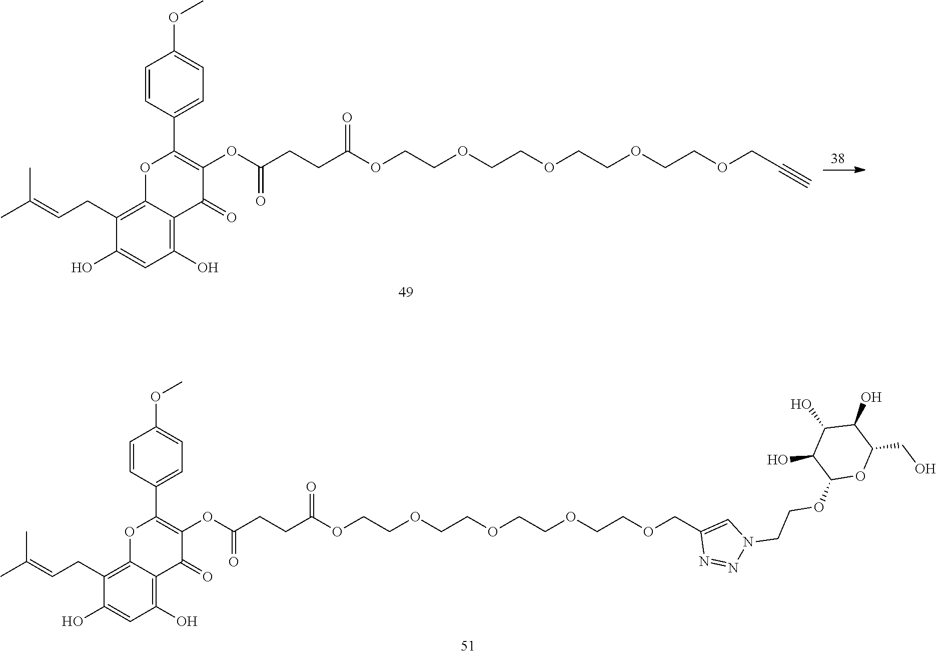

35 mg of the compound 49 and 13 mg of the compound 38 were dissolved in 8 mL of THF-H₂O mixed solvent (1:1), and 13 mg of anhydrous cupric sulfate and 16 mg of sodium ascorbate were added successively thereto. The resulting mixture was stirred at ambient temperature overnight, concentrated and purified by a flash column (DCM:MeOH=8:1) to obtain a crude product. The crude product was purified by a preparation plate (DCM:MeOH=8:1) to obtain 18 mg of compound 51 as a light yellow solid, with a yield of 37.7%.

¹H NMR (400 Hz, CD₃OD) δ 8.07 (s, 1H), 7.92 (d, J=9.2 Hz, 2H), 7.11 (d, J=8.8 Hz, 2H), 6.18 (s, 1H), 4.60-4.63 (m, 4H), 4.28 (d, J=8.0 Hz, 1H), 4.20-4.22 (m, 31-1), 3.96-4.01 (m, 1H), 3.89 (s, 3H), 3.84 (d, J=11.6 Hz, 1H), 3.65-3.67 (m, 3H), 3.58-3.62 (m, 11H), 3.32-3.35 (m, 1H), 3.24-3.26 (m, 2H), 3.16 (t, J=8.4 Hz, 1H), 2.99 (t, J=6.6 Hz, 2H), 2.90 (t, J=6.6 Hz, 2H), 2.73-2.76 (m, 2H), 1.91 (t, J=6.6 Hz, 2H), 1.38 (s, 6H)

### EXAMPLE 6

### Synthesis of Compound 57

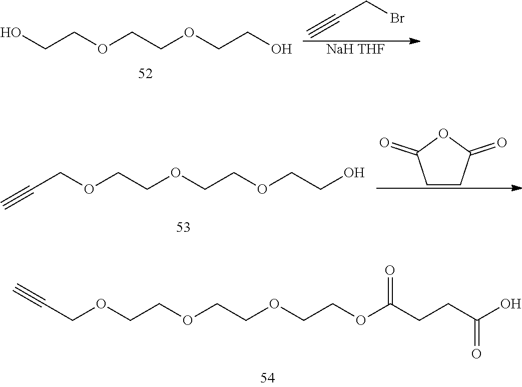

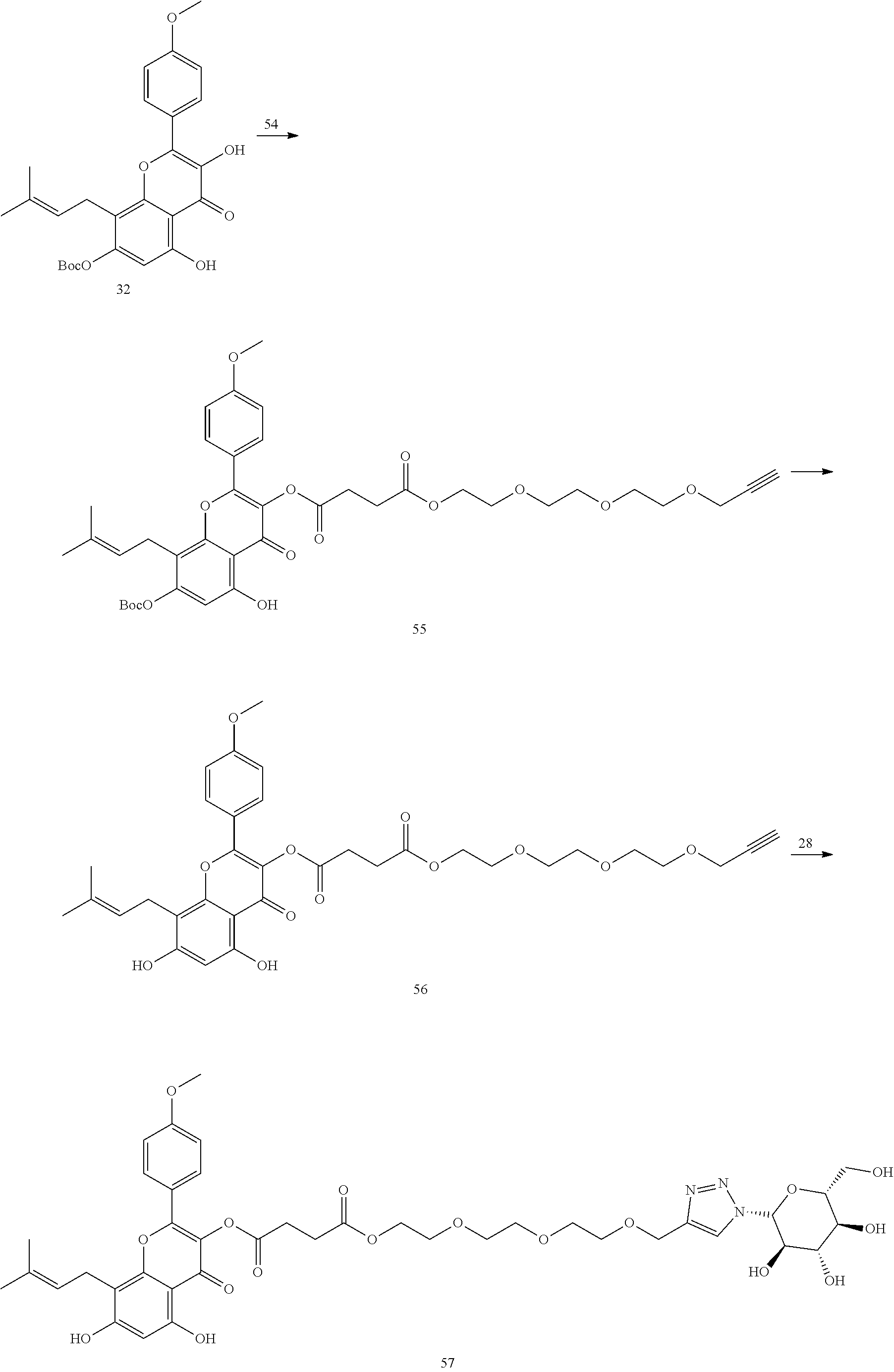

65 mL, of triglycol (compound 52) was dissolved in 100 mL, of anhydrous tetrahydrofuran, and the mixture was cooled to 0° C. Then 5.5 g of sodium hydride was added into the mixture in small batches. The resulting mixture was subjected to a reaction at this temperature for 2 hours. 10 g of 3-bromopropyne was weighed and dissolved in 60 mL of anhydrous tetrahydrofuran, and then the obtained solution was dropwise added into the above reaction slowly. The resulting reactant was reacted at arribient temperature overnight. 40 mL of water was added to the resulting reactant. The resulting reactant was concentrated to remove tetrahydrofuran and extracted with ethyl acetate (100 mL×3) to collect an organic layer. The organic layer was combined, dried over anhydrous sodium sulfate, and filtered. The obtained filtrate was concentrated and purified by column chromatography to obtain 7.7 g of compound 53 as a light yellow liquid, with a yield of 48.4%.

1 g of the compound 53 was dissolved in 20 mL of anhydrous dichloromethane, and 798 mg of succinic anhydride and 65 mg of DMAP were added thereto. The resulting mixture was stirred at 36° C. overnight, concentrated and purified by a column (DCM:MeOH=100:1-50:1) to obtain 700 mg of compound 54 as a light yellow liquid, with a yield of 45.8%.

300 mg of the compound 32 and 185 mg of the compound 54 were weighed and dissolved in 10 mL of dichloromethane, and 159 mg of EDCI and 27.4 mg of DMAP were added successively thereto. The resulting mixture was stirred at ambient temperature overnight, concentrated at reduced pressure in vacuum, and purified by a preparation plate (PE:EA=2:1) to obtain 135 mg of compound 55 as yellow oil, with a yield of 28.5%,

135 mg of the compound 55 was dissolved in 5 mL of dichloromethane, and 1 mL of trifluoroacetic acid was added thereto. The resulting mixture was stirred at ambient temperature overnight, concentrated and purified by a preparation plate (PE:EA=2:1) to obtain 70 mg of compound 56 as yellow oil, with a yield of 59.8%.

35 mg of the compound 56 and 11 mg of the compound 28 were dissolved in 8 mL of THF-H₂O mixed solvent (1:1), and 13 mg of anhydrous cupric sulfate and 16 mg of sodium ascorbate were added successively thereto. The resulting mixture was stirred at ambient temperature overnight, concentrated and purified by a flash column (DCM:MeOH=8:1) to obtain a crude product. The crude product was purified by a preparation plate (DCM:MeOH=8:1) to obtain 20 mg of compound 57 as a light yellow solid, with a yield of 43.5%.

¹H NMR (400 Hz, CD₃OD) δ 8.17 (s, 1H), 7.90 (d, J=9.2 Hz, 2H), 7.08 (d, J=9.2 Hz, 2H), 6.16 (s, 1H), 5.58 (d, J=9.2 Hz, 1H), 4.62 (s, 2H), 4.20-4.23 (m, 2H), 3.85-3.90 (m, 5H), 3.48-3.61 (m, 13H), 2.97 (t, J=6.6 Hz, 2H), 2.87 (t, =6.8 Hz, 2H), 2.73 (t, J=6.6 Hz, 2H), 1.90 (t, J=6.6 Hz, 2H), 1.37 (s, 6H).

### EXAMPLE 7

### Synthesis of Compound 58

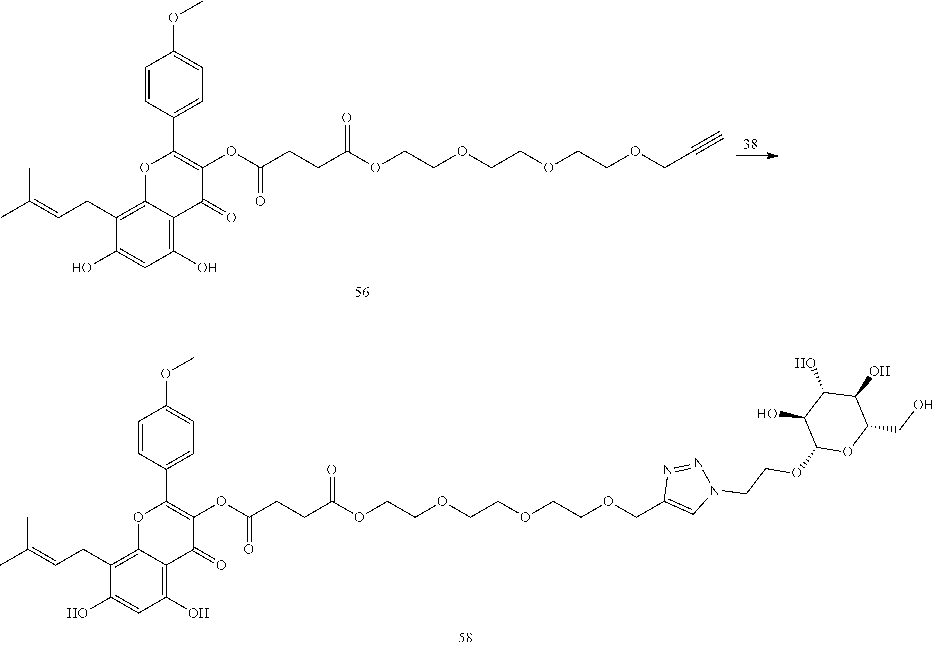

35 mg of the compound 56 and 12 mg of the compound 38 were dissolved in 8 mL THF-1-H₂O mixed solvent (1:1), and 13 mg of anhydrous cupric sulfate and 16 mg of sodium ascorbate were added successively thereto. The resulting mixture was stirred at ambient temperature overnight, concentrated and purified by a flash column (DCM:MeOH=8:1) to obtain a crude product. The crude product was purified by a preparation plate (DCM:MeOH=8:1) to obtain 15 mg of compound 58 as a light yellow solid, with a yield of 30.1%.

¹H NMR (400 Hz, CD₂OD) δ 8.06 (s, 1H), 7,89 (d, J=8.8 Hz, 214), 7.07 (d, J=8.8 Hz, 2H), 6.16 (s, 1H), 4.59-4.62 (m, 4H), 4.28 (d, J=7.6 Hz, 1H), 4.19-4.23 (m, 3H), 3.97-4.00 (m, 1H), 3.83-3.87 (m, 4H), 3.57-3.67 (m, 11H), 3.25-3.26 (m, 2H), 3.14-3.18 (m, 1H), 2.96-2.99 (m, 2H), 2.86 (t, J=6.6 Hz, 2H), 2.72-2.76 (m, 2H), 1.89 (t, J=6.8 Hz, 2H), 1.37 (s, 6H).

### EXAMPLE 8

### Synthesis of Compound 64

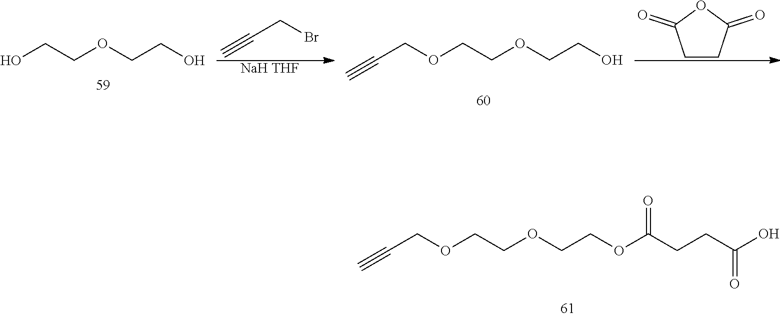

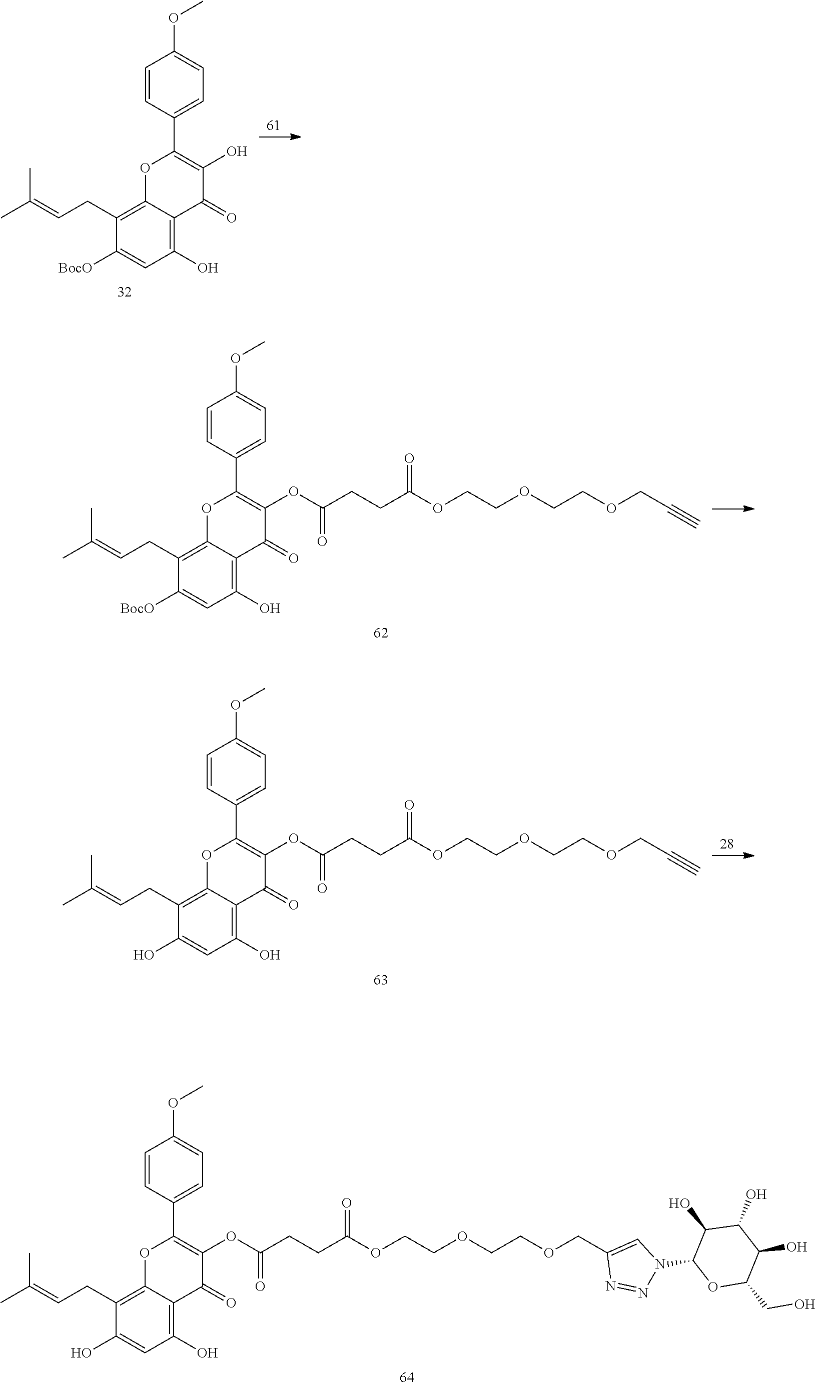

40 mL of diglycol (compound 59) was dissolved in 100 mL of anhydrous tetrahydrofuran, and the mixture was cooled to 0° C. Then 6 g of sodium hydride was added into the mixture in small batches. The resulting mixture was subjected to a reaction at this temperature for 2 hours. 10 g of 3-bromopropyne was weighed and dissolved in 60 mL of anhydrous tetrahydrofuran, and then the obtained solution was dropwise added into the above reaction slowly. The resulting reactant was reacted at ambient temperature overnight. 40 mL of water was added to the resulting reactant. The resulting reactant was concentrated to remove tetrahydrofuran, and extracted with ethyl acetate (100 mL×3) to collect ail organic, layer. The organic layer was combined, dried over anhydrous sodium sulfate, and filtered. The obtained filtrate was concentrated and purified by column chromatography to obtain 7 g of compound 60 as a light yellow liquid, with a yield of 57.3%.

1 g of the compound 60 was dissolved in 20 mL of anhydrous dichloromethane, and 1.04 g of succinic anhydride and 85 mg of DMAP were added thereto. The resulting mixture was stirred at 36° C. overnight, concentrated and purified by a column (DCM:MeOH=100:1-50:1) to obtain 800 mg of compound 61 as a light yellow liquid, with a yield of 47.3%.

300 mg of the compound 32 and 156 mg of the compound 61 were weighed and dissolved in 10 mL of dichloromethane, and 159 mg of EDCI and 27.4 mg of DMAP were added successively thereto. The resulting mixture was stirred at ambient temperature overnight, concentrated at reduced pressure in vacuum, and purified by a preparation plate (PE:EA=2:1) to obtain 12( )mg of compound 62 as yellow oil, with a yield of 27.0%.

120 mg of the compound 62 was dissolved in 5 mL dichloromethane, and 1 mL of trifluoroacetic acid was added thereto. The obtained mixture was stirred at ambient temperature overnight, concentrated and purified by a preparation plate (PE:EA=2:1) to obtain 60 mg of compound 63 as yellow oil, with a yield of 58.2%.

30 mg of the compound 63 and 11 mg of the compound 28 were dissolved in 8 mL of THF-H₂O mixed solvent (1:1), and 13 mg of anhydrous cupric sulfate and 16 mg of sodium ascorbate were added successively thereto. The resulting mixture as stirred at ambient temperature overnight, concentrated and purified by a flash column (DCM:MeOH=8:1) to obtain a crude product. The crude product was purified by a preparation plate (DCM:MeOH=8:1) to obtain 14 mg of compound 64 as a light yellow solid, with a yield of 35.0%.

¹H NMR (400 Hz, CD₃OD) δ 8.15 (s, 1H), 7.90 (d, J=8.8 Hz, 2H), 7.09 (d, J=8.8 Hz, 2H), 6.17 (s, 1H), 5.58 (d, J=9.2 Hz, 1H), 4.62 (s, 2H), 4.20-4.22 (m. 2H), 3.85-3.90 (m, 5H), 3.63-3.66 (m. 6H), 3.49-3.57 (m, 3H), 2.98 (t, J=6.6 Hz, 2H), 2.88 (t, J=6.6 Hz, 2H), 2.74 (t, J=6.6 Hz, 2H), 1.90 (t, J=6.6 Hz, 2H), 1.37 (s, 6H).

### EXAMPLE 9

### Synthesis of Compound 65

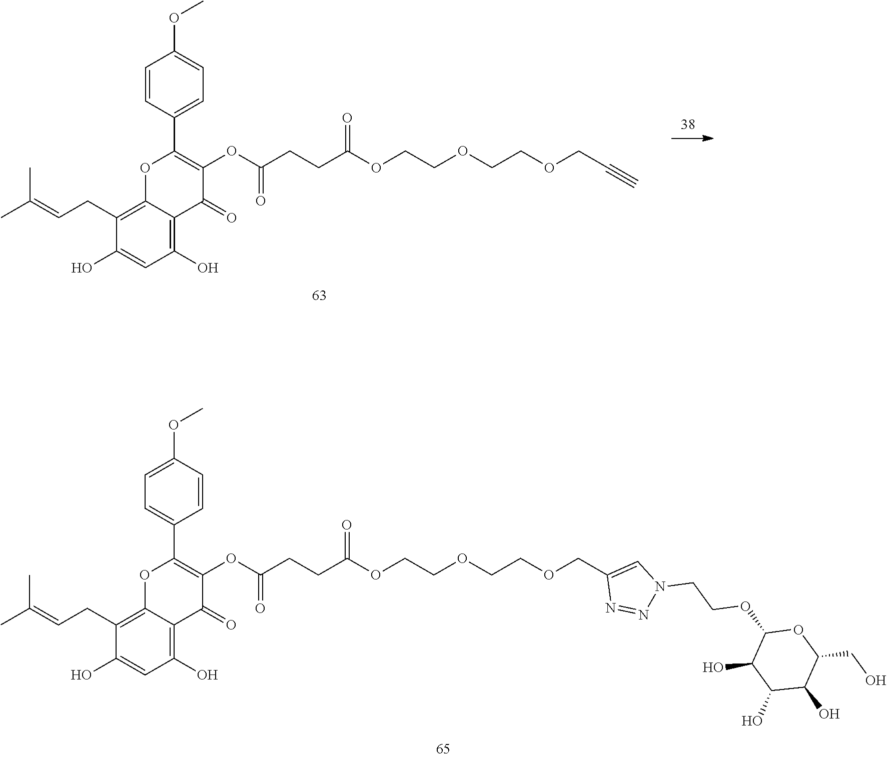

30 mg the compound 63 and 12 mg the compound 38 were dissolved in 8 mL THF-H₂O mixed solvent (1:1), and 13 mg of anhydrous cupric sulfate and 16 mg of sodium ascorbate were added successively thereto. The resulting mixture was stirred at ambient temperature overnight, concentrated and purified by a flash column (DCM:MeOH=8:1) to obtain a crude product. The crude product was purified by a preparation plate (DCM:MeOH=8:1) to obtain 16 mg of compound 65 as a light yellow solid, with a yield of 37.6%.

¹H NMR (400 Hz, CD₃OD) δ 8.05 (s, 1H), 7.91 (d, J=8.8 Hz, 2H), 7.10 (d, J=9.2 Hz, 2H), 6.18 (s, 1H), 4.58-4.61 (m, 4H), 4.28 (d, J=8.0 Hz, 1H), 4.20-4.23 (m, 3H), 3.95-4.00 (m, 1H), 3.82-3.87 (m, 4H), 3.62-3.66 (m, 7H), 3.24-3.26 (m, 2H), 3.16 (t, J=8.4 Hz, 1H), 2.98 (t, J=6.6 Hz, 2H), 2.89 (t, J=6.8 Hz, 2H), 2.75 (t, J=6.6 Hz, 2H), 1.91 (t, J=6.6 Hz, 2H), 1.38 (s. 6H).

### EXAMPLE 10

### Synthesis of Compound 71

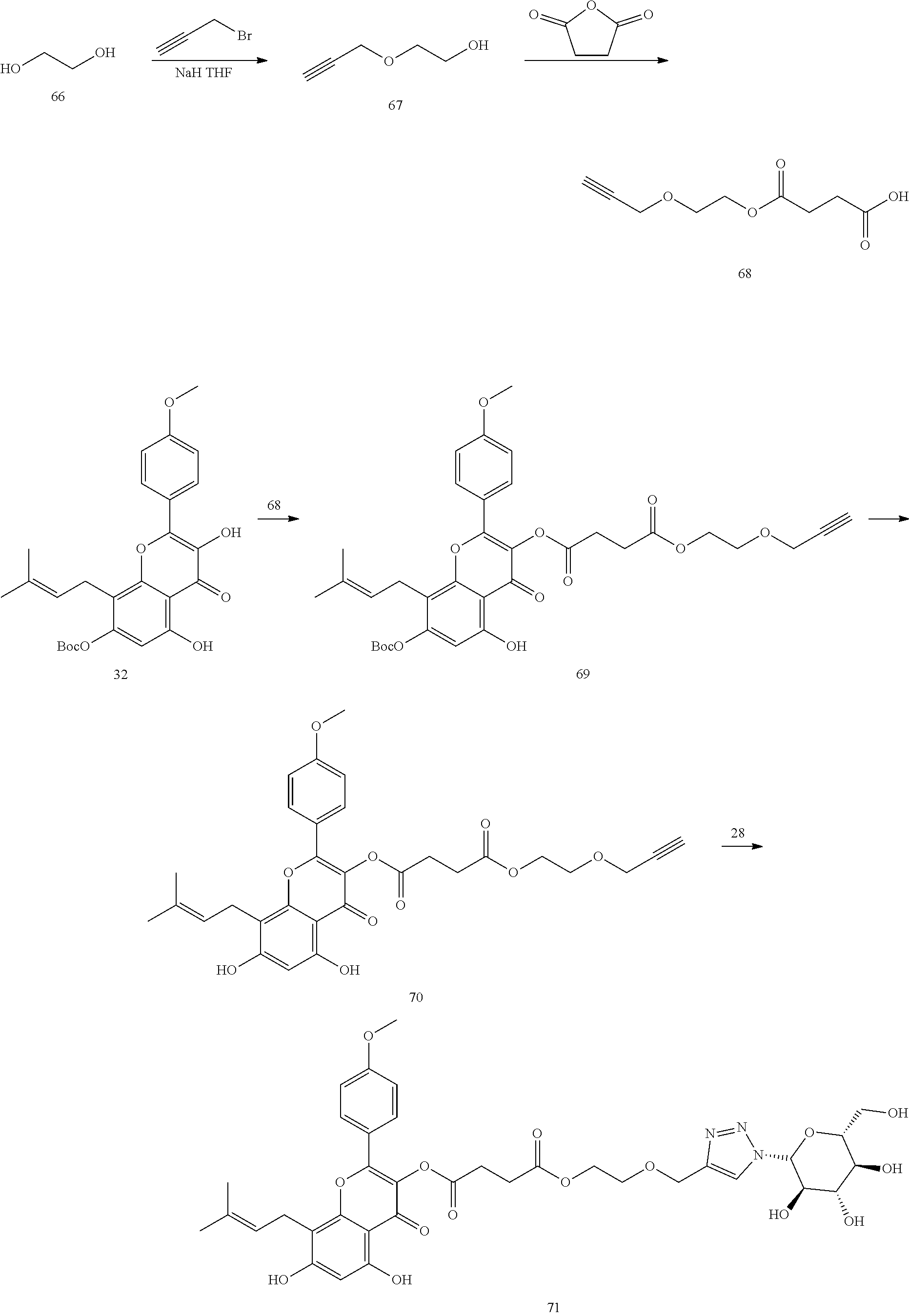

23 mL of glycol (compound 66) was dissolved in 100 mL of anhydrous tetrahydrofuran, the mixture was cooled to 0° C., and 5.5 g of sodium hydride was added into the mixture in small batches. The resulting mixture was subjected to a reaction at this temperature for 2 hours. 10 g of 3-bromopropyne was weighed and dissolved in 30 mL of anhydrous tetrahydrofuran, and then the obtained solution was dropwise added into the above reaction slowly. The resulting reactant was reacted at ambient temperature overnight. 20 mL of water was added to the resulting reactant. The resulting reactant was concentrated to remove tetrahydrofuran, and extracted with ethyl acetate (40 mL×3) to collect an organic layer. The organic layer was combined, dried over anhydrous sodium sulfate, and filtered. The obtained filtrate was concentrated and purified by column chromatography to obtain 5.5 g of compound 67, with a yield of 64.9%.

1 g of the compound 67 was dissolved in 20 mL of anhydrous dichloromethane, and 1.5 g of succinic anhydride and 122 g of DMAP were added thereto. The resulting mixture was stirred at 36° C. overnight, concentrated and purified by a column (DCM:MeOH=100:1-50:1) to obtain 1.1 g of compound 68 as a light yellow liquid, with a yield of 55.0%.

400 mg the compound 32 and 171 mg of the compound 68 were weighed and dissolved in 10 mL of dichloromethane, and 212 mg of EDCI and 36.5 mg of DMAP were added successively thereto. The resulting mixture was stirred at ambient temperature overnight, concentrated at reduced pressure in vacuum, and purified by a preparation plate (PE:EA=2:1) to obtain 200 mg of compound 69 as yellow oil, with a yield of 36.0%.

200 mg of the compound 69 was dissolved in 10 mL of dichloromethane, 2 mL of trifluoroacetic acid was added thereto. The mixture was stirred at ambient temperature overnight, concentrated and purified by a preparation plate (PE:EA=2:1) to obtain 100 mg of compound 70 as yellow oil, with a yield of 59.1%.

100 mg of the compound 70 and 38 mg of the compound 28 were dissolved in 8 mL of THF-H₂O mixed solvent (1:1), and 29 mg of anhydrous cupric sulfate and 36 mg of sodium ascorbate was added successively thereto. The resulting mixture was stirred at ambient temperature overnight, concentrated and purified by a flash column(DCM:MeOH=8:1) to obtain a crude product. The crude product was purified by a preparation plate (DCM:MeOH=8:1) to obtain 50 mg of compound 71 as a light yellow solid, with a yield of 36.5%.

¹H NMR (400 Hz, CD₃OD) δ 8.15 (s, 1H), 7.91 (d, J=8.8 Hz, 2H), 7.10 (d, K=9.2 Hz, 2H), 6.18 (s, 1H), 5.57 (d, J=9.2 Hz, 1H). 4.63 (s, 2H), 4.22-4.24 (m, 2H), 3.84-3.88 (m, 5H), 3.69-3.71 (m, 2H), 3.49-3.54 (m, 3H), 2.98 (t, J=6.6 Hz, 2H), 2.90 (t, J=6.6 Hz, 2H), 2.74 (t, J=6.6 Hz, 2H), 1.91 (t, J=6.6 Hz, 2H), 1.38 (s, 6H).

### EXAMPLE 11

### Synthesis of Compound 72

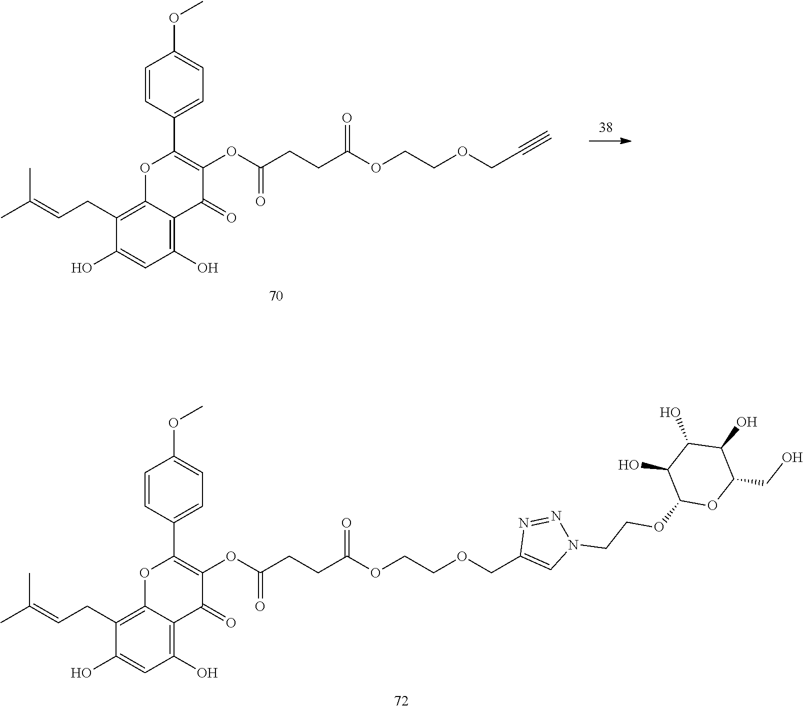

100 mg the compound 70 and 45 mg of the compound 38 were dissolved in 8 mL of THF-H₂O mixed solvent (1:1), and 29 mg of anhydrous cupric sulfate and 36 mg of sodium ascorbate were successively added thereto. The resulting mixture was stirred at ambient temperature overnight, concentrated and purified by a flash column (DCM:MeOH=8:1) to obtain a crude product. The crude product was purified by a preparation plate (DCM:MeOH=8:1) to obtain 18 mg of compound 72 as a light yellow solid, with a yield of 12.4%.

¹H NMR (400 Hz, CD₃OD) δ 8.05 (s, 1H), 7.86 (d, J=9.2 Hz, 2H), 7.04 (d, J=8.8 Hz, 2H), 6.14 (s, 1H), 4.57-4.59 (m, 4H), 4.27 (d, J=8.0 Hz, 1H), 4.18-4.24 (m, 3H), 3.95-3.98 (m, 1H), 3.85 (s, 3H), 3.82-3.84 (m, 3H), 3.61-3.70 (m, 3H), 124-3.26 (m, 2H), 3.14-3.18 (m, 1H), 2.95-2.98 (m, 2H), 2.84 J=6.6 Hz, 2H), 2.72-2.75 (m, 2H), 1.88 (t, J=6.8 Hz, 2H), 1.36 (s, 6H).

### EXAMPLE 12

### Synthesis of Compound 73

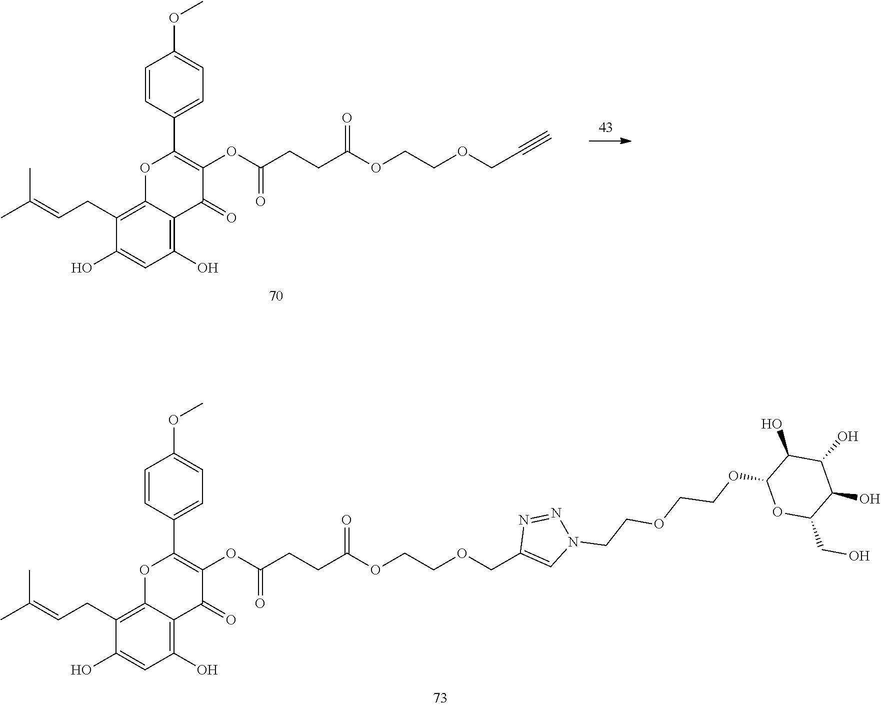

40 mg of the compound 70 and 21 tug of the compound 43 were dissolved in 8 mL of THF-H₂O mixed solvent (1:1), and 12 mg of anhydrous cupric sulfate and 14 mg of sodium ascorbate were added successively thereto. The resulting mixture was stirred at ambient temperature overnight, concentrated and purified by a flash column (DCM:MeOH=8:1) to obtain a crude product. The crude product was purified by a preparation plate (DCM:MeOH=8:1) to obtain 25 mg of compound 73 as a light yellow solid, with a yield of 40.9%.

¹H NMR (400 Hz, CD₃OD) 8.01 (s, 1H), 7.83 (d, J=8.8 Hz, 2H), 7.01 (d, J=8.8 Hz, 2H), 6.12 (s. 1H), 4.60 (s, 2H), 4.50-4.52 (m, 2H), 4.26 (d. J=7.6 Hz, 1H), 4.22-4.24 (m, 2H), 3.92-3.96 (m, 1H), 3.83-3.86 (m, 6H), 3.61-3.66 (m, 6H), 3.31-3.35 (m, 1H), 3.25-3.28 (m, 1H), 3.15-3.20 (m, 1H), 2.94-2.97 (m, 2H), 2.80 (t, J=6.8 Hz, 2H), 2.72-2.75 (m, 2H). 1.86 (t, J=6.6 Hz, 2H), 1.35 (s, 6H).

### EXAMPLE 13

### Synthesis of Compound 81

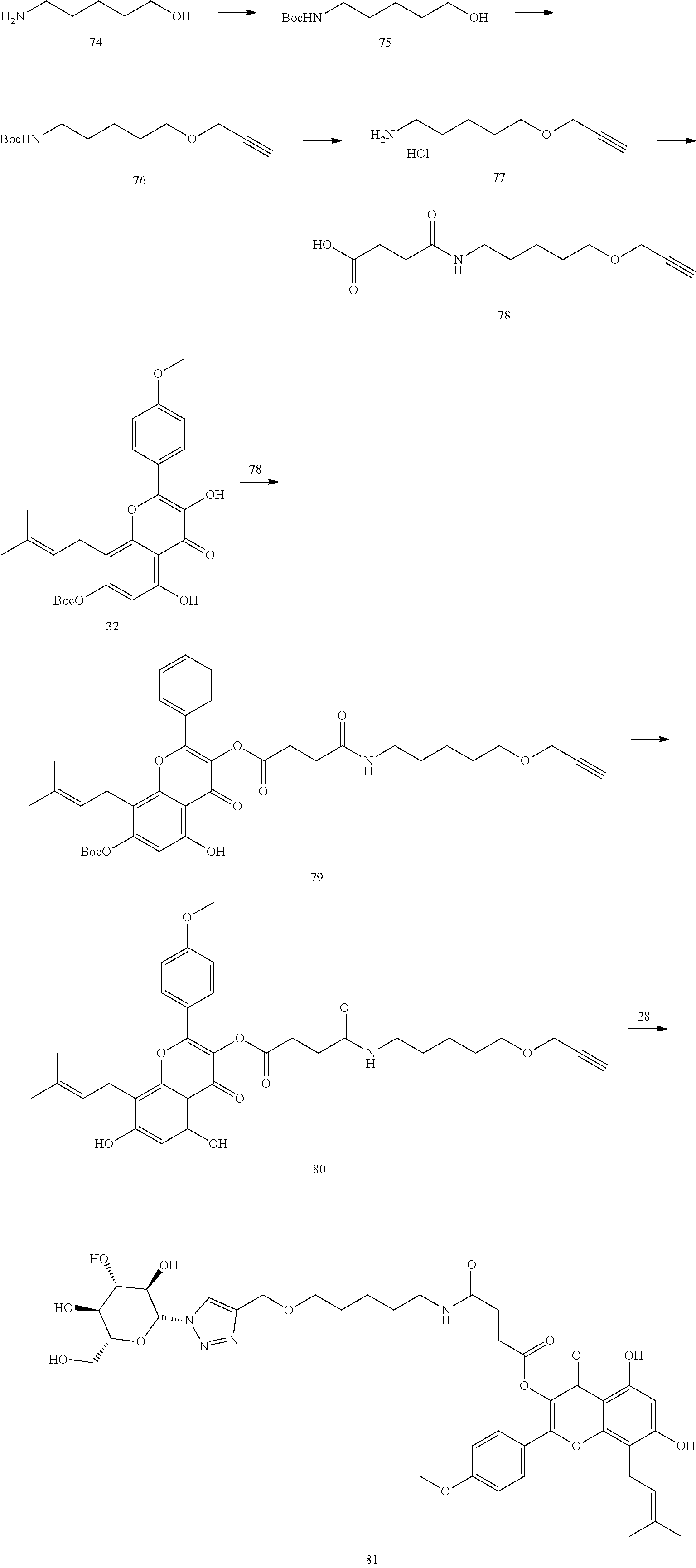

2.09 g of compound 74 and 5.6 mL of triethylamine were weighed and dissolved in 50 mL of dichloromethane, and the mixture was cooled to 0° C. Then 4.43 g of di-tert-but 1 Bicarbonate was dropwise added into the mixture slowly. The obtained mixture was subjected to a reaction at ambient temperature overnight. The resulting reactant was washed with 1N HCl (100 mL×3) to collect an organic layer. The organic layer was dried over anhydrous sodium sulfate, and filtered. The obtained filtrate was concentrated to obtain 4 of compound 75, with a yield of 97.3%.

4 g of the compound 75 was weighed and dissolved in 60 mL of tetrahydrofuran, and 812 mg of tetrabutylammonium iodide, 660 mg of sodium iodide and 3.92 g of 3-bromopropyne were added thereto. Then 2.48 g of potassium hydroxide was added into the mixture in small batches while stirring at ambient temperature. The resulting mixture was subjected to a reaction at ambient temperature overnight. The resulting reactant was concentrated to remove tetrahydrofuran, and 30 mL of water was added thereto. The resulting reactant /as extracted with ethyl acetate (30 mL×3) to collect an organic layer. The organic layer was dried over anhydrous sodium sulfate, and filtered. The obtained filtrate was concentrated and purified by column chromatography to obtain 2.8 g of compound 76, with a yield of 59.1%.

2.8 g of the compound 76 was weighed and dissolved in 40 mL of methanol, and 40 mL of 3N HCl solution was added thereto. The mixture was stirred at ambient temperature overnight, and concentrated to obtain 1.7 g of compound 77, with a yield of 86.7%.

1.5 g of the compound 77 was weighed and suspended in 30 mL of dichloromethane, and 2.6 mL of triethylamine, 1.2 g of succinic anhydride and 224 mg of DMAP were added successively thereto. The resulting mixture was subjected to a reaction at 40° C. for 15 hours. Then the resulting reactant was cooled, washed with 1N HCl (30 mL×2) and a saturated sodium chloride solution (once) successively, dried over anhydrous sodium sulfate, and filtered. The obtained filtrate was concentrated to obtain 200 mg of compound 78 as a yellow solid, with a yield of 9.6%.

255 mg of the compound 32 and 125 mg of the compound 78 were weighed and dissolved in 10 mL dichloromethane, and 135 mg of EDCI and 25 mg of DMAP were added successively thereto. The mixture was stirred at ambient temperature overnight, concentrated at reduced pressure in vacuum, and purified by a preparation plate (DCM:MeOH=10:1) to obtain 150 mg of compound 79 as yellow oil, with a yield of 41.6%.

150 mg of the compound 79 was dissolved in 10 mL of dichloromethane, and 4 mL of trifluoroacetic acid was added thereto. The mixture was stirred at ambient temperature overnight, concentrated and purified by a preparation plate (DCM:MeOH=15:1) to obtain 60 mg of compound 80 as yellow oil, with a yield of 47.2%.

60 mg of the compound 80 and 21 mg of the compound 28 were dissolved in 8 mL of THF-H₂O mixed solvent (1:1), and 16 mg of anhydrous cupric sulfate and 20 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH=8:1) to obtain 25 mg of compound 81 as a light yellow solid, with a yield of 29.4%.

¹H NMR (400 Hz, CD₂OD) δ 8.12 (s, 1H), 7.91 (d. J=9.2 Hz, 2H), 7.11 (d, J=9.2 Hz, 2H), 6.18 (s, 1H), 5.58 (d, J=9.2 Hz, 1H), 4.53 (s, 2H), 3.85-3.90 (m, 5H), 3.68-3.72 (m, 1H), 3.44-3.56 (m, 5H), 3.15 (t, J=6.8 Hz, 2H), 2.98 (t, J=6.8 Hz, 2H), 2.89 (t, J=6.8 Hz, 2H), 2.58 (t, J=6.8 Hz, 2H), 1.91 (t, J=6.6 Hz, 2H), 1.43-1.57 (m, 4H), 1.37 (s, 6H), 1.28-1.34 (m, 2H).

### EXAMPLE 14

### Synthesis of Compound 87

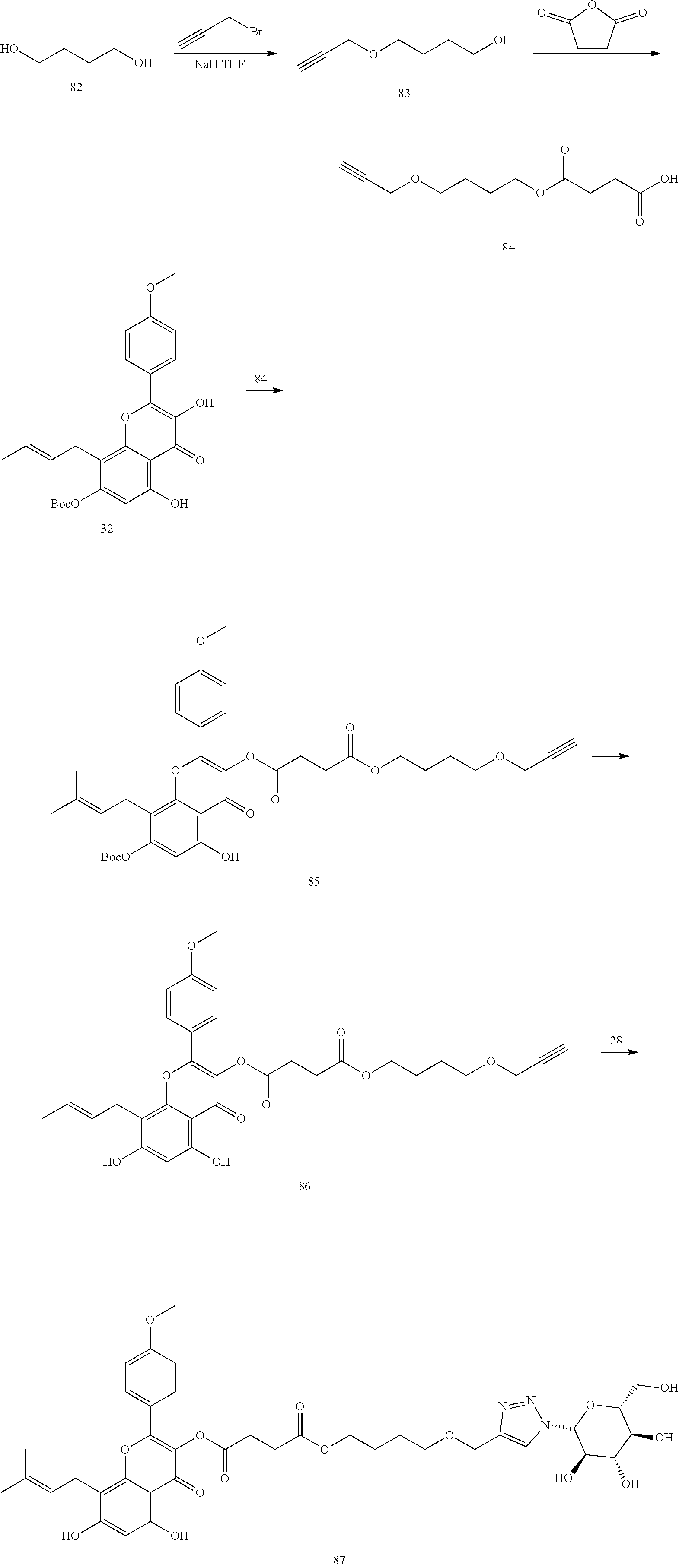

19.1 g of compound 82 was dissolved in 100 mL of anhydrous tetrahydrofuran., and the mixture was cooled to 0° C. Then 3.4 g of sodium hydride was added into the mixture in small batches. The resulting mixture was subjected to a reaction at this temperature for 2 hours. 5 g of 3-bromopropyne was weighed and dissolved in 30 mL of anhydrous tetrahydrofuran, and then the obtained solution was dropwise added into the above reaction slowly. The resulting reactant was reacted at ambient temperature overnight. 20 n L of water was added to the resulting reactant. The resulting reactant was concentrated to remove tetrahydrofuran, and extracted with ethyl acetate (40 mL×3) to collect an organic layer. The organic layer was combined, dried over anhydrous sodium sulfate, and filtered. The obtained filtrate was concentrated and purified by column chromatography to obtain 5.0 g of compound 83, with a yield of 92.6%.

3.6 g of the compound 83 was dissolved in 30 mL of anhydrous dichloromethane, and 4.2 g of succinic anhydride and 343 mg of DMAP were added thereto. The mixture was stirred at 36° C. overnight, concentrated and purified by a column (PE:EA=30:1-2:1) to obtain 2.4 g of compound 84 as a light yellow liquid, with a yield of 37.4%.

367 mg of the compound 32 and 180 mg of the compound 84 were weighed and dissolved in 10 mL of dichloromethane, and 199 mg of EDCI and 35 mg of DMAP were added successively thereto. The mixture was stirred at ambient temperature overnight, concentrated at reduced pressure in vacuum, and purified by a preparation plate (PE:EA=4:1) to obtain 200 mg of compound 85 as yellow oil, with a yield of 37.6%.

200 mg of the compound 85 was dissolved in 10 mL of dichloromethane, and 3 mL of trifluoroacetic acid was added thereto. The mixture was stirred at ambient temperature overnight, concentrated and purified by a preparation plate (PE:EA=3:1) to obtain 100 mg of compound 86 as a yellow solid, with a yield of 58.8%.

30 mg of the compound 86 and 30 mg of the compound 28 were dissolved in 8 mL of THF-H₂O mixed solvent (1:1), and 30 mg of anhydrous cupric sulfate and 50 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH==8:1) to obtain 25 mg of compound 87 as a light yellow solid, with a yield of 60.9%.

¹H NMR (400 Hz, CD₃OD) δ 8.14 (s, 1H), 7.91 (d, J=9.2 Hz, 2H), 7.09 (d, J=8.8 Hz, 2H), 6.18 (s, 1H), 5.58 (d, J=9.2 Hz, 1H), 4.57 (s, 2H), 4.08 (t, J=6.2 Hz, 2H), 3.85-3.90 (m, 5H), 3.68-3.72 (m, 1H), 3.53-3.55 (m, 1H), 3.48-3.52 (m, 3H), 2.96-2.99 (m, 2H), 2.89 (t, J=7.0 Hz, 2H), 2.70-2.73 (m, 2H), 1.91 (1 J=6.8 Hz, 2H), 1.58-1.62 (m, 4H), 1.38 (s, 6H).

### EXAMPLE 15

### Synthesis of Compound 88

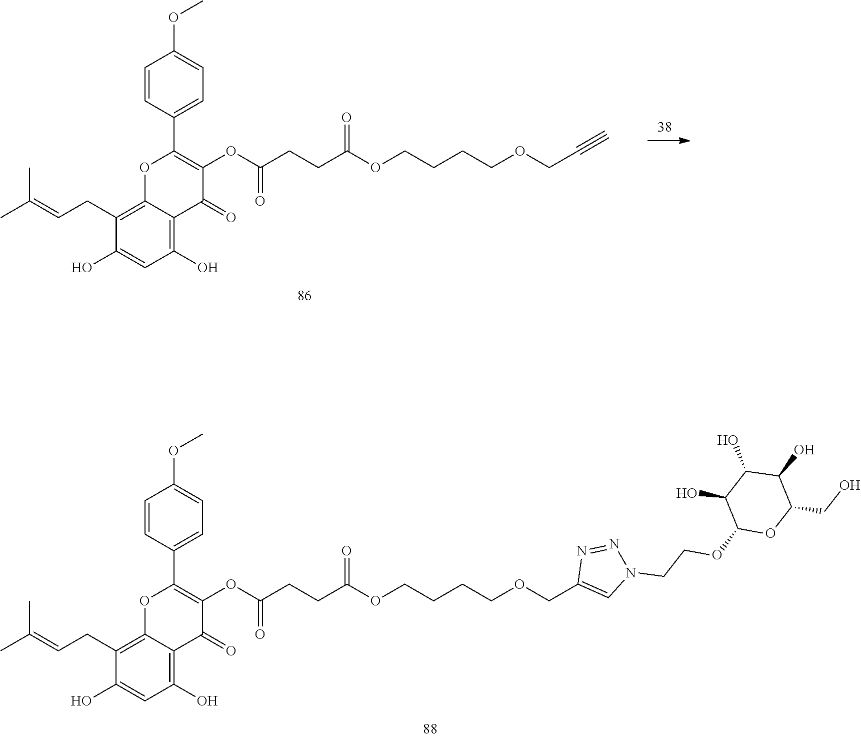

40 mg of the compound 86 and 40 mg of the co pound 38 were dissolved in 8 of THF-H₂O mixed solvent (1:1), and 40 mg of anhydrous cupric sulfate and 60 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH=8:1) to obtain 25 mg of compound 88 as a light yellow solid, with a yield of 43.9%.

¹H NMR (400 Hz, CD₃OD) δ 8.04 (s, 1H), 7.92 (d, J=9.2 Hz, 2H), 7.11 (d, J=9.2 Hz, 2H), 6.19 (s, 1H), 4.61 (t, J=4.2 Hz, 2H), 4.53 (s, 2H), 4.29 (d, J=8.0 Hz, 1H), 4.19-4.24 (m, 1H), 4.08 (t, J=6.2 Hz, 2H), 3.96-4.01 (m, 1H), 3.89 (s, 3H), 3.83-3.86 (m, 1H), 3.61-3.66 (m, 1H), 3.49 (t, J=6.0 Hz, 2H), 3.24-3.26 (m, 2H), 3.14-3.18 (m, 1H), 2.96-3.00 (m, 2H), 2.90 (t, J=6.8 Hz, 2H), 2.70-2.73 (m, 2H), 1.92 (t, J=7.0 Hz, 2H), 1.58-1.63 (m, 4H), 1.38 (s, 6H).

### EXAMPLE 16

### Synthesis of Compound 89

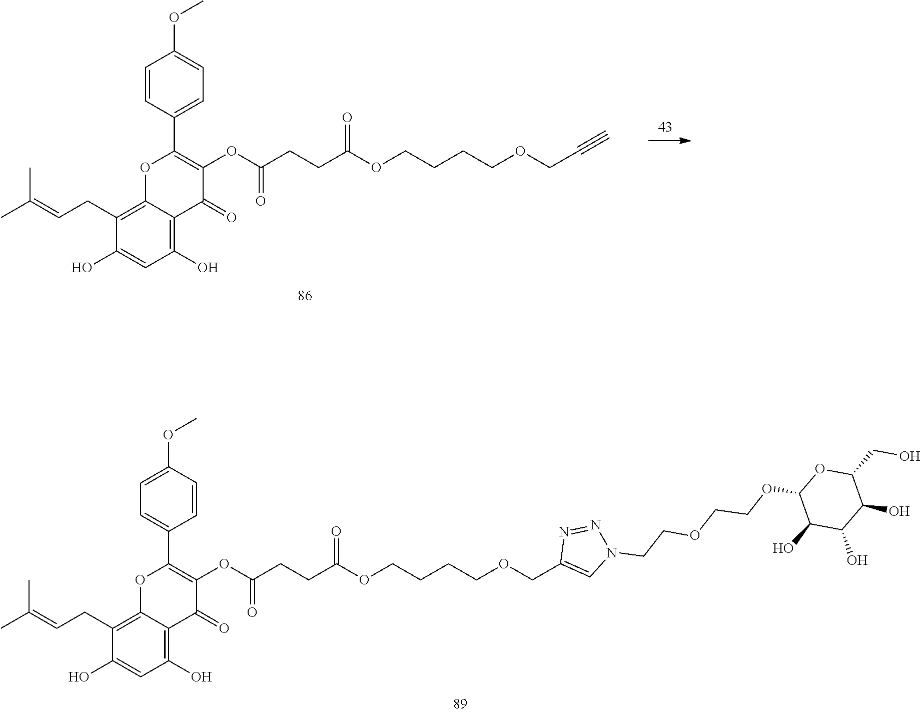

30 mg of the compound 86 and 30 mg of the compound 43 were dissolved in 8 mL THF-H₂O mixed solvent (1:1), and 30 tug of anhydrous cupric sulfate and 50 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH=8:1) to obtain 25 mg of compound 89 as a light yellow solid, with a yield of 56.8%.

¹H NMR (400 Hz, CD₃OD) δ 8.00 (s, 1H), 7.89 (d, J=8.8 Hz, 2H), 7.07 (d, J=8.8 Hz, 2H), 6.16 (s, 1H), 4.54 (t, J=5.0 Hz, 4H), 4.27 (d, J=8.0 Hz, 1H), 4.07 4, J=6.2 Hz, 2H), 3.92-3.96 (m, 1H), 3.83-3.89 (m, 6H), 3.62-3.70 (m, 4H), 3.49 (t, J=5.8 Hz, 2H), 3.34-3.36 (m, 1H), 3.24-3.26 (m, 1H), 3.15-3.19 (m, 1H), 2.95-2.99 (m, 2H), 2.87 (t, J=6.6 Hz, 2H), 2.70-2.73 (m, 2H), 1.90 (t, J=6.6 Hz, 2H), 1.59-1.64 (m, 4H), 1.37 (s, 6H).

### EXAMPLE 17

Synthesis of Compound 95

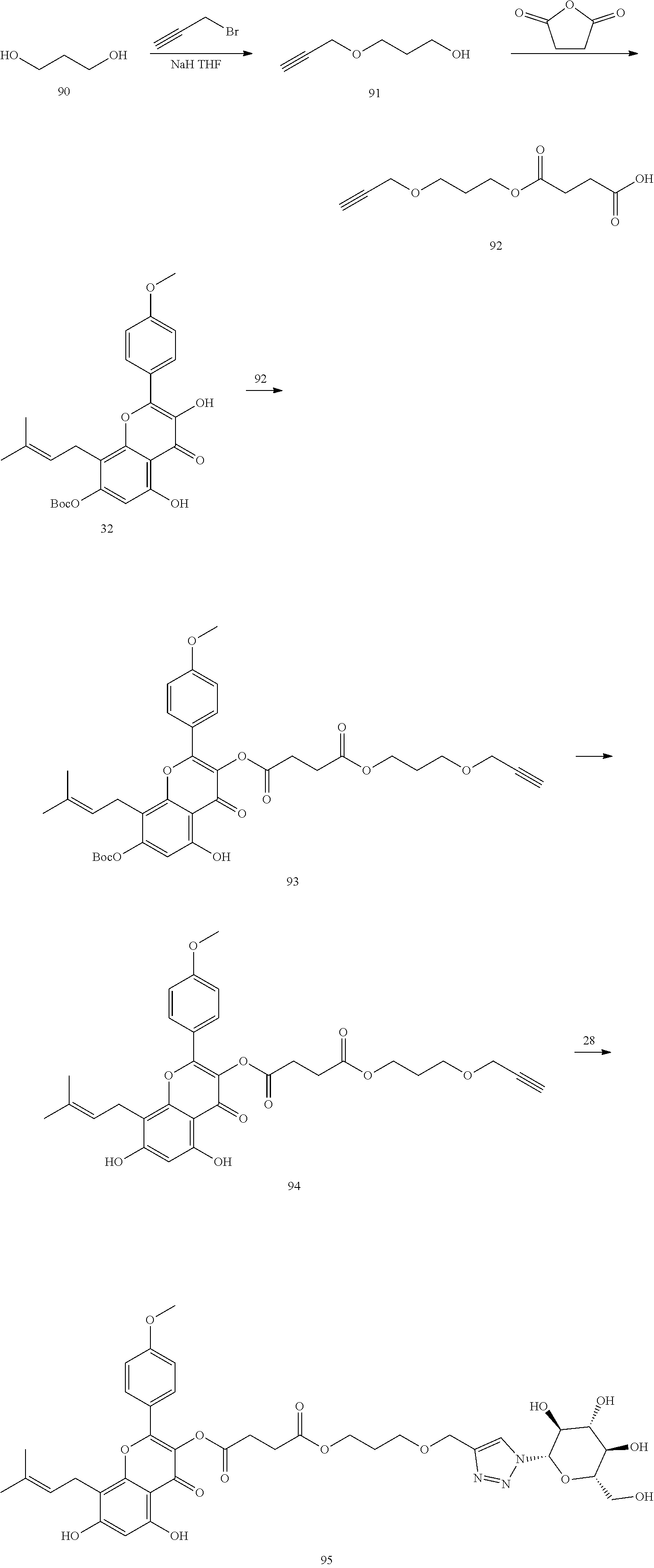

16.1 g of compound 90 was dissolved in 150 mL of anhydrous THF, and 3.4 g

of sodium hydride was added into the mixture slowly in an ice bath. The resulting mixture was subjected to a reaction at low temperature for 3 hours. 5 g of propargyl bromide was dissolved in 30 mL of THF, and then the obtained solution was dropwise added into the resulting reactant. The resulting reactant was reacted at ambient temperature overnight, and water was added thereto. The resulting reactant was concentrated to remove THF, extracted with ethyl acetate twice, and purified by a column (eluted with PE:DCM (3:1), DCM, and DCM:MeOH (50:1) in sequence) to obtain 1.15 g of compound 91.

1.15 g of the compound 91 was dissolved in 20 mL of anhydrous dichloromethane, and 2 g of succinic anhydride and 123 mg of DMAP were added thereto. The mixture was stirred at 36° C. overnight, concentrated and purified by a column (PE:EA=30:1-2:1) to obtain 500 mg of compound 92 as a light yellow liquid.

344 mg of the compound 32 and 156 mg of the compound 92 were weighed and dissolved in 10 mL of dichloromethane, and 181 mg of EDCI and 32 mg of DMAP were added successively thereto. The mixture was stirred at ambient temperature overnight, concentrated at reduced pressure in vacuum, and purified by a preparation plate PE:EA=5:1) to obtain 180 mg of compound 93 as yellow oil.

180 mg of the compound 93 was dissolved in 10 mL of dichloromethane, and 3 mL of trifluoroacetic acid was added thereto. The mixture was stirred at ambient temperature overnight, concentrated and purified by a preparation plate (PE:EA=3:1) to obtain 100 mg of compound 94 as a yellow solid.

30 mg of the compound 94 and 30 mg of the compound 28 were dissolved in 8 mL of THF-H₂O mixed solvent (1:1), and 30 mg of anhydrous cupric sulfate and 50 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH —8:1) to obtain 25 mg of compound 95 as a light yellow solid.

EXAMPLE 18 Synthesis of Compound 96

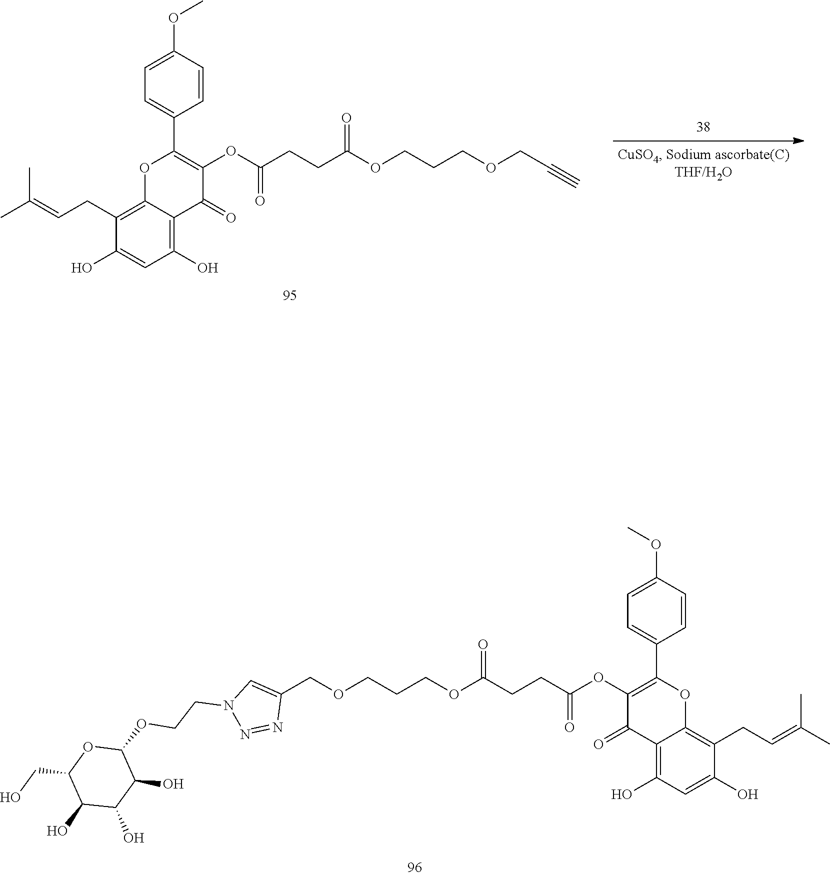 [SMILES: C#CCOCCCOC(=O)CCC(=O)Oc1c(-c2ccc(OC)cc2)oc2c(CC=C(C)C)c(O)cc(O)c2c1=O ; COc1ccc(-c2oc3c(CC=C(C)C)c(O)cc(O)c3c(=O)c2OC(=O)CCC(=O)OCCCOCc2cn(CCO[C@H]3O[C@@H](CO)[C@H](O)[C@@H](O)[C@@H]3O)nn2)cc1]

30 mg of the compound 95 and 30 mg of the compound 38 were dissolved in 8 mL of THF-H₂O mixed solvent (1:1), and 30 mg of anhydrous cupric sulfate and 50 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH=8:1) to obtain 25 mg of compound 96 as a light yellow solid.

### EXAMPLE 19

### Synthesis of Compound 97

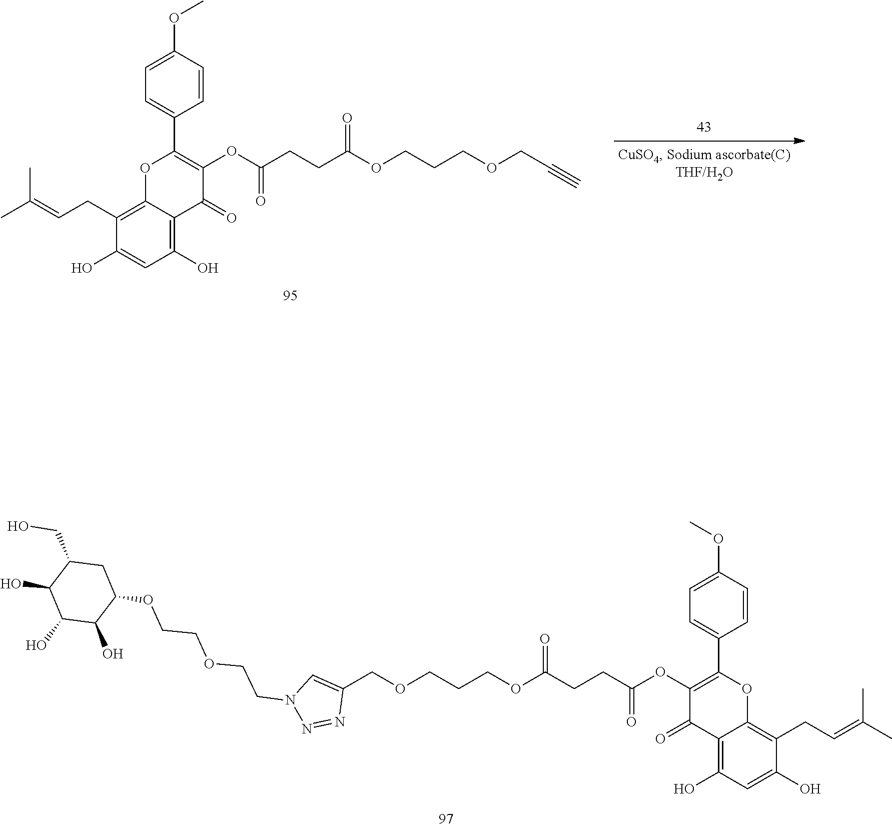 [SMILES: C#CCOCCCOC(=O)CCC(=O)Oc1c(-c2ccc(OC)cc2)oc2c(CC=C(C)C)c(O)cc(O)c2c1=O ; COc1ccc(-c2oc3c(CC=C(C)C)c(O)cc(O)c3c(=O)c2OC(=O)CCC(=O)OCCCOCc2cn(CCOCCO[C@H]3C[C@@H](CO)[C@H](O)[C@@H](O)[C@@H]3O)nn2)cc1]

30 mg of the compound 95 and 30 mg of the compound 43 were dissolved in 8 mL, of THF-H₂O mixed solvent (1:1), and 30 mg of anhydrous cupric sulfate and 50 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH=8:1) to obtain 25 mg of compound 97 as a light yellow solid.

### EXAMPLE 20

### Synthesis of Compound 103

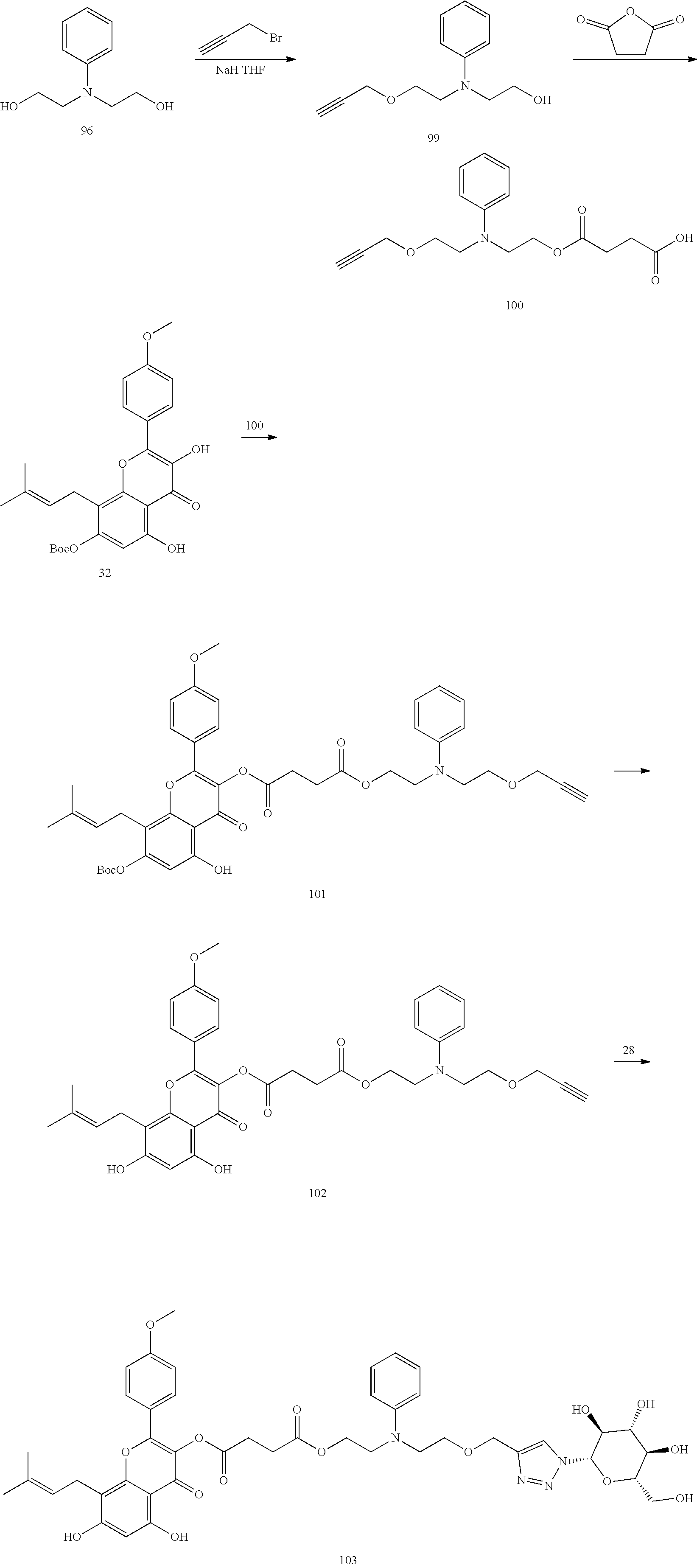

38 g of compound 98 was dissolved in 150 mL of anhydrous THF, and 3.4 g of sodium hydride was added into the mixture slowly in a ice bath. The mixture was subjected to a reaction in the ice bath for 3 hours. 5 g of propargyl bromide was dissolved in 30 mL, of THF, and the obtained solution was dropwise added into the above reaction. The resulting reactant was reacted at ambient temperature overnight. Then water was added into the resulting reactant. The resulting reactant was concentrated to remove THF, extracted with ethyl acetate twice, and purified by a column (eluted with PE:DCM (3:1), DCM, and DCM:MeOH (50:1) in sequence) obtain 11 g of compound 99.

2.1 g of the compound 99 was dissolved in 20 mL of anhydrous dichloromethane, and 1.44 g of succinic anhydride and 117 mg of DMAP was added thereto. The mixture was stirred at 36° C. overnight, concentrated and purified by a column (PE:EA=30:1-2:1) to obtain 2.9 g of compound 100 as a light yellow liquid.

344 mg of the compound 32 and 200 mg of the compound 100 were weighed and dissolved in 10 mL of dichloromethane, and 181 mg of EDCI and 32 mg of DMAP were added successively thereto. The mixture was stirred at ambient temperature overnight, concentrated at reduced pressure in vacuum, and purified by a preparation plate (PE:EA=5:1) to obtain 237 mg of compound 101 as yellow oil.

237 mg of the compound 101 was dissolved in 10 mL of dichloromethane, and 3 mL of trifluoroacetic acid was added thereto. The mixture was stirred at ambient temperature overnight, concentrated and purified by a preparation plate (PE:EA=3:1) to obtain 168 mg of compound 102 as a yellow solid.

30 mg of the compound 102 and 30 mg of the compound 28 were dissolved in 8 mL of THF-H₂O mixed solvent (1:1), and 30 mg of anhydrous cupric sulfate and 50 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH=8:1) to obtain 25 mg of compound 103 as a light yellow solid.

¹H NMR (400 MHz, CD₃OD) δ 8.04 (s, 1H), 7.88 (d, J=8.9 Hz, 2H), 7.14 (dd, J=8.6, 7.4 Hz, 2H), 7.06 (d, J=8.9 Hz, 2H), 6.74 (d, J=8.2 Hz, 2H), 6.62 (s, 1H), 6.16 (s, 1H), 5.55 (d, J=9.2 Hz, 1H). 4.58 (s, 2H), 4.22 (t. =6.2 Hz, 2H), 3.90-3.81 (m, 5H), 3.66 (dd, J=16.7, 10.8 Hz, 3H), 3.59-3.50 (m, 6H), 2.96-2.90 (m, 2H), 2.86 (t, J=6.7 Hz, 2H), 2.72-2.62 (m, 2H), 1.89 (t, J=6.7 Hz, 2H), 1.37 (s, 6H).

EXAMPLE 21 Synthesis of Compound 104

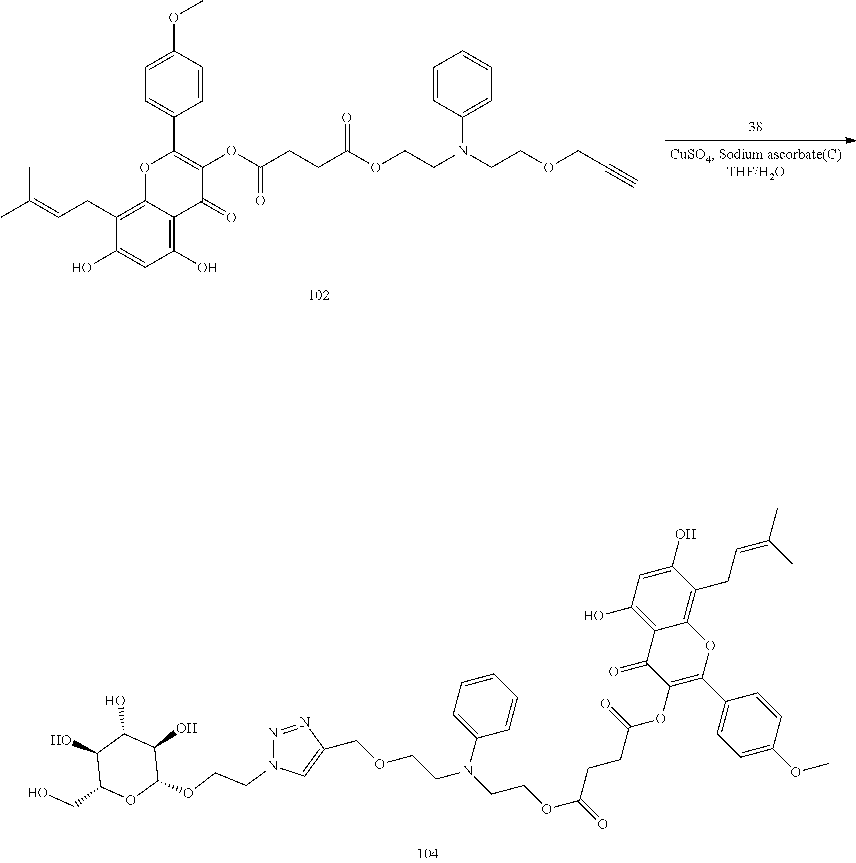 [SMILES: C#CCOCCN(CCOC(=O)CCC(=O)Oc1c(-c2ccc(OC)cc2)oc2c(CC=C(C)C)c(O)cc(O)c2c1=O)c1ccccc1 ; COc1ccc(-c2oc3c(CC=C(C)C)c(O)cc(O)c3c(=O)c2OC(=O)CCC(=O)OCCN(CCOCc2cn(CCO[C@@H]3O[C@H](CO)[C@@H](O)[C@H](O)[C@H]3O)nn2)c2ccccc2)cc1]

30 mg of the compound 102 and 30 mg of the compound 38 were dissolved in 8 mL of THF-H₂O mixed solvent (1:1), and 30 tug of anhydrous cupric sulfate and 50 mg of sodium ascorbate were added successively thereto. The mixture was stirred at ambient temperature overnight, concentrated and purified by a flash column (DCM:MeOH=8:1) to obtain a crude product. The crude product was purified by a preparation plate (DCM:MeOH =8:1) to obtain 20 mg of compound 104 as a light yellow solid.

¹ H NMR (400 MHz, CD₃OD) 7,97 (s, 1H), 7.89 (d, J=9.0 Hz, 2H), 7.10 (dd, J=18.2,5.2 Hz, 4H), 6.70 (d, J=8.1 Hz, 2H), 6.59 (t, J=7.3 Hz, 1H), 6.18 (s, 1H), 4.55 (d, J=5.6 Hz, 4H), 4.27 (d, J=7.8 Hz, 1H), 4.20 (d, J=6.4 Hz, 3H), 3.99-3.91 (m, 1H), 3.84 (s, 4H), 3.63 (d, J=5.4 Hz, 3H), 3.55 (d, J=14.1 Hz, 4H), 3.26 (s, 2H), 3.15 (dd, J=9.0, 7.8 Hz, 1H), 2.98-2.92 (m, 2H), 2.88 (s, 2H), 2.73-2.65 (m, 2H), 1.91 (s, 2H), 1.38 (s, 6H).

EXAMPLE 22

### Synthesis of Compound 105

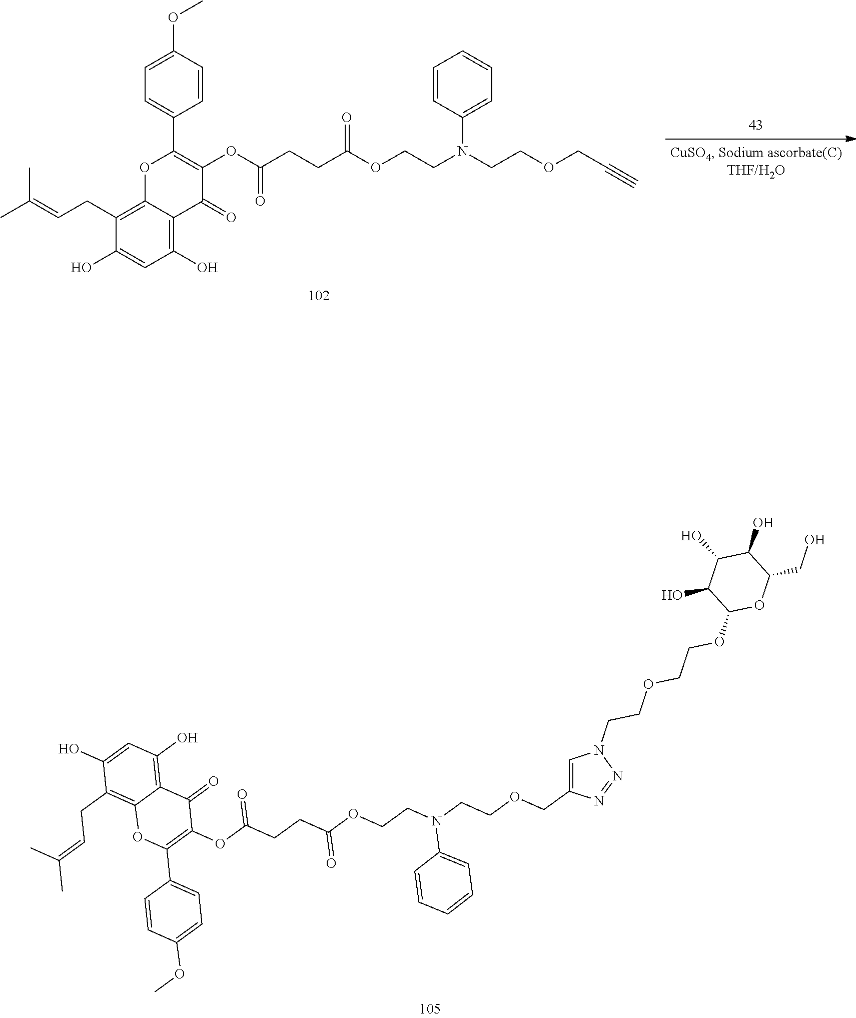

30 mg of the compound 102 and 30 mg of the compound 43 were dissolved in 8 mL THF-H₂O mixed solvent (1:1), and 30 mg of anhydrous cupric sulfate and 50 mg of sodium ascorbate were added successively thereto. The mixture was stirred at ambient temperature overnight, concentrated and purified by a flash column (DCM:MeOH=8:1.) to obtain a crude product. The crude product was purified by a preparation plate (DCM:MeOH=8:1) to obtain 20 mg of compound 105 as a light yellow solid.

¹H NMR (400 MHz, CD₃OD) δ 7.95-7.87 (m, 3H), 7.16-7.04 (m, 4H), 6.70 (d, J=8.1. Hz, 2H), 6.59 (s, 1H), 6.18 (s, 1H), 4.55 (s, 2H), 4.51-4.45 (m, 2H), 4.25 (d, J=7.8 Hz, 1H), 4.21 (t, J=6.3 Hz, 2H), 3.97-3.90 (m, 1H), 3.89-3.79 (m, 6H), 3.69-3.51 (m, 1.0H), 3.26 (d, J=8.2 Hz, 2H), 3.19-3.14 (m, 1H), 2.98-2.92 (m, 2H), 2.89 (t, J=6.8 Hz, 2H), 2.73-2.65 (m, 2H), 1.91 (t, J=6.8 Hz, 2H), 1.38 (s, 6H).

The data of 1 H NMR spectrum documented at the end of each example above are the data of the spectrum of the final compound prepared in the corresponding examples.

### EXAMPLE 23

### Determination of Solubility

About 1.325 mg of Icaritin and the synthesized derivative were weighed and dissolved in 1.5 mL methanol respectively, then diluted with methanol into solutions of 10, 20, 50, 100, 150, 200, 500 μg/mL, and filtered over an organic-system filter head of 0.45 μm into sample bottles, obtaining the samples for test. Four parts of Icaritin and the synthesized derivative were weighed respectively, 1.00 mg for each part, and placed into 1.5 mL EP tubes, and 1 mL of water, normal saline, PBS, and 0.1% Tween 80 were respectively added thereto. The obtained solutions were ultrasonicated for 1 h, placed in a water bath at 37° C. overnight, and centrifuged at 10000 r/min for 3 min to collect supernatants. The supernatants were taken into sample bottles and detected by HPLC. The experiments were conducted in parallel for three times. The detection of HPLC is conducted under conditions of a chromatographic column of waters Symmetry C18, a mobile phase of a solution of methanol and water with a ratio of 80:20, a flow rate of 1.0 mL/min, a column temperature of 30° C., a detection wavelength of 210 nm, a run time of 30 min, and a sample load of 20 μL. The chromatogram was recorded, and the result is shown in Table 1.

TABLE 1
Determination on the Solubility of Icaritin and its derivatives (x̅ + s, n = 3)

| | | Solubility (μg/mL) | | | |
|---|---|---|---|---|---|
| Samples | Linear equation | Water | Normal Saline | PBS | 0.1% Tween 80 |
| Icaritin | y = 87601x − 707717 | 8.570 ± 0.832 | 8.104 ± 1.012 | 8.099 ± 0.897 | 8.111 ± 0.672 |
| | R² = 0.9981 | | | | |
| 35 | y = 82565x − 986504 | 11.985 ± 1.231 | 12.309 ± 1.312 | 11.979 ± 1.023 | 12.073 ± 1.031 |
| | R² = 0.9989 | | | | |
| 39 | y = 40044x + 162204 | 13.871 ± 1.031 | 13.828 ± 1.123 | 13.825 ± 0.937 | 13.819 ± 1.323 |
| | R² = 0.9973 | | | | |
| 44 | y = 53956x − 593213 | 11.292 ± 0.831 | 11.035 ± 1.029 | 11.037 ± 1.039 | 11.231 ± 1.092 |
| | R² = 0.9978 | | | | |
| 50 | y = 43812x − 197579 | 4.551 ± 0.324 | Insoluble | 4.534 ± 0.347 | 4.639 ± 1.274 |
| | R² = 0.9971 | | | | |
| 51 | y = 42960x − 228586 | 50.321 ± 1.313 | 47.948 ± 0.993 | 48.492 ± 0.948 | 51.508 ± 1.231 |
| | R² = 0.9961 | | | | |
| 57 | y = 56593x − 924669 | 17.710 ± 1.012 | 16.383 ± 0.941 | 16.391 ± 1.029 | 6.508 ± 0.742 |
| | R² = 0.9954 | | | | |
| 58 | y = 47835x − 173207 | 3.641 ± 0.374 | 3.681 ± 0.947 | 3.823 ± 0.874 | 3.889 ± 0.941 |
| | R² = 0.9998 | | | | |
| 64 | y = 48509x − 46233 | 2.241 ± 0.941 | Insoluble | 0.991 ± 0.314 | 1.083 ± 0.756 |
| | R² = 0.9998 | | | | |
| 65 | y = 43943x + 396561 | Insoluble | Insoluble | Insoluble | Insoluble |
| | R² = 0.9993 | | | | |
| 71 | y = 47496x − 266999 | Insoluble | Insoluble | Insoluble | Insoluble |
| | R² = 0.9991 | | | | |
| 72 | y = 47496x − 266999 | 5.852 ± 0.748 | 5.673 ± 1.031 | 5.656 ± 1.131 | 5.697 ± 1.312 |
| | R² = 0.9991 | | | | |
| 73 | y = 40044x + 162204 | Insoluble | Insoluble | Insoluble | Insoluble |
| | R² = 0.9973 | | | | |
| 81 | y = 42999x + 195037 | Insoluble | Insoluble | Insoluble | Insoluble |
| | R² = 0.9988 | | | | |
| 87 | y = 65175x − 67386 | 1.277 ± 0.836 | 1.386 ± 0.743 | Insoluble | 1.358 ± 0.235 |
| | R² = 0.9999 | | | | |
| 88 | y = 61794x − 454748 | 7.597 ± 1.038 | 7.399 ± 0.384 | Insoluble | 8.384 ± 1.363 |
| | R² = 0.9990 | | | | |
| 89 | y = 52896x + 83977 | Insoluble | Insoluble | Insoluble | Insoluble |
| | R² = 0.9998 | | | | |
| 95 | y = 64863x − 101030 | 1.741 ± 0.123 | 1.684 ± 0.234 | Insoluble | 1.598 ± 0.521 |
| | R² = 0.9997 | | | | |
| 96 | y = 72511x − 1E+06 | 16.871 ± 0.462 | 16.835 ± 0.313 | 15.492 ± 1.31 | 16.828 ± 0.422 |
| | R² = 0.9944 | | | | |
| 97 | y = 75069x − 375793 | 5.474 ± 0.394 | 5.036 ± 1.031 | 5.031 ± 0.901 | 6.123 ± 0.931 |
| | R² = 0.9964 | | | | |
| 103 | y = 81153x − 477449 | 6.025 ± 1.021 | 5,909 ± 0.314 | 5.957 ± 1.031 | 5.943 ± 1.123 |
| | R² = 0.9923 | | | | |
| 104 | y = 67826x + 494475 | Insoluble | Insoluble | Insoluble | Insoluble |
| | R² = 0.9981 | | | | |
| 105 | y = 85616x − 2E+06 | 23.610 ± 1.231 | 23.941 ± 0.982 | Insoluble | 23.439 ± 1.120 |
| | R² = 0.9982 | | | | |

It can be known from Table 1 that the solubility of compounds 35, 39, 44, 51, 57, 96, 105 is improved effectively compared with that of the hulk drug.

### EXAMPLE 24

### Anti-tumor Activity in Vitro

The effects of Icaritin and its derivatives on the activities of HepG2 and SMMC7721 hepatoma cells were investigated by a MTT method. The HepG2 and SMMC7721 cells were inoculated on a 96-well plates at a density of 2000/well, and cultivated for 24 h in an environment of 37° C. and 5% CO₂ so that the cells adhered to the wall fully. The cells were then treated by respectively administering 100 μL solution of Icaritin or the derivatives of Icaritin at different concentrations (which are cultivated by DMED containing 10% fetal calf serum and 1% penicillin-streptomycin and the drug stock solutions were diluted to: 0.78125, 1.5625, 3.125, 6.25, 12.5, 25, 50, 100 1.1M). 6 duplicate wells were set for each concentration, and only an equal volume of culture medium was added into the blank control group. After reaction for 48, 7 2 h, each well was added with 20 μL 5 mg/mL, of MTT solution, and incubated in a cell incubator for 3-4 h. Then the liquid in the well plates was sucked to discard. Each well was added with 150 μL of dimethyl sulfoxide (DMSO), and shaken on an enzyme-labeled instrument for 30 s at ambient temperature so that the Formazan crystals were dissolved sufficiently. The absorbance value A of each well at 570 nm was detected by the enzyme-labeled instrument, and the experiment was repeated for 3 times. The inhibition rates of Icaritin and its derivative on the growth of tumors cells were calculated according to the following formula and the median inhibitory concentration (IC₅₀ value) of the drug was calculated using Graph pad prism 5, and the results are shown in Table 2. No record of IC 50 value means that when the drug concentration is 100 μM, its inhibition rate on cells is less than 50%, and the IC₅₀ value cannot be calculated.

inhibition rate on tumor cells %=1-[(OD Average of Experimental group-OD Average of Blank control group)/(OD Average of cell control group-OD Average of Blank control group)]×100%.

TABLE 2
Effects of Icaritin and its derivatives on
the activity of HepG2 and SMMC721 cells

| | IC₅₀ (μM) | | | |
|---|---|---|---|---|
| | HepG2 | | SMMC7721 | |
| Samples | 48 h | 72 h | 48 h | 72 h |
| Icaritin | 20.59 ± 2.82 | 16.05 ± 1.27 | 23.24 ± 1.02 | 14.96 ± 0.66 |
| 35 | >100 | >100 | >100 | >100 |
| 39 | >100 | 5.56 ± 0.60 | >100 | 6.65 ± 1.29 |
| 44 | >100 | 10.11 ± 0.32 | >100 | >100 |
| 50 | 24.11 ± 3.06 | 25.32 ± 2.11 | >100 | 46.49 ± 3.43 |
| 51 | 14.86 ± 0.22 | 7.09 ± 0.38 | 9.30 ± 0.35 | 5.99 ± 0.49 |
| 57 | 10.31 ± 1.19 | 7.91 ± 0.98 | 10.31 ± 1.19 | 5.823 ± 1.04 |
| 58 | 19.70 ± 0.52 | 19.98 ± 2.12 | >100 | 2.51 ± 0.72 |
| 64 | >100 | 32.57 ± 3.12 | >100 | >100 |
| 65 | >100 | 37.74 ± 0.20 | >100 | >100 |
| 71 | >100 | >100 | >100 | 29.69 ± 1.23 |
| 72 | >100 | >100 | >100 | >100 |
| 73 | 43.24 ± 1.21 | 36.77 ± 0.65 | >100 | 37.24 ± 1.36 |
| 81 | >100 | 11.99 ± 1.23 | >100 | 22.220 ± 1.45 |
| 87 | >100 | 7.03 ± 0.67 | >100 | 12.68 ± 1.02 |
| 88 | >100 | >100 | >100 | 40.470 ± 1.56 |
| 89 | >100 | >100 | >100 | 35.644 ± 1.36 |
| 95 | >100 | >100 | >100 | 5.644 ± 0.65 |
| 96 | 11.99 ± 0.82 | 5.56 ± 0.80 | 29.69 ± 1.23 | 6.65 ± 1.29 |
| 97 | >100 | >100 | >100 | 8.68 ± 0.52 |
| 103 | >100 | 5.45 ± 0.53 | >100 | 32.93 ± 1.12 |
| 104 | >100 | 9.02 ± 0.27 | >100 | 12.9 ± 1.03 |
| 105 | 15.05 ± 1.32 | 7.69 ± 0.04 | >100 | 9.57 ± 1.20 |

It can be known from the calculation results (Table 2) that, the IC 50 of Icaritin on human hepatoma cells HepG2 and SMMC7721 at 48 h were 20.59±2.82, 23.24±1.02 μM, respectively; the IC₅₀ at 72 h were 16.05±1.27, 14.96±0.66 μM, respectively; the IC₅₀ values of compounds 51, 57, 96, 105 at 48, 72 h were all less than those of Icaritin, indicating that the anti-hepatoma activities of compounds 51, 57, 96, 105 were higher than that of the bulk drug. For human hepatoma cells HepG2, at 72 h after administration, the IC₅₀ values of compounds 39, 44, 51, 57, 81 87, 96, 103, 104, 105 were all less than that of Icaritin; for human hepatoma cells SMMC7721, at 72 h after administration, the IC₅₀ values of compounds 39, 51, 57, 58, 87, 95, 96, 97, 104, 105 were all less than that of Icaritin; the results indicate that structural modification of Icaritin could effectively improve its activity.

### EXAMPLE 25

### Anti-tumor Activity in Vivo

According to the investigation results of solubility and cell activity, compounds 51 and 57 with high solubility and strong cell activity were selected to evaluate the anti-hepatoma activity in vivo on HepG2 nude mouse models.

Human hepatoma cells HepG2 were cultivated to logarithmic growth phase in DMEM culture medium containing 10% fetal calf serum and 1% antibiotics (penicillin and streptomycin), and digested by adding 0.25% pancreatin. After the resulting cells were cultivated in DMEM and the pancreatin was removed, they were centrifuged at 1000 rpm for 3 min. Then the cells were collected and washed with free medium until no bubbles produced as observed by naked eyes, and then ° entrifuged. After discarding the supernatant, the cells were resuspended with free medium adjust the cell density to 1×10⁶/100 μL.

70 Balb/c nude mice raised in an SPF-grade animal house were taken. 100 μL, HepG2 cells were inoculated subcutaneously at the right lateral rib of each nude mouse. The growing status of nude mice was observed and the subcutaneous tumor volume was measured every day. One week after inoculation, obvious nodules were formed subcutaneously in nude mice. Administration was initiated when the tumor grew to 100 mm³.

Tumor volume increased to 80-120 mm³ at two weeks after inoculation, marked as Day 0. The nude mice were randomly divided into 6 groups with 6 mice in each group: a normal saline group (control), an Icaritin group (Icarian), a yyhs-i-b high dose group, a yyhs-1-b low dose group, a yyhs-2-a high dose group, and a yyhs-2-a low dose group. Administration was performed intraperitoneally every day for 21 days totally.

The administration doses were: Icarian at 10 mg/kg, compound 51 high dose at 25.30 mg/kg, low dose at 12.65 mg/kg, compound 57 high dose at 22.91 mg/kg, and low dose at 11.45 mg/kg.

From day 0, the body weights and tumor volumes of nude mice were measured once every three days, in which the maximum diameter (a) of tumor was measured first, and then the longest radial line length (b) perpendicular to the maximum diameter line was measured, in mm, thereby calculating the tumor volume according to the following formula:

$$V ( {mm}^{3} ) = \frac{a \times {b}^{2}}{2}$$

At the end of the treatment cycle, blood was taken from orbital vein of the nude mice for later blood routine test. Then the nude mice were sacrificed using cervical vertebra dislocation. After taking photos, the tumors were stripped immediately and photographed for record. From day 0, the body weights of each group of nude mice were weighed and recorded every 3 days, and at the same time, the tumor volumes of these nude mice were recorded. The average body weight and tumor volume were calculated for each group, and the curve of body weight versus tumor volume of the nude mice was plotted according to the statistical data.

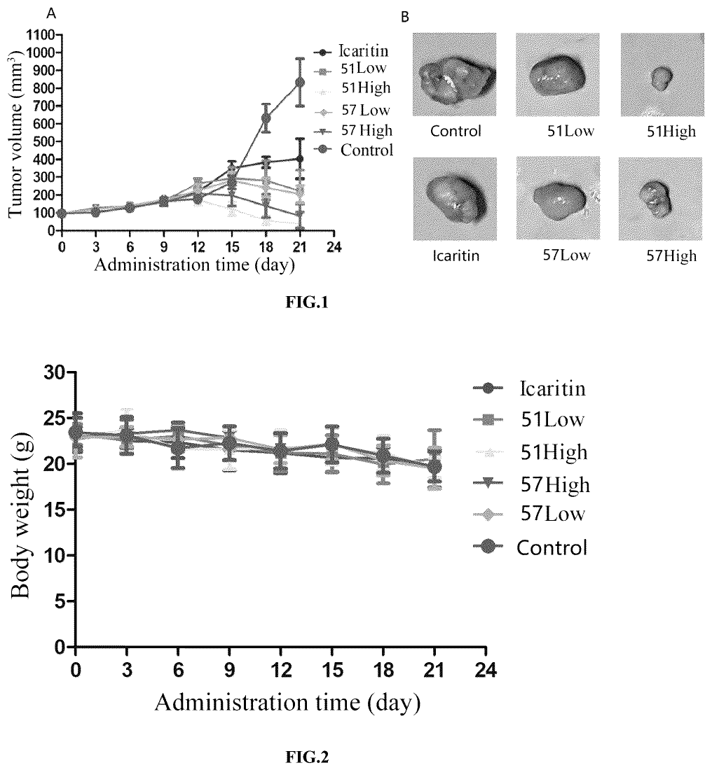

The hearts, livers, spleens, lungs, and kidneys were subsequently picked from the nude mice, soaked with 4% paraformaldehyde and then preserved at a low temperature of 4° C. for later investigation of the mechanism of the anti-tumor in vivo. The tumor volume growth curve was plotted with the time as the horizontal coordinate, and with the tumor volume as the vertical coordinate. The tumor volume growth curves and the appearance of tumors are seen in FIG. 1. The body weight changing curves of mice are plotted in FIG. 2, with the time as the horizontal coordinate, and the body weight of mice as the vertical coordinate.

It can be known from FIG. 1 that, compounds 51 and 57 have excellent anti-tumor activities in vivo and are dose-dependent, and have significantly better therapeutic effects than that of the bulk drug Icaritin. Compared with an equal mole of Icaritin, the tumor inhibition rates of compounds 51 and were 95% and 90% respectively, but the tumor inhibition rate of Icaritin was 53%, indicating that the anti-tumor activity of Icaritin could be significantly improved, through structural modification.

It can be known from FIG. 2 that, during the administration, there was no significant difference among the body weight changes of each group of mice, indicating that the compounds have relatively high safety.

What is claimed is:

1. A flavone derivative represented by formula I, 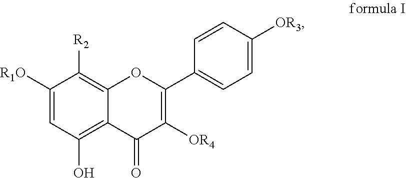
in which,
R¹ is selected from the group consisting of H, C₁₋₄ alkyl, amino, and C₁₋₄ acyl;
R₂ is isopentenyl or 2-hydroxy isopentyl;
R₃ is selected from the group consisting of H, methyl, and deuterated methyl; and
R₄ is selected from the group consisting of C₁₋₄ alkyl, amino, C₁₋₄ acyl, and 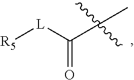
wherein R₅ represents a monosaccharide residue or an oligosaccharide residue;
L is selected from the group consisting of a polypeptide, C₁-C₂₀ linear alkyl or a derivative thereof, a derivative of C₁-C₂₀ linear or branched acyl, C₁-C₂₀ glycol or a derivative thereof, 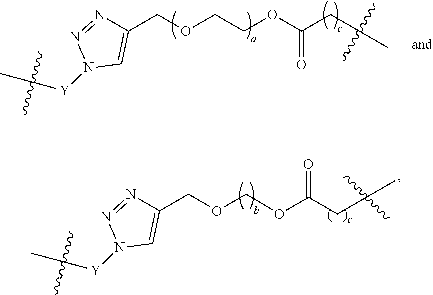
wherein Y is 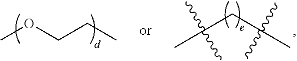
a is an integer from 0-100, b is an integer from 1-100, c is an integer from 1-10, d is an integer from 0-100, and e is an integer from 0-100,
and a pharmaceutically acceptable salt, hydrate or solvate thereof.
The derivative of the present disclosure has an efficient and broad-spectrum anti-cancer activity.

2. The flavone derivative of claim 1, wherein:
R₁ represents H;
R₂ represents isopentenyl;
R₃ represents methyl; and
R₄ represents 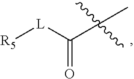
wherein R₅ is selected from the group consisting of the monosaccharide residue and the oligosaccharide residue, preferably the following monosaccharide residues 1-24 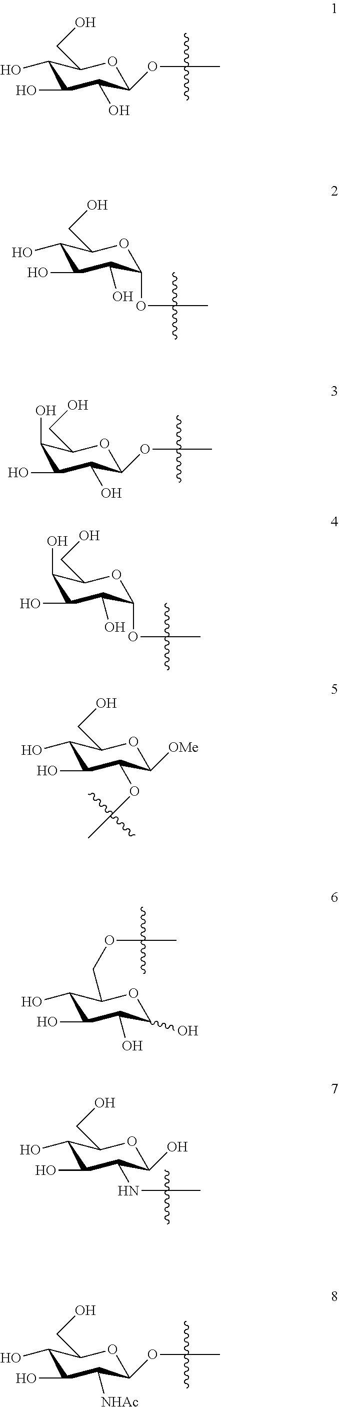 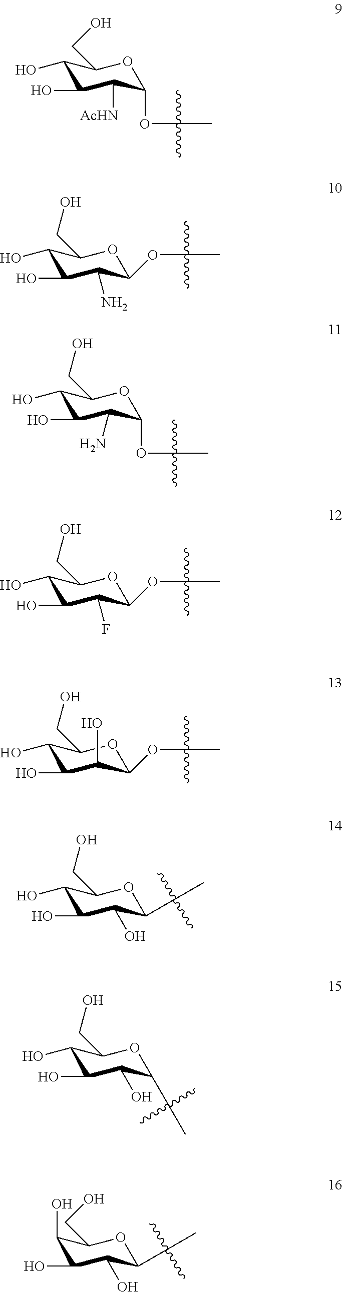 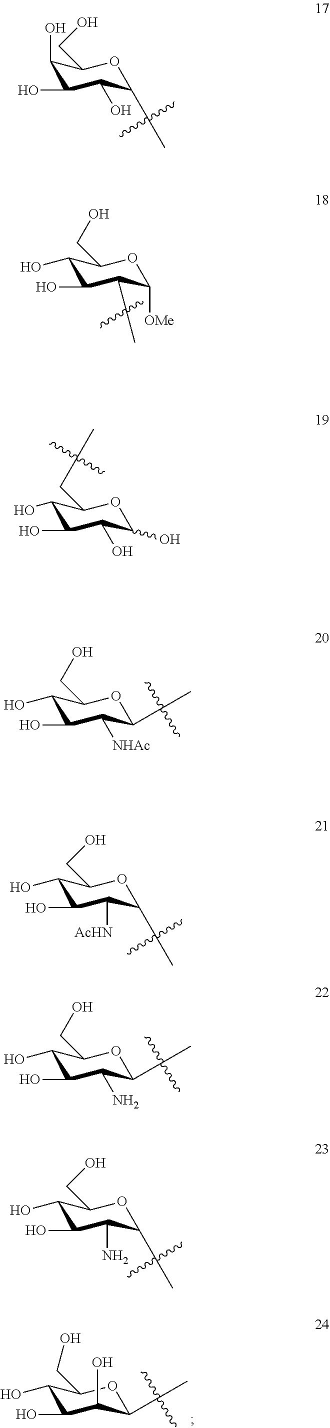
L is selected from the group consisting of the polypeptide, C₁-C₂₀ linear alkyl or the derivative thereof ; the derivative of C₁-C₂₀ linear or branched acyl, C₁-C₂₀ glycol or the derivative thereof, 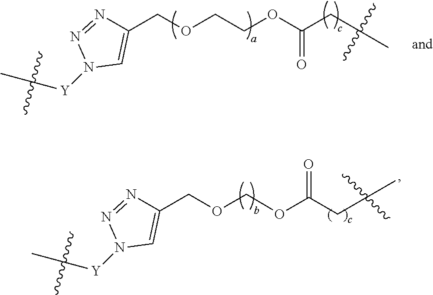
wherein Y is 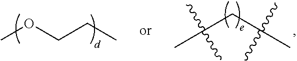
a is an integer from 0-20, b is an integer from 1-20, c is an integer from 1-10, d is an integer from 0-20, and e is an integer from 0-20.

3. The flavone derivative of claim 1 and 2, wherein:
R₁ represents H;
R₂ represents isopentenyl;
R₃ represents methyl; and
R4 represents 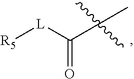
wherein R₅ is selected from the group consisting of monosaccharide residues 1. 2, 14 and 15; L is selected from the group consisting of 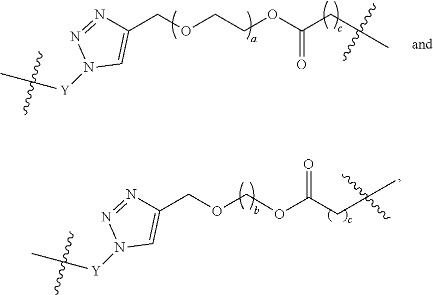
wherein Y is 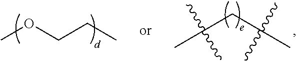
a is an integer from 0-20, b is an integer from 1-20, c is an integer from 2-6, d is an integer from 0-20, and e is an integer from 0-20.

4. The flavone derivative of any one of claims 1-3, wherein the flavone derivative is one of the following compounds:

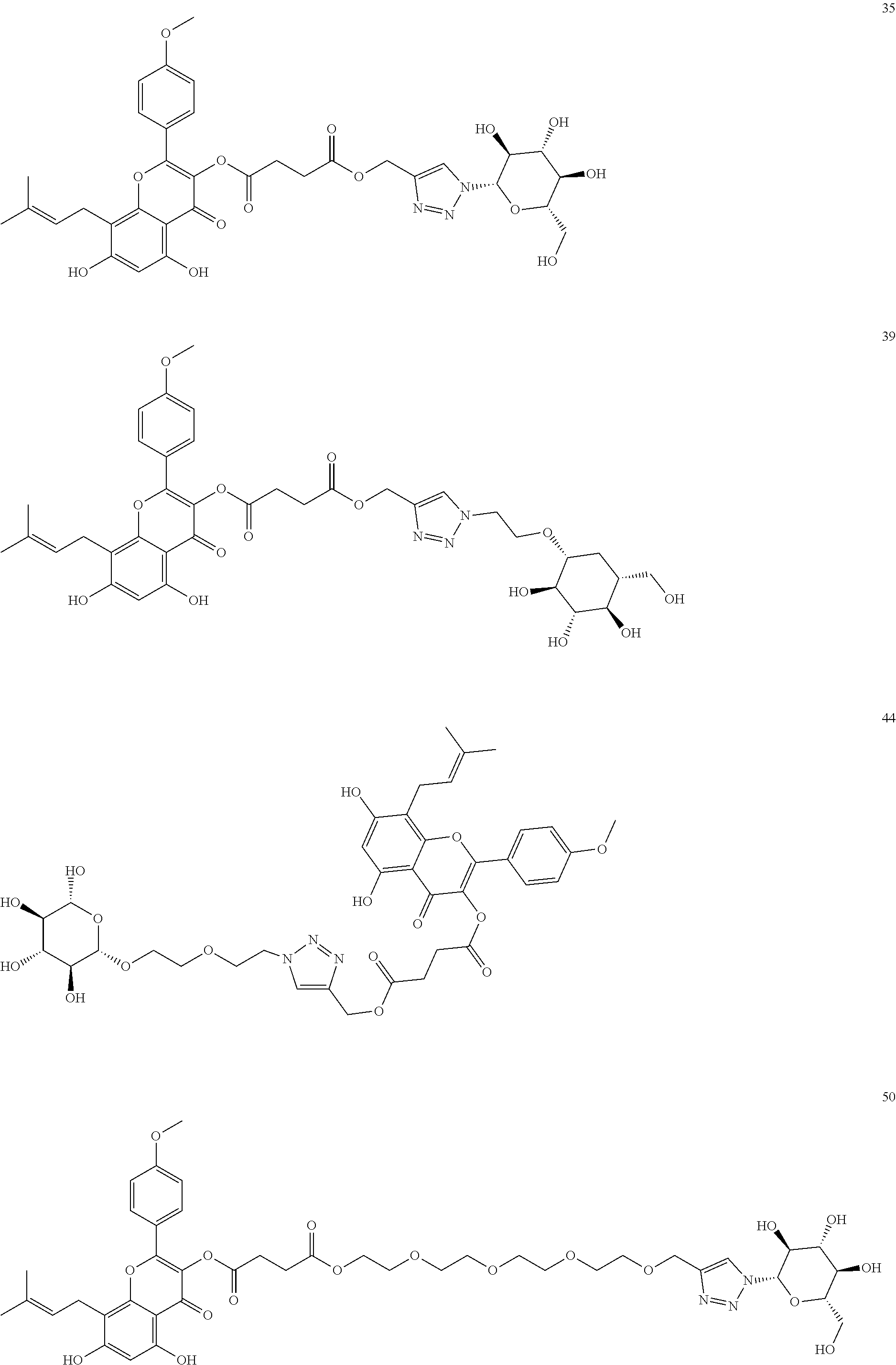

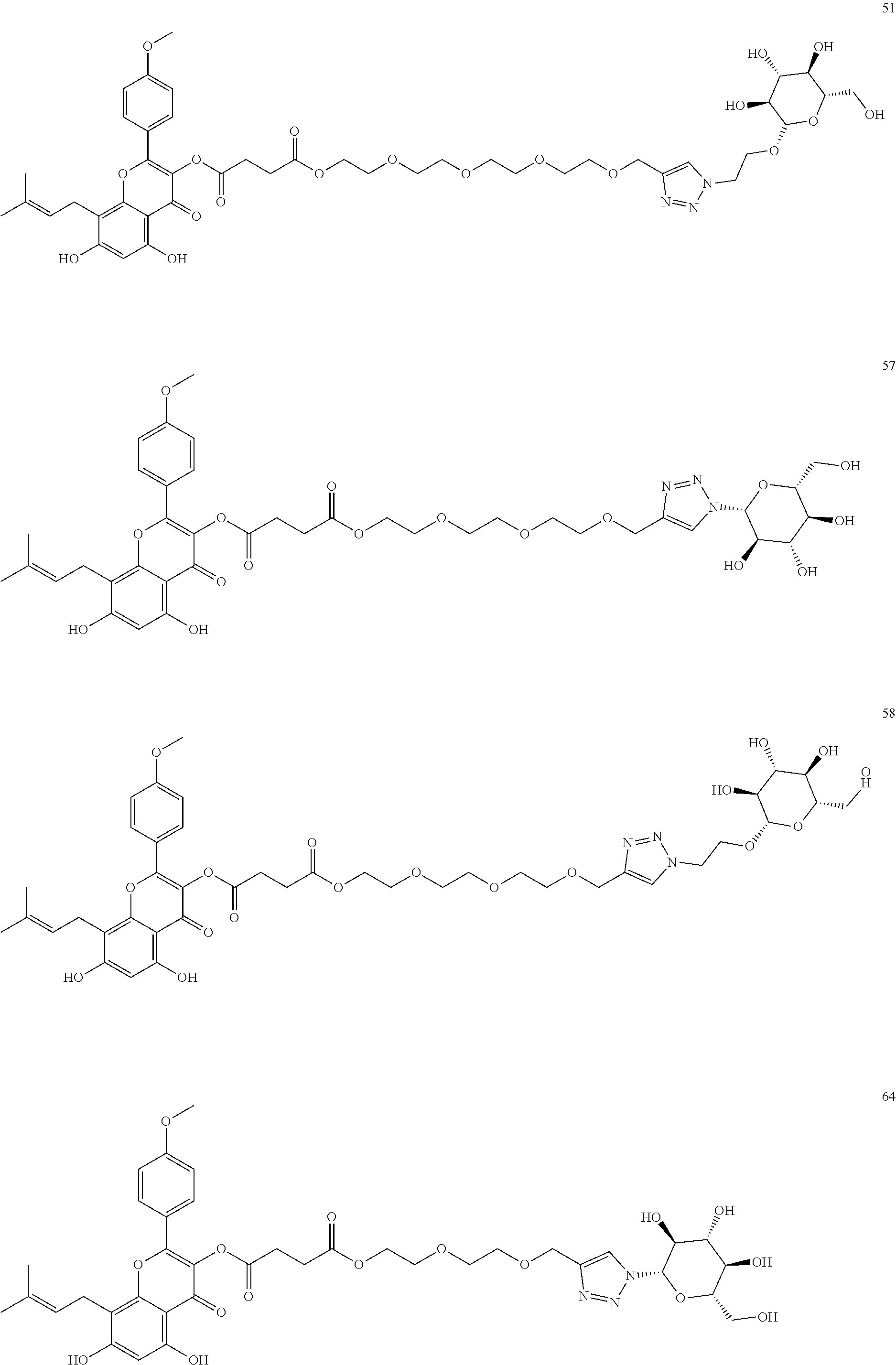

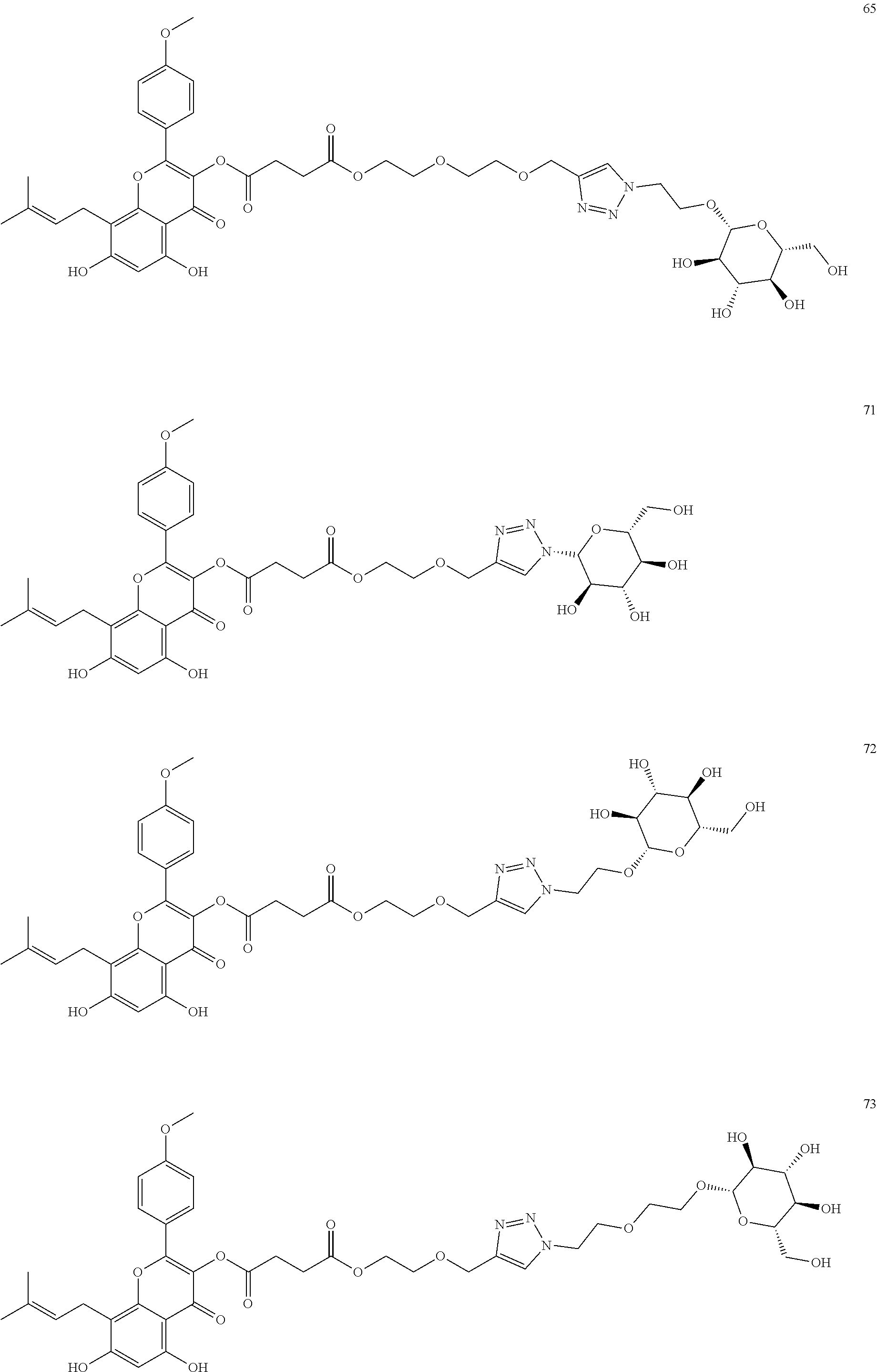

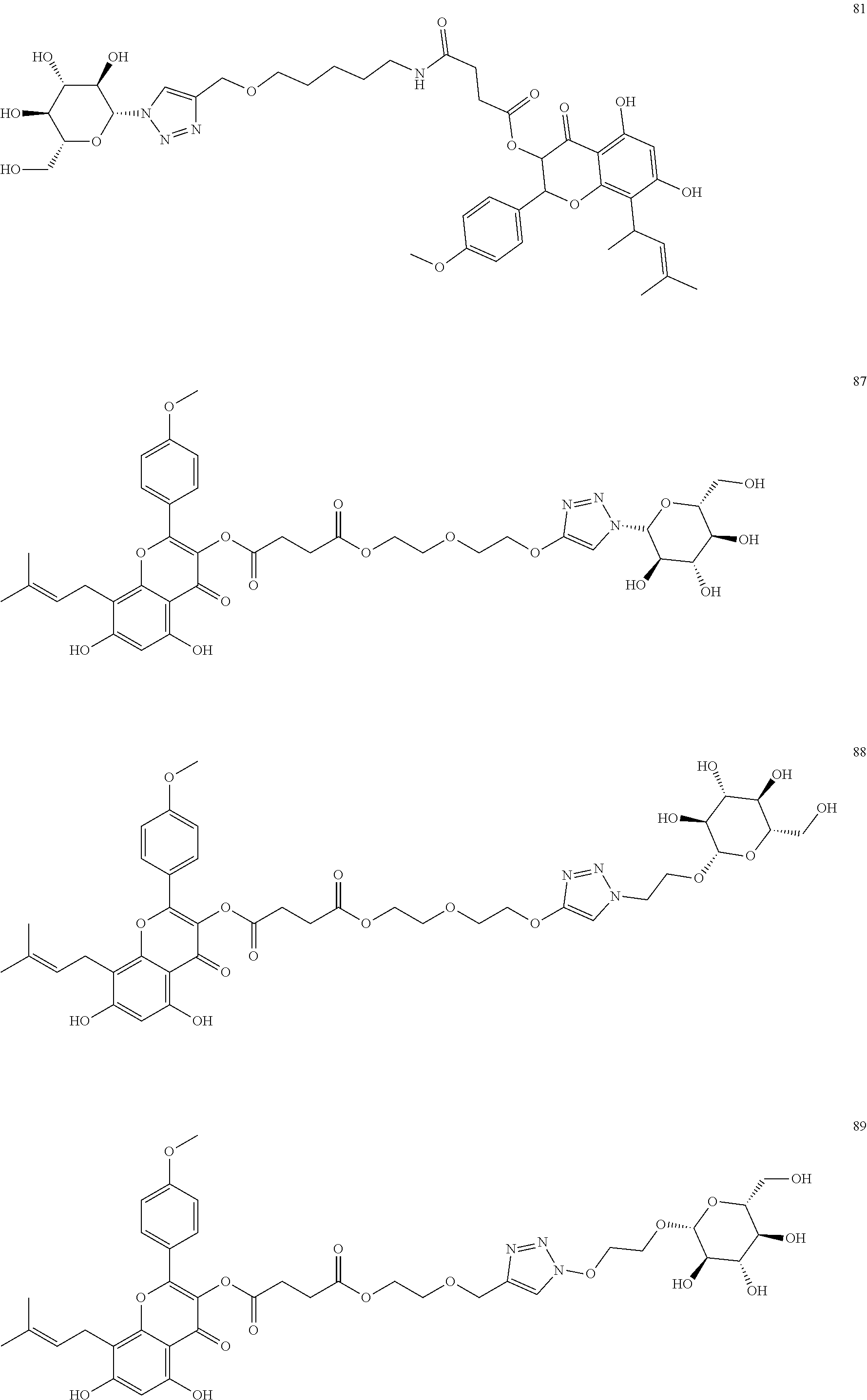

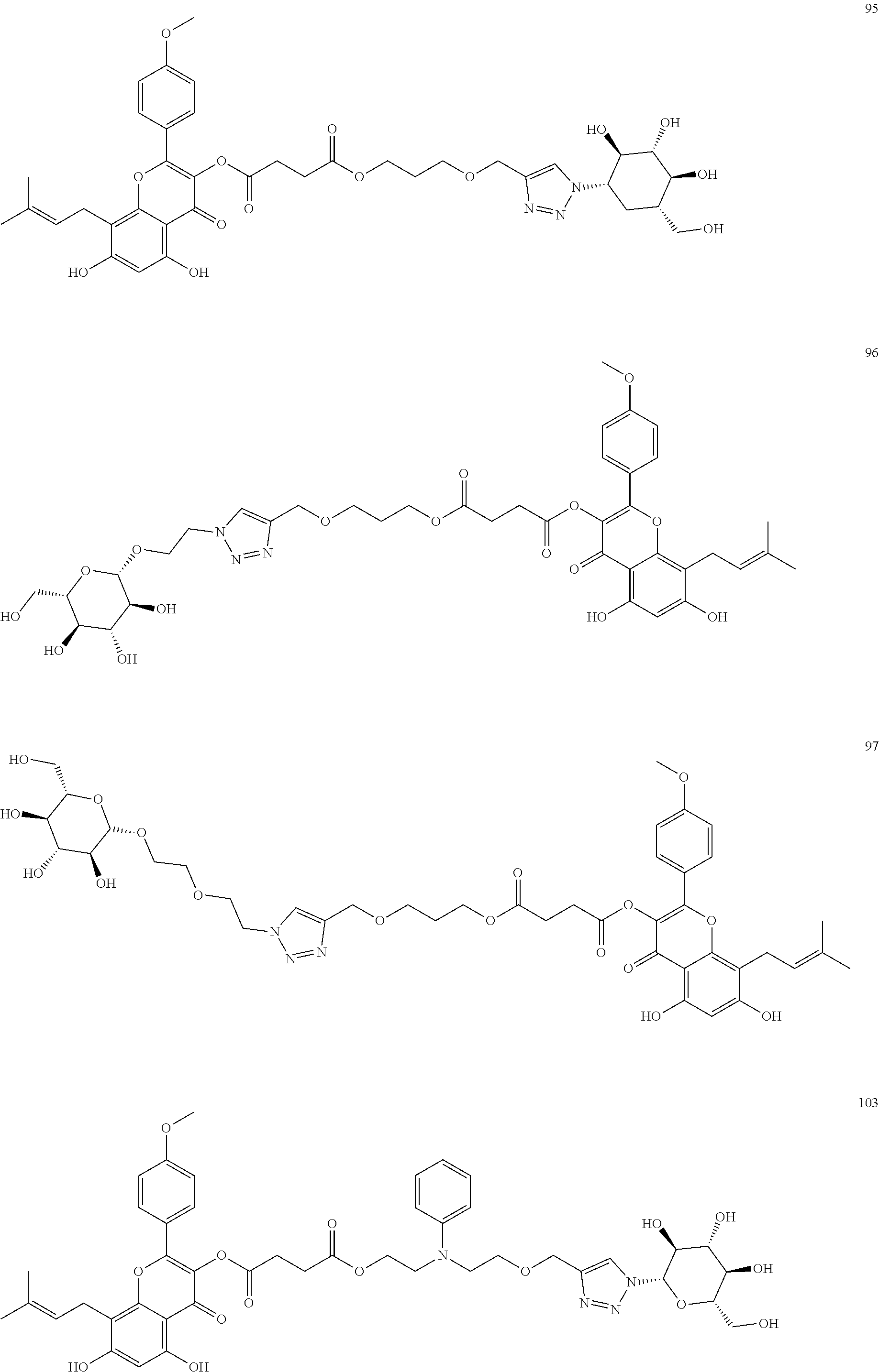

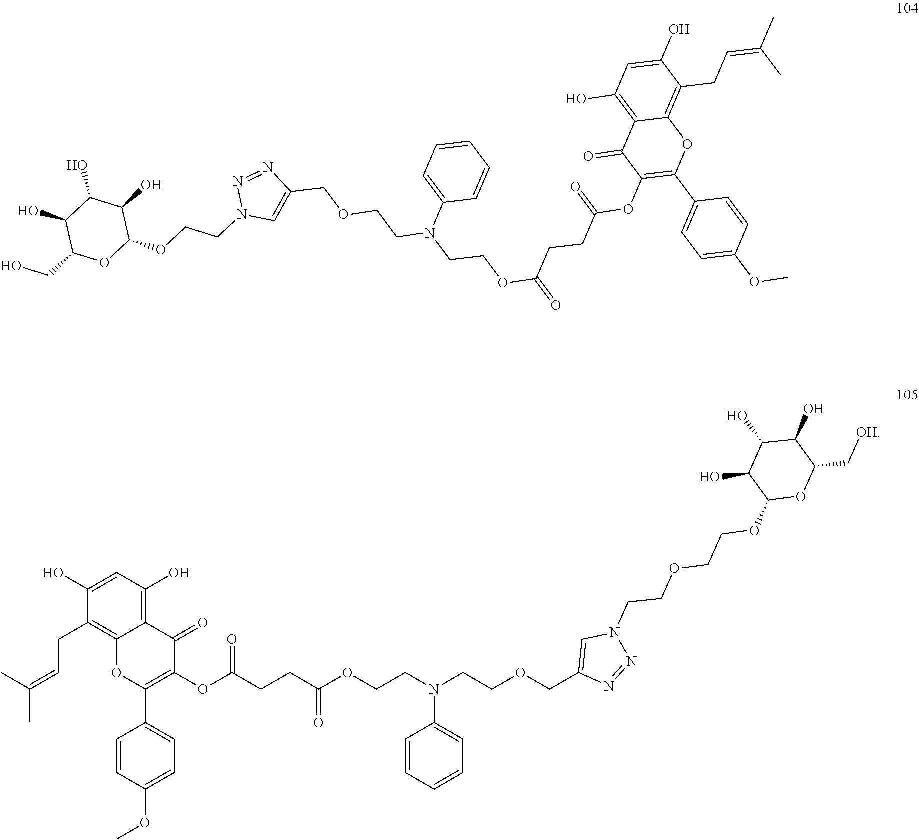

5. A tumor cell growth inhibitor, which is prepared from the flavone derivative of any one of claims 1-4.

6. The tumor cell growth inhibitor of claim 5, wherein the tumor comprises one of liver cancer, colorectal tumor, lung tumor and breast tumor, gastric cancer, esophagus cancer, leukemia, prostatic cancer, osteosarcoma, cervical cancer, thyroid cancer, ovarian cancer, and pancreatic cancer.

7. The use of claim 6, wherein the cancer is liver cancer.

8. The use of claim 7, wherein the anti-cancer drug is in any pharmaceutically acceptable dosage form, and the tumor cell growth inhibitor further comprises pharmaceutically acceptable carriers and/or auxiliaries.
